(19) United States
(12) Patent Application Publication
(10) Pub. No.: US 2019/0152962 A1
(43) Pub. Date: May 23, 2019

(54) CYCLOPROPYL DERIVATIVES AS ROR-GAMMA MODULATORS

(71) Applicant: CADILA HEALTHCARE LIMITED, Ahmedabad, Gujarat (IN)

(72) Inventors: Ranjit DESAI, Ahmedabad, Gujarat (IN); Sanjay S. KUMAR, Ahmedabad, Gujarat (IN); Vrajesh PANDYA, Ahmedabad, Gujarat (IN); Jigar DESAI, Ahmedabad, Gujarat (IN); Saurin RAVAL, Ahmedabad, Gujarat (IN)

(21) Appl. No.: 16/316,220

(22) PCT Filed: Jul. 13, 2017
(86) PCT No.: PCT/IB2017/054239

§ 371 Date: Jan. 8, 2019

(30) Foreign Application Priority Data
Jul. 14, 2016 (IN) 201621024153
Jul. 14, 2016 (IN) 201621024154

(51) Int. Cl. C07D 413/12; C07D 271/10; C07D 413/14; C07D 413/04; C07D 271/06; C07D 263/56; C07D 263/32; A61P 37/06

(52) U.S. Cl. CPC C07D 413/12; C07D 271/10; C07D 413/14; C07D 413/04; A61K 45/06; C07D 263/56; C07D 263/32; A61P 37/06; C07D 271/06

(57) ABSTRACT

The present invention provides compounds which are modulators of RORγ and their use for the treatment of diseases or conditions mediated by RORγ. Further, the present invention relates to processes of preparing such compounds, their tautomeric forms, novel intermediates involved in their synthesis, their pharmaceutically acceptable salts, methods for using such compounds and pharmaceutical compositions containing them (Formula I).

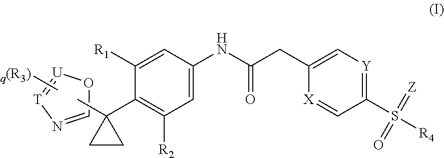

### FIELD OF INVENTION

The present invention relates to novel derivatives of the general formula (I) as modulators of RORγ, their tautomeric forms, their stereoisomers, their pharmaceutically acceptable salts, pharmaceutical compositions containing them, methods for their preparation, use of these compounds in medicine and the intermediates involved in their preparation.

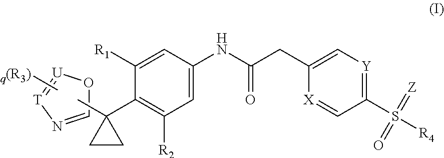

### BACKGROUND OF THE INVENTION

The Retinoic acid receptor-related orphan receptor γ known as RORγ belonging to the nuclear receptor superfamily (Hirose, T.; Smith, R. J.; Biochem. Biophys. Res. Commun. 1994, 205, 1976-1983). There are three sub-types of ROR's classified as RORα, RORβ and RORγ. As observed in majority of other nuclear receptors, structure of ROR's consists of four distinct regions called N-terminal A/B domain, a DNA binding domain, a hinge domain and a ligand binding domain. Two isoforms RORγ1 and RORγ2 (which is also called as RORγt) have been identified that differ only in N-terminal sequences (He, Y.-W.; Deftos, M. L.; Ojala, Immunity 1998, 9,797-806). Tissue distribution of these two isoforms are quite distinct, while RORγ1 is expressed in a variety of tissues including thymus, liver, kidney and muscle, RORγt is exclusively expressed in the cells of the immune system. The isoform RORγt plays important role in the development and regulation of the immune system through its regulatory effect on T helper cells (Th17 cell) (Ivanov, I. I.; McKenzie, B. S.; Zhou, L.; Cell 2006, 126, 1121-1133).

Th17 is the IL-17 producing CD4+ Th subset and are key drivers of chronic inflammation in autoimmune diseases such as multiple sclerosis, rheumatoid arthritis, irritable bowel diseases, psoriasis, psoriatic arthritis (Jetten (2009) Nucl. RecepL Signal. 7: e003; Manel et al. (2008) Nat. Immunol. 9: 641-649). Mouse autoimmune disease models like experimental autoimmune encephalomyelitis (EAE) and collagen-induced arthritis (CIA) have demonstrated the role of Th17 in autoimmune diseases. RORγ is central transcription factor driving Th17 differentiation.

The significant role of RORγ in the pathogenesis of autoimmune diseases forms a basis for the development of ligands which can modulate RORγ activity and could lead to specific therapies for diseases mediated by RORγ.

WO2012100732 discloses thiophene derivatives represented by following formula as RORγ modulators.

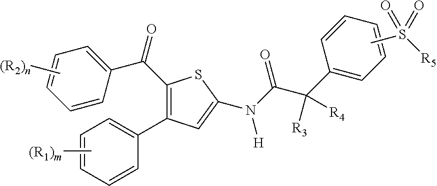

WO2012100734 and WO201227965 disclose compounds of the following formula as RORγ modulators.

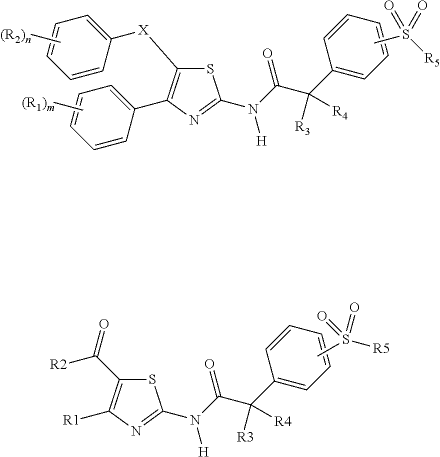

WO2013029338 discloses biaryl modulators of RORγ having following formula and their use in treatment of disease mediated by RORγ.

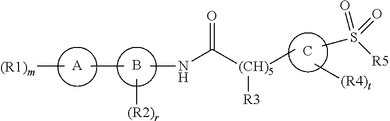

WO2013171729 discloses aryl or heteroaryl carboxamides with following formula and their use as RORγ modulators.

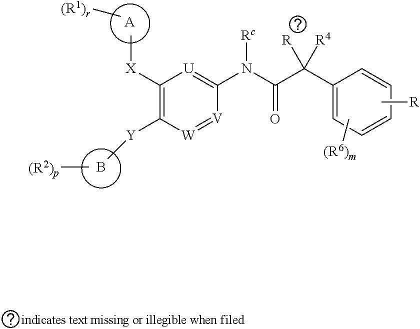

WO2014125426 discloses trisubstituted heterocyclic derivatives having following formula as RORγ modulators.

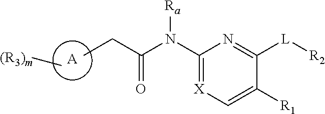

WO2014179564 discloses thiazolopyrrolidine derivatives with following formula for the treatment of diseases mediated by RORγ.

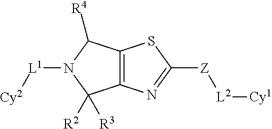

WO2015083130 and WO2015101928 disclose fused pyridine/pyrimidine derivatives and fused thiophene/thiazole derivatives respectively with following formula as RORγ modulators.

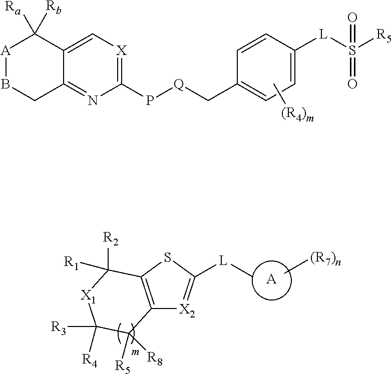

WO2015159233 discloses aryl and heteroaryl ether compounds with following formula as RORγ modulators.

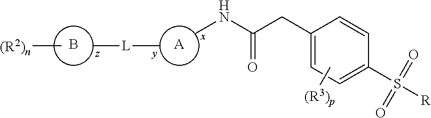

WO2015145371 discloses following types of RORγ modulators and their uses in the treatment of disease mediated by RORγ.

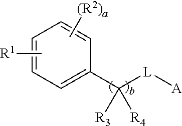

WO2015116904 discloses dihydropyrrolopyridine inhibitors of RORγ with following formula.

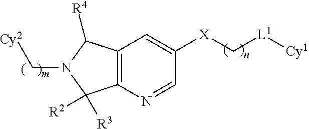

WO2016193470, WO2016193468, WO2016193461, WO2016193459 and WO2016193452 disclose substituted acetamide derivatives as RORγ modulators.

WO2017024018 and WO2017087608 disclose modulators of RORγ with following general formulae.

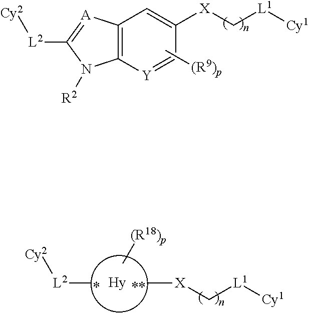

WO2017010399 discloses compounds with following formula having RORγt inhibitory effects.

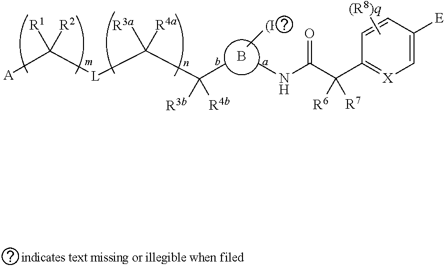

Though several compounds have been reported in the literature as RORγ modulators, none of these compounds have reached the market. Looking at the significant unmet medical need for such compounds based on their potential beneficial effects as discussed above, identification of further compounds which can act as RORγ modulators and which will have one or more superior benefits over the earlier known compounds is warranted.

Our endeavor to identify potent and efficacious RORγ modulators started with the synthesis of molecules A and B shown in Table 1. Following synthesis, they have been evaluated in luciferase assay to evaluate RORγt inhibitory activity. Additionally, their ability to inhibit production of IL-17, a key culprit for autoimmune diseases has also been studied. The data is provided below:

TABLE 1

| Structure | IC₅₀ (Luciferase assay) | IC₅₀ (IL-17 assay) |
|---|---|---|
| | 82.3 nM | >5 μM |
| | 299 nM | 2.1 μM |

The compound A having hydrogen atoms on central phenyl ring at ortho position to cyclopropyl ring has been found to inhibit RORγt with an IC₅₀ of 82.3 nM. When we replaced one of the hydrogen with chlorine atom as shown in B, IC₅₀ value further dropped to 299 nM. However, both the compounds were found to be poor in IL-17 inhibition assay as reflected in their IC₅₀ values (IC₅₀ for A: >5 μM, IC₅₀ for B: 2.1 μM).

To our surprise, when we substituted both the ortho positions of phenyl ring with groups such as halogen or methyl, as represented in the compounds of the present invention (I), dramatic improvement in activity was observed in both the biological assays. Activity data of these compounds are provided below in Table 4.

### SUMMARY OF THE INVENTION

The present invention discloses novel compounds as defined by the general formula (I) that modulates the activity of RORγ and provides treatment option for autoimmune and/or inflammatory diseases such as multiple sclerosis, rheumatoid arthritis, irritable bowel diseases, psoriasis, psoriatic arthritis and the like which are mediated by RORγ. The compounds of the present invention are useful in the treatment of the human or animal body, by regulation of RORγ receptor gene expression. The compounds of this invention are therefore suitable for the treatment/mitigation/regulation or prophylaxis of number of autoimmune or inflammatory diseases mentioned above.

### Embodiments of the Invention

The main objective of the present invention is to provide novel compounds of general formula (I), their tautomeric forms, novel intermediates involved in their synthesis, their pharmaceutically acceptable salts, their pharmaceutically acceptable solvates and pharmaceutical compositions containing them or their suitable mixtures, suitable for the treatment of autoimmune and/or inflammatory diseases such as multiple sclerosis, rheumatoid arthritis, irritable bowel diseases, psoriasis, psoriatic arthritis and the like.

In an embodiment is provided a process for the preparation of novel compounds of general formula (I), their tautomeric forms, novel intermediates involved in their synthesis, their pharmaceutically acceptable salts, pharmaceutically acceptable solvates and pharmaceutical compositions containing them.

In another embodiment is provided pharmaceutical compositions containing compounds of general formula (I), their tautomeric forms, their pharmaceutically acceptable salts, solvates and their mixtures along with suitable pharmaceutically acceptable carriers, solvents, diluents, excipients and other media normally employed in their manufacture and formulations.

In a still further embodiment is provided the use of the novel compounds of the present invention for the treatment of autoimmune diseases and/or inflammatory diseases, by administering a therapeutically effective & non-toxic amount of the compound of formula (I), or their pharmaceutically acceptable compositions to the mammals.

In a further embodiment is provided a method of treatment of diseases which can be treated or whose symptoms can be reversed with by administering a therapeutically effective & non-toxic amount of the compound of formula (I) or their pharmaceutically acceptable compositions to the mammals.

### DETAILED DESCRIPTION OF THE INVENTION

In a first embodiment, the present invention relates to compounds of the general formula (I),

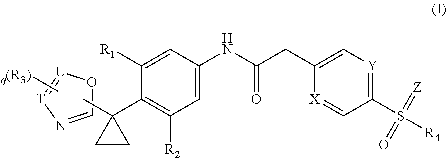

Wherein

R₁ and R₂ are each independently selected from halogen and (C₁-C₃)alkyl;

R₃ at each occurrence is independently selected from hydrogen, (C₁-C₆)alkyl, halo(C₁-C₆)alkyl, (C₆-C₁₀)aryl, (C₆-C₁₀)heteroaryl, (C₃-C₆)cycloalkyl, (C₄-C₆) heterocyclyl;

R₄ is selected from (C₁-C₃)alkyl, —NHR₆;

X and Y are each independently selected from CH or N atom;

Z is selected from NH or O atom;

Each of T and U is independently selected from C or N atom with the proviso that both T and U cannot be simultaneously N atom.

In an alternate embodiment when R₃ is phenyl and both T and U represent carbon, then T and U can be fused with a phenyl ring to form radical of the following formula;

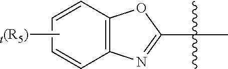

R₅ at each occurrence is independently selected from the group comprising of hydrogen, hydroxy, cyano, halogen, halo(C₁-C₆)alkyl, optionally substituted (C₁-C₆)alkyl, —O(C₁-C₆alkyl), (C₃-C₆)cycloalkyl;

R₆ is (C₃-C₆)cycloalkyl;

In an embodiment, when R₃ is substituted, the substituents on R₃ is selected from the group comprising of hydrogen, hydroxy, cyano, halogen, —OCF₃, halo(C₁-C₆)alkyl, optionally substituted (C₁-C₆)alkyl, —O(C₁-C₆alkyl), (C₃-C₆)cycloalkyl;

In an embodiment, the (C₁-C₆) alkyl chain as used herein before, may further be substituted with hydrogen, hydroxy, —COOH, cyano, halo, oxo, imino, haloalkyl, (C₂-C₆)alkenyl, (C₂-C₆)alkynyl, (C₃-C₆)cycloalkyl, aryl, heterocyclyl, heteroaryl;

q represents integers from 1-2;

t represents integers from 1-4;

Further preferred embodiments are those disclosed below.

Preferred R₁ and R₂ is Cl and CH₃;

Preferred R₃ is selected from (C₆-C₁₀)aryl, (C₃-C₆)cycloalkyl and (C₄-C₆) heterocyclyl;

In a second embodiment, the compounds of formula (I) is of formula (I-A);

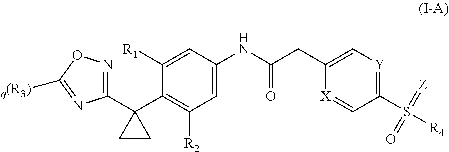

or pharmaceutically acceptable salt thereof, wherein the groups R₁ to R₄, X, Y and Z in formula (I-A) are as described for formula (I).

In a third embodiment, the compounds of formula (I) is of formula (I-B);

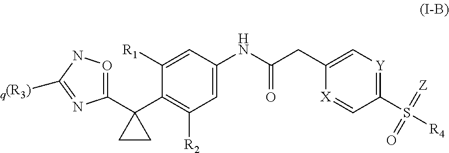

or pharmaceutically acceptable salt thereof, wherein the groups R₁ to R₄, X, Y and Z in formula (I-B) are as described for formula (I).

In a fourth embodiment, the compounds of formula (I) is of formula (I-C);

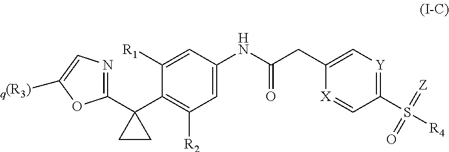

or pharmaceutically acceptable salt thereof, wherein the groups R₁ to R₄, X, Y and Z in formula (I-C) are as described for formula (I).

In a fifth embodiment, the compounds of formula (I) is of formula (I-D);

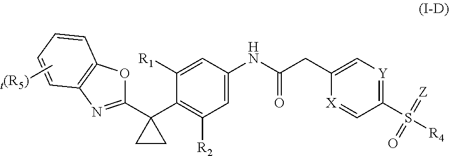

or pharmaceutically acceptable salt thereof, wherein the groups R₁ to R₄, X, Y and Z in formula (I-D) are as described for formula (I).

In a further embodiment the groups, radicals described above may be selected from:
the “alkyl” group used either alone or in combination with other radicals, denotes a linear or branched radical containing one to eight carbons, selected from methyl, ethyl, n-propyl, iso-propyl, n-butyl, sec-butyl, tert-butyl, amyl, t-amyl, n-pentyl, n-hexyl, and the like;
the “alkenyl” group used either alone or in combination with other radicals, is selected from a radical containing from two to eight carbons, more preferably groups selected from vinyl, allyl, 2-butenyl, 3-butenyl, 2-pentenyl, 3-pentenyl, 4-pentenyl, 2-hexenyl, 3-hexenyl, 4-hexenyl and the like; the “alkenyl” group includes dienes and trienes of straight and branched chains;
the “alkynyl” group used either alone or in combination with other radicals, is selected from a linear or branched radical containing two to eight carbon atoms, more preferably thienyl, 1-propynyl, 2-propynyl, 1-butynyl, 2-butynyl, 3-butynyl, 1-pentynyl, 2-pentynyl, 3-pentynyl, 4-pentynyl, 1-hexynyl, and the like. The term “alkynyl” includes di- and tri-ynes;
the “cycloalkyl”, or “alicyclic” group used either alone or in combination with other radicals, is selected from a cyclic radical containing three to six carbons, more preferably cyclopropyl, cyclobutyl, cyclopentyl, cyclohexyl and the like;
the “haloalkyl” group is selected from an alkyl radical, as defined above, suitably substituted with one or more halogens; such as fluoromethyl, difluoromethyl, trifluoromethyl, fluoroethyl, difluoroethyl, trifluoroethyl, mono or polyhalo substituted methyl, ethyl, propyl, butyl, pentyl or hexyl groups;
the “aryl” or “aromatic” group used either alone or in combination with other radicals, is selected from a suitable aromatic system containing one, two or three rings wherein such rings may be attached together in a pendant manner or may be fused, more preferably the groups are selected from phenyl, naphthyl, tetrahydronaphthyl, indane, biphenyl, and the like;
the “heterocyclyl” or “heterocyclic” group used either alone or in combination with other radicals, is selected from suitable saturated, partially saturated or unsaturated aromatic or non-aromatic mono, bi or tricyclic radicals, containing one or more heteroatoms selected from nitrogen, sulfur and oxygen, more preferably selected from aziridinyl, azetidinyl, pyrrolidinyl, imidazolidinyl, piperidinyl, piperazinyl, 2-oxopiperidinyl, 4-oxopiperidinyl, 2-oxopiperazinyl, 3-oxopiperazinyl, morpholinyl, thiomorpholinyl, 2-oxomorpholinyl, azepinyl, diazepinyl, oxapinyl, thiazepinyl, oxazolidinyl, thiazolidinyl, dihydrothiophene, dihydropyran, dihydrofuran, dihydrothiazole, benzopyranyl, benzopyranonyl, benzodihydrofuranyl, benzodihydrothienyl, pyrazolopyrimidonyl, azaquinazolinoyl, thienopyrimidonyl, quinazolonyl, pyrimidonyl, benzoxazinyl, benzoxazinonyl, benzothiazinyl, benzothiazinonyl, thieno piperidinyl, and the like; In one embodiment, the heterocycle group, wherever applicable, may consists of appropriate number of carbon atoms and include from 1-4 heteroatoms selected from the group consisting of N, O, and S(O)p, p=0-2, wherein the heterocycle may further be substituted with 1-2 carbonyl or 1-2 iminocarbonyl groups or one or more groups selected from R₇ wherein R₇ is selected from H, hydroxy, halogen, cyano, optionally substituted groups selected from (C₁-C₆)alkyl, alkoxy, amino, mono, di or trisubstituted amino, hydroxyalkyl, aminoalkyl, heterocyclylalkyl, aminocarbonyl groups;
the “heteroaryl” or “heteroaromatic” group used either alone or in combination with other radicals, is selected from suitable single or fused mono, bi or tricyclic aromatic heterocyclic radicals containing one or more hetero atoms selected from O, N or S, more preferably the groups are selected from pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzofuranyl, benzothienyl, indolinyl, indolyl, azaindolyl, azaindolinyl, pyrazolopyrimidinyl, azaquinazolinyl, pyridofuranyl, pyridothienyl, thienopyrimidyl, quinolinyl, pyrimidinyl, pyrazolyl, quinazolinyl, pyridazinyl, triazinyl, benzimidazolyl, benzotriazolyl, phthalazynil, naphthylidinyl, purinyl, carbazolyl, phenothiazinyl, phenoxazinyl, benzoxazolyl, benzothiazolyl and the like;
the “oxo” and “imino” group used either alone or in combination with other groups represents radical of formula —C═O or —C═NH respectively.

The compounds of formula (I) may optionally be converted to their suitable pharmaceutically acceptable salts by processes as are known in the art. The novel compounds of the present invention can further be formulated into suitable pharmaceutically acceptable compositions by combining with suitable excipients by techniques and processes and concentrations as are well known.

Suitable groups and substituents on the groups may be selected from those described anywhere in the specification.

Preferred compounds according to the present invention include but not limited to:
N-(3,5-dichloro-4-(1-(5-(4-fluorophenyl)-1,2,4-oxadiazol-3-yl)cyclopropyl)phenyl)-2-(4-(ethylsulfonyl)phenyl)acetamide;
N-(3,5-dichloro-4-(1-(5-phenyl-1,2,4-oxadiazol-3-yl)cyclopropyl) phenyl)-2-(4-(ethylsulfonyl)phenyl)acetamide;
N-(3,5-dichloro-4-(1-(5-(4-(trifluoromethyl)phenyl)-1,2,4-oxadiazol-3-yl)cyclopropyl) phenyl)-2-(4-(ethylsulfonyl)phenyl)acetamide;
N-(3,5-dichloro-4-(1-(5-(p-tolyl)-1,2,4-oxadiazol-3-yl)cyclopropyl) phenyl)-2-(4-(ethylsulfonyl)phenyl)acetamide;
N-(3,5-dichloro-4-(1-(5-(3,4-difluorophenyl)-1,2,4-oxadiazol-3-yl)cyclopropyl)phenyl)-2-(4-(ethylsulfonyl)phenyl)acetamide;
N-(3,5-dichloro-4-(1-(5-(3-fluorophenyl)-1,2,4-oxadiazol-3-yl)cyclopropyl)phenyl)-2-(4-(ethylsulfonyl)phenyl)acetamide;
N-(3,5-dichloro-4-(1-(5-(2,4-difluorophenyl)-1,2,4-oxadiazol-3-yl)cyclopropyl)phenyl)-2-(4-(ethylsulfonyl)phenyl)acetamide;
N-(3,5-dichloro-4-(1-(5-(2,4,6-trifluorophenyl)-1,2,4-oxadiazol-3-yl)cyclopropyl) phenyl)-2-(4-(ethylsulfonyl)phenyl)acetamide;
N-(3,5-dichloro-4-(1-(5-(2-chloro-4-fluorophenyl)-1,2,4-oxadiazol-3-yl)cyclopropyl) phenyl)-2-(4-(ethylsulfonyl)phenyl)acetamide;
N-(3,5-dichloro-4-(1-(5-(3-chloro-4-fluorophenyl)-1,2,4-oxadiazol-3-yl)cyclopropyl) phenyl)-2-(4-(ethylsulfonyl)phenyl)acetamide;
N-(3,5-dichloro-4-(1-(5-(3,4-dichlorophenyl)-1,2,4-oxadiazol-3-yl)cyclopropyl)phenyl)-2-(4-(ethylsulfonyl)phenyl)acetamide;
N-(3,5-dichloro-4-(1-(5-(2,4-dichiorophenyl)-1,2,4-oxadiazol-3-yl)cyclopropyl)phenyl)-2-(4-(ethylsulfonyl)phenyl)acetamide;
N-(3,5-dichloro-4-(1-(5-(4-chiorophenyl)-1,2,4-oxadiazol-3-yl)cyclopropyl)phenyl)-2-(4-(ethylsulfonyl)phenyl)acetamide;
N-(3,5-dichloro-4-(1-(5-(4-cyanophenyl)-1,2,4-oxadiazol-3-yl)cyclopropyl)phenyl)-2-(4-(ethylsulfonyl)phenyl)acetamide;
N-(3,5-dichloro-4-(1-(5-(2-fluoro-4-methoxyphenyl)-1,2,4-oxadiazol-3-yl) cyclopropyl) phenyl)-2-(4-(ethylsulfonyl)phenyl)acetamide;
N-(3,5-dichloro-4-(1-(5-(thiophen-2-yl)-1,2,4-oxadiazol-3-yl)cyclopropyl)phenyl)-2-(4-(ethylsulfonyl)phenyl)acetamide;
N-(3,5-dichloro-4-(1-(5-(5-chlorothiophen-2-yl)-1,2,4-oxadiazol-3-yl)cyclopropyl) phenyl)-2-(4-(ethylsulfonyl)phenyl)acetamide;
N-(3,5-dichloro-4-(1-(5-(5-methylthiophen-2-yl)-1,2,4-oxadiazol-3-yl)cyclopropyl) phenyl)-2-(4-(ethylsulfonyl)phenyl)acetamide;
N-(3,5-dichloro-4-(1-(5-(pyridin-3-yl)-1,2,4-oxadiazol-3-yl)cyclopropyl)phenyl)-2-(4-(ethylsulfonyl)phenyl)acetamide;
N-(3,5-dichloro-4-(1-(5-(6-methoxypyridin-3-yl)-1,2,4-oxadiazol-3-yl)cyclopropyl) phenyl)-2-(4-(ethylsulfonyl)phenyl)acetamide;
N-(3,5-dichloro-4-(1-(5-(4-fluorobenzyl)-1,2,4-oxadiazol-3-yl)cyclopropyl)phenyl)-2-(4-(ethylsulfonyl)phenyl)acetamide;
N-(3,5-dichloro-4-(1-(5-(4-methoxyphenyl)-1,2,4-oxadiazol-3-yl)cyclopropyl)phenyl)-2-(4-(ethylsulfonyl)phenyl)acetamide;
N-(3,5-dichloro-4-(1-(5-isopropyl-1,2,4-oxadiazol-3-yl)cyclopropyl)phenyl)-2-(4-(ethylsulfonyl)phenyl)acetamide;
N-(4-(1-(1,2,4-oxadiazol-3-yl)cyclopropyl)-3,5-dichlorophenyl)-2-(4-(ethylsulfonyl) phenyl)acetamide;
N-(3,5-dichloro-4-(1-(5-cyclopropyl-1,2,4-oxadiazol-3-yl) cyclopropyl)phenyl)-2-(4-(ethylsulfonyl)phenyl)acetamide;
N-(3,5-dichloro-4-(1-(5-(3,3-difluorocyclobutyl)-1,2,4-oxadiazol-3-yl)cyclopropyl) phenyl)-2-(4-(ethylsulfonyl)phenyl)acetamide;
N-(3,5-dichloro-4-(1-(5-(tetrahydro-2H-pyran-4-yl)-1,2,4-oxadiazol-3-yl)cyclopropyl) phenyl)-2-(4-(ethylsulfonyl)phenyl)acetamide;
N-(3,5-difluoro-4-(1-(5-(4-fluorophenyl)-1,2,4-oxadiazol-3-yl)cyclopropyl)phenyl)-2-(4-(ethylsulfonyl)phenyl)acetamide;
N-(3-chloro-5-fluoro-4-(1-(5-(4-fluorophenyl)-1,2,4-oxadiazol-3-yl)cyclopropyl)phenyl)-2-(4-(ethylsulfonyl)phenyl)acetamide;
N-(3-chloro-4-(1-(5-(4-chlorophenyl)-1,2,4-oxadiazol-3-yl)cyclopropyl)-5-fluorophenyl)-2-(4-(ethylsulfonyl)phenyl)acetamide;
N-(3-chloro-5-fluoro-4-(1-(5-(4-(trifluoromethyl)phenyl)-1,2,4-oxadiazol-3-yl) cyclopropyl)phenyl)-2-(4-(ethylsulfonyl)phenyl)acetamide;
N-(3-chloro-4-(1-(5-(4-cyanophenyl)-1,2,4-oxadiazol-3-yl)cyclopropyl)-5-fluorophenyl)-2-(4-(ethylsulfonyl)phenyl)acetamide;
2-(4-(ethylsulfonyl)phenyl)-N-(4-(1-(5-(4-fluorophenyl)-1,2,4-oxadiazol-3-yl) cyclopropyl)-3,5-dimethylphenyl)acetamide;
N-(3,5-dichloro-4-(1-(5-(4-(trifluoromethoxy)phenyl)-1,2,4-oxadiazol-3-yl) cyclopropyl) phenyl)-2-(4-(ethylsulfonyl)phenyl)acetamide;
N-(3,5-dichloro-4-(1-(3-(4-fluorophenyl)-1,2,4-oxadiazol-5-yl)cyclopropyl)phenyl)-2-(4-(ethylsulfonyl)phenyl)acetamide;
N-(3,5-dichloro-4-(1-(6-fluorobenzo[d]oxazol-2-yl)cyclopropyl) phenyl)-2-(4-(ethylsulfonyl)phenyl)acetamide;
2-(4-(ethylsulfonyl)phenyl)-N-(4-(1-(6-fluorobenzo[d]oxazol-2-yl)cyclopropyl)-3,5-dimethylphenyl)acetamide;
N-(3,5-dichloro-4-(1-(5-(4-fluorophenyl)oxazol-2-yl)cyclopropyl)phenyl)-2-(4-(ethylsulfonyl)phenyl)acetamide;
N-(3,5-dichloro-4-(1-(5-(4-chlorophenyl)oxazol-2-yl)cyclopropyl) phenyl)-2-(4-(ethylsulfonyl)phenyl)acetamide;
N-(3,5-dichloro-4-(1-(5-(3-fluoro-4-methoxyphenyl)oxazol-2-yl)cyclopropyl)phenyl)-2-(4-(ethylsulfonyl)phenyl)acetamide;
N-(3,5-dichloro-4-(1-(5-(4-fluorophenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(4-(trifluoromethyl)phenyl)-1,2,4-oxadiazol-3-yl)cyclopropyl) phenyl)-2-(5-(ethylsulfonyl)pyridin-2-yl)acetamide;
N-(3,5-dichloro-4-(1-(5-(p-tolyl)-1,2,4-oxadiazol-3-yl)cyclopropyl) phenyl)-2-(5-(ethylsulfonyl)pyridin-2-yl)acetamide;
N-(3,5-dichloro-4-(1-(5-(3,4-difluorophenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(3,4,5-trifluorophenyl)-1,2,4-oxadiazol-3-yl)cyclopropyl) phenyl)-2-(5-(ethylsulfonyl)pyridin-2-yl)acetamide;
N-(3,5-dichloro-4-(1-(5-(2-chloro-4-fluorophenyl)-1,2,4-oxadiazol-3-yl)cyclopropyl) phenyl)-2-(5-(ethylsulfonyl)pyridin-2-yl)acetamide;
N-(3,5-dichloro-4-(1-(5-(3-chloro-4-fluorophenyl)-1,2,4-oxadiazol-3-yl)cyclopropyl) phenyl)-2-(5-(ethylsulfonyl)pyridin-2-yl)acetamide;
N-(3,5-dichloro-4-(1-(5-(3,4-dichlorophenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(2,4-dichiorophenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(4-chiorophenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(3-chlorophenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(4-methoxyphenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(4-cyanophenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(5-chlorothiophen-2-yl)-1,2,4-oxadiazol-3-yl)cyclopropyl) phenyl)-2-(5-(ethylsulfonyl)pyridin-2-yl)acetamide;
N-(3,5-dichloro-4-(1-(5-(3,3-difluorocyclobutyl)-1,2,4-oxadiazol-3-yl)cyclopropyl) phenyl)-2-(5-(ethylsulfonyl)pyridin-2-yl)acetamide;
N-(3,5-dichloro-4-(1-(5-cyclopropyl-1,2,4-oxadiazol-3-yl)cyclopropyl)phenyl)-2-(5-(ethylsulfonyl)pyridin-2-yl)acetamide;
2-(5-(ethylsulfonyl)pyridin-2-yl)-N-(4-(1-(5-(4-fluorophenyl)-1,2,4-oxadiazol-3-yl)cyclopropyl)-3,5-dimethylphenyl)acetamide;
N-(3,5-dichloro-4-(1-(6-fluorobenzo[d]oxazol-2-yl)cyclopropyl) phenyl)-2-(5-(ethylsulfonyl)pyridin-2-yl)acetamide;
2-(5-(ethylsulfonyl)pyridin-2-yl)-N-(4-(1-(6-fluorobenzo[d]oxazol-2-yl)cyclopropyl)-3,5-dimethylphenyl)acetamide;
N-(3,5-dichloro-4-(1-(5-(4-fluorophenyl)oxazol-2-yl)cyclopropyl)phenyl)-2-(5-(ethylsulfonyl)pyridin-2-yl)acetamide
N-(3,5-dichloro-4-(1-(5-(4-fluorophenyl)-1,2,4-oxadiazol-3-yl)cyclopropyl)phenyl)-2-(4-(ethylsulfonimidoyl)phenyl)acetamide;
N-(3,5-dichloro-4-(1-(5-(4-(trifluoromethyl)phenyl)-1,2,4-oxadiazol-3-yl)cyclopropyl) phenyl)-2-(4-(ethylsulfonimidoyl)phenyl)acetamide;
2-(4-(N-cyclopropylsulfamoyl)phenyl)-N-(3,5-dichloro-4-(1-(5-(4-fluorophenyl)-1,2,4-oxadiazol-3-yl)cyclopropyl)phenyl)acetamide;
2-(4-(N-cyclopropylsulfamoyl)phenyl)-N-(3,5-dichloro-4-(1-(5-(4-(trifluoromethyl) phenyl)-1,2,4-oxadiazol-3-yl)cyclopropyl)phenyl)acetamide;
2-(4-(N-cyclopropylsulfamoyl)phenyl)-N-(3,5-dichloro-4-(1-(6-fluorobenzo[d]oxazol-2-yl)cyclopropyl)phenyl)acetamide;
N-(3,5-dichloro-4-(1-(5-(4-fluorophenyl)-1,2,4-oxadiazol-3-yl)cyclopropyl)phenyl)-2-(6-(ethylsulfonyl)pyridin-3-yl)acetamide;
N-(3,5-dichloro-4-(1-(5-(4-(trifluoromethyl)phenyl)-1,2,4-oxadiazol-3-yl)cyclopropyl) phenyl)-2-(6-(ethylsulfonyl)pyridin-3-yl)acetamide;
N-(3,5-dichloro-4-(1-(5-(3,4-difluorophenyl)-1,2,4-oxadiazol-3-yl)cyclopropyl)phenyl)-2-(6-(ethylsulfonyl)pyridin-3-yl)acetamide;
N-(3,5-dichloro-4-(1-(5-(2,4-difluorophenyl)-1,2,4-oxadiazol-3-yl)cyclopropyl)phenyl)-2-(6-(ethylsulfonyl)pyridin-3-yl)acetamide;
N-(3,5-dichloro-4-(1-(5-(3-chloro-4-fluorophenyl)-1,2,4-oxadiazol-3-yl)cyclopropyl) phenyl)-2-(6-(ethylsulfonyl)pyridin-3-yl)acetamide;
N-(3,5-dichloro-4-(1-(5-(3,4-dichlorophenyl)-1,2,4-oxadiazol-3-yl)cyclopropyl)phenyl)-2-(6-(ethylsulfonyl)pyridin-3-yl)acetamide;
N-(3,5-dichloro-4-(1-(5-(4-chiorophenyl)-1,2,4-oxadiazol-3-yl)cyclopropyl)phenyl)-2-(6-(ethylsulfonyl)pyridin-3-yl)acetamide;
N-(3,5-dichloro-4-(1-(5-(4-cyanophenyl)-1,2,4-oxadiazol-3-yl)cyclopropyl)phenyl)-2-(6-(ethylsulfonyl)pyridin-3-yl)acetamide;
N-(3,5-dichloro-4-(1-(6-fluorobenzo[d]oxazol-2-yl)cyclopropyl)phenyl)-2-(6-(ethylsulfonyl)pyridin-3-yl)acetamide;
2-(6-(ethylsulfonyl)pyridin-3-yl)-N-(4-(1-(5-(4-fluorophenyl)-1,2,4-oxadiazol-3-yl)cyclopropyl)-3,5-dimethylphenyl)acetamide;
2-(6-(ethylsulfonyl)pyridin-3-yl)-N-(4-(1-(6-fluorobenzo[d]oxazol-2-yl)cyclopropyl)-3,5-dimethylphenyl)acetamide;
N-(3,5-dichloro-4-(1-(5-(4-fluorophenyl)-1,2,4-oxadiazol-3-yl)cyclopropyl)phenyl)-2-(5-(ethylsulfonyl)pyrazin-2-yl)acetamide;
N-(3,5-dichloro-4-(1-(6-fluorobenzo[d]oxazol-2-yl)cyclopropyl) phenyl)-2-(5-(ethylsulfonyl)pyrazin-2-yl)acetamide;
N-(3,5-dichloro-4-(1-(5-(4-fluorophenyl)-1,3,4-oxadiazol-2-yl)cyclopropyl)phenyl)-2-(4-(ethylsulfonyl)phenyl)acetamide;
N-(3,5-dichloro-4-(1-(5-(4-fluorophenyl)-1,3,4-oxadiazol-2-yl)cyclopropyl)phenyl)-2-(5-(ethylsulfonyl)pyridin-2-yl)acetamide;
N-(3,5-dichloro-4-(1-(5-(4-fluorophenyl)-1,2,4-oxadiazol-3-yl)cyclopropyl)phenyl)-2-(4-(methylsulfonyl)phenyl)acetamide;
N-(3,5-dichloro-4-(1-(5-(4-chiorophenyl)-1,2,4-oxadiazol-3-yl)cyclopropyl)phenyl)-2-(4-(methylsulfonyl)phenyl)acetamide;
N-(3,5-dichloro-4-(1-(5-(4-(trifluoromethyl)phenyl)-1,2,4-oxadiazol-3-yl)cyclopropyl) phenyl)-2-(4-(methylsulfonyl)phenyl)acetamide;
N-(3,5-dichloro-4-(1-(5-(4-cyanophenyl)-1,2,4-oxadiazol-3-yl) cyclopropyl)phenyl)-2-(4-(methylsulfonyl)phenyl)acetamide;
N-(3,5-dichloro-4-(1-(5-(3-chloro-4-fluorophenyl)-1,2,4-oxadiazol-3-yl)cyclopropyl) phenyl)-2-(4-(methylsulfonyl)phenyl)acetamide;
N-(3,5-dichloro-4-(1-(6-fluorobenzo[d]oxazol-2-yl) cyclopropyl) phenyl)-2-(4-(methylsulfonyl)phenyl)acetamide;
N-(3,5-dichloro-4-(1-(3-(4-chiorophenyl)-1,2,4-oxadiazol-5-yl) cyclopropyl)phenyl)-2-(4-(ethylsulfonyl)phenyl)acetamide;
N-(3,5-dichloro-4-(1-(3-(4-(trifluoromethyl)phenyl)-1,2,4-oxadiazol-5-yl)cyclopropyl) phenyl)-2-(4-(ethylsulfonyl)phenyl)acetamide;
N-(3,5-dichloro-4-(1-(3-(4-cyanophenyl)-1,2,4-oxadiazol-5-yl) cyclopropyl)phenyl)-2-(4-(ethylsulfonyl)phenyl)acetamide;
N-(3,5-dichloro-4-(1-(3-(3,4-difluorophenyl)-1,2,4-oxadiazol-5-yl) cyclopropyl)phenyl)-2-(4-(ethylsulfonyl)phenyl)acetamide;
N-(3,5-dichloro-4-(1-(3-(3-chloro-4-fluorophenyl)-1,2,4-oxadiazol-5-yl)cyclopropyl) phenyl)-2-(4-(ethylsulfonyl)phenyl)acetamide;
N-(3,5-dichloro-4-(1-(3-(2,4-difluorophenyl)-1,2,4-oxadiazol-5-yl) cyclopropyl)phenyl)-2-(4-(ethylsulfonyl)phenyl)acetamide;
N-(3,5-dichloro-4-(1-(3-(p-tolyl)-1,2,4-oxadiazol-5-yl) cyclopropyl) phenyl)-2-(4-(ethylsulfonyl)phenyl)acetamide;
N-(3,5-dichloro-4-(1-(3-phenyl-1,2,4-oxadiazol-5-yl)cyclopropyl) phenyl)-2-(4-(ethylsulfonyl)phenyl)acetamide;
N-(3,5-dichloro-4-(1-(3-cyclopropyl-1,2,4-oxadiazol-5-yl) cyclopropyl)phenyl)-2-(4-(ethylsulfonyl)phenyl)acetamide;
N-(3,5-dichloro-4-(1-(3-(3,3-difluorocyclobutyl)-1,2,4-oxadiazol-5-yl) cyclopropyl) phenyl)-2-(4-(ethylsulfonyl)phenyl)acetamide;
N-(3,5-dichloro-4-(1-(3-(2-fluoro-4-methoxyphenyl)-1,2,4-oxadiazol-5-yl) cyclopropyl) phenyl)-2-(4-(ethylsulfonyl)phenyl)acetamide;
2-(4-(ethylsulfonyl)phenyl)-N-(4-(1-(3-(4-fluorophenyl)-1,2,4-oxadiazol-5-yl) cyclopropyl)-3,5-dimethylphenyl)acetamide;
2-(4-(ethylsulfonyl)phenyl)-N-(4-(1-(5-(4-fluorophenyl)oxazol-2-yl)cyclopropyl)-3,5-dimethylphenyl)acetamide;
N-(3,5-dichloro-4-(1-(5-(4-chlorophenyl)-1,2,4-oxadiazol-3-yl) cyclopropyl)phenyl)-2-(4-(ethylsulfonimidoyl)phenyl)acetamide;
N-(3,5-dichloro-4-(1-(6-fluorobenzo[d]oxazol-2-yl) cyclopropyl) phenyl)-2-(4-(ethylsulfonimidoyl)phenyl)acetamide;
N-(3,5-dichloro-4-(1-(6-chlorobenzo[d]oxazol-2-yl) cyclopropyl) phenyl)-2-(4-(ethylsulfonimidoyl)phenyl)acetamide;
N-(4-(1-(benzo[d]oxazol-2-yl)cyclopropyl)-3,5-dichlorophenyl)-2-(4-(ethylsulfonyl) phenyl)acetamide;
N-(3,5-dichloro-4-(1-(6-chlorobenzo[d]oxazol-2-yl)cyclopropyl) phenyl)-2-(4-(ethylsulfonyl)phenyl)acetamide;
N-(3,5-dichloro-4-(1-(6-(trifluoromethyl)benzo[d]oxazol-2-yl)cyclopropyl)phenyl)-2-(4-(ethylsulfonyl)phenyl)acetamide;
N-(4-(1-(benzo[d]oxazol-2-yl)cyclopropyl)-3,5-dichlorophenyl)-2-(5-(ethylsulfonyl) pyridin-2-yl)acetamide;
N-(3,5-dichloro-4-(1-(6-chlorobenzo[d]oxazol-2-yl)cyclopropyl) phenyl)-2-(5-(ethylsulfonyl)pyridin-2-yl)acetamide;
N-(3,5-dichloro-4-(1-(6-(trifluoromethyl)benzo[d]oxazol-2-yl)cyclopropyl)phenyl)-2-(5-(ethylsulfonyl)pyridin-2-yl)acetamide;
N-(4-(1-(6-chlorobenzo[d]oxazol-2-yl)cyclopropyl)-3,5-dimethylphenyl)-2-(5-(ethylsulfonyl)pyridin-2-yl)acetamide;
N-(3,5-dichloro-4-(1-(5-(4-chiorophenyl)-1,3,4-oxadiazol-2-yl)cyclopropyl)phenyl)-2-(4-(ethylsulfonyl)phenyl)acetamide;
N-(3,5-dichloro-4-(1-(5-(4-(trifluoromethyl)phenyl)-1,3,4-oxadiazol-2-yl)cyclopropyl)phenyl)-2-(4-(ethylsulfonyl)phenyl)acetamide;
N-(4-(1-(5-(4-chlorophenyl)-1,3,4-oxadiazol-2-yl)cyclopropyl)-3,5-dimethylphenyl)-2-(4-(ethylsulfonyl)phenyl)acetamide;
2-(4-(N-cyclopropylsulfamoyl)phenyl)-N-(3,5-dichloro-4-(1-(5-(4-chlorophenyl)-1,2,4-oxadiazol-3-yl)cyclopropyl)phenyl)acetamide;
2-(4-(N-cyclopropylsulfamoyl)phenyl)-N-(3,5-dichloro-4-(1-(6-chlorobenzo[d]oxazol-2-yl)cyclopropyl)phenyl)acetamide;
N-(3,5-dichloro-4-(1-(5-(4-cyclopropyiphenyl)-1,2,4-oxadiazol-3-yl)cyclopropyl) phenyl)-2-(4-(ethylsulfonyl)phenyl)acetamide;
N-(4-(1-(5-(4-chlorophenyl)-1,2,4-oxadiazol-3-yl)cyclopropyl)-3,5-difluorophenyl)-2-(4-(ethylsulfonyl)phenyl)acetamide;
N-(3,5-difluoro-4-(1-(5-(4-(trifluoromethyl)phenyl)-1,2,4-oxadiazol-3-yl)cyclopropyl) phenyl)-2-(4-(ethylsulfonyl)phenyl)acetamide;
N-(4-(1-(5-(4-cyanophenyl)-1,2,4-oxadiazol-3-yl)cyclopropyl)-3,5-difluorophenyl)-2-(4-(ethylsulfonyl)phenyl)acetamide;
N-(4-(1-(5-(4-chlorophenyl)-1,2,4-oxadiazol-3-yl)cyclopropyl)-3,5-dimethylphenyl)-2-(4-(ethylsulfonyl)phenyl)acetamide;
N-(3,5-dimethyl-4-(1-(5-(4-(trifluoromethyl)phenyl)-1,2,4-oxadiazol-3-yl)cyclopropyl) phenyl)-2-(4-(ethylsulfonyl)phenyl)acetamide;
N-(4-(1-(5-(4-cyanophenyl)-1,2,4-oxadiazol-3-yl)cyclopropyl)-3,5-dimethylphenyl)-2-(4-(ethylsulfonyl)phenyl)acetamide.

The novel compounds of this invention may be prepared using the reactions and techniques as shown in scheme below and described in this section. The reactions are performed in solvents appropriate to the reagents and materials employed and are suitable for the transformations being affected. It is understood by those skilled in the art that the nature and order of the synthetic steps presented may be varied for the purpose of optimizing the formation of the compounds of the present invention. It will also be well appreciated that one or more of the reactants may be protected and deprotected for facile synthesis by techniques known to persons skilled in the art. It will also be appreciated that one or more of the compounds of the present invention may exist in stereoisomeric and/or diastereomeric forms. Such stereoisomers and/or diastereoisomers as well as their optical antipodes are to be construed to be within the scope of the present invention. It will also be well appreciated that one or more of these compounds may be converted to their salts and other derivatives based on the specific groups present on the compounds, which can be well comprehended by persons skilled in the art.

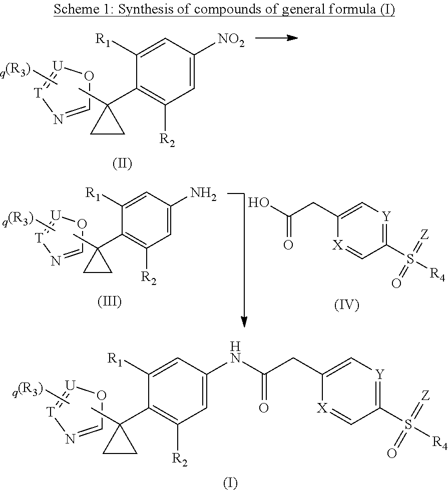

The compound (II) can be obtained using general techniques described in literature for carbocyclic/heterocyclic ring generation for e.g. in WO2005034837, WO2015140130. The compound (III) can be obtained by reduction of nitro group by using general nitro group reduction techniques described in the literature. Preferred methods involve reduction using stannous chloride and catalytic hydrogenation in solvents like methanol, THF, etc.

The compounds of general formula (IV) can be obtained by several methods described in the literature for e.g. in Bioorganic & Medicinal Chemistry Letters 2011, 21 (5), 1549 and WO2015082533. The compounds of the general formula (I) can be obtained by coupling of (III) and (IV) or sodium salt of (IV) using various amide bond formation techniques as described in Tetrahedron 2005, 61, 10827.

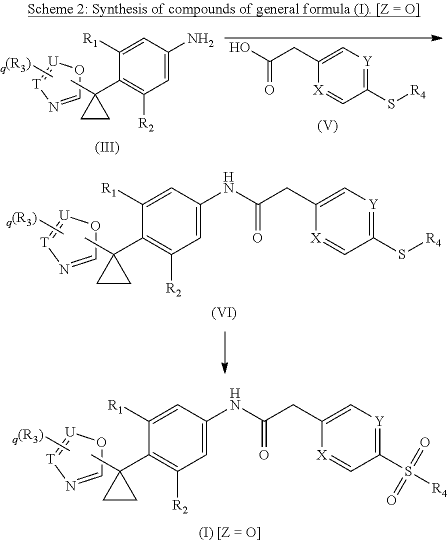

Alternatively, the compounds of the general formula (I) can also be prepared by scheme 2. The compounds (VI) can be obtained by coupling of (III) and (V) by following the process of scheme 1. The compounds of the general formula (I) are then obtained by oxidation of (VI) using various sulfur oxidation techniques available in literature. Preferred method involves oxidation with oxone in aq. acetone.

The invention is explained in greater detail by the examples given below, which are provided by way of illustration only and therefore should not be construed to limit the scope of the invention.

The ¹H NMR spectra were recorded on a Brucker Avance-400 spectrometer (400 MHz). The chemical shifts (6) are reported in parts per million (ppm) relative to Tetramethyl silane (TMS), in either CDCl₃ or DMSO-d₆ solution. Mass spectra (ESI-MS) were obtained on Shimadzu LC-MS 2010-A spectrometer.

### List of Abbreviations

CH₃CN: Acetonitrile
CDCl₃: Deuterated chloroform
Cs₂CO₃: Cesium carbonate
DCE: Dichloro ethane
DIPEA: Disopropyl ethyl amine
DMF: Dimethyl formamide
DCM: Dichloromethane
DIBAL-H: Diisobutylaluminium hydride
DMSO: Dimethyl sulfoxide
DMSO-d₆: Hexadeuterodimethyl sulfoxide
EDC.HCl: N-(3-Dimethyl aminopropyl)-N′-ethyl carbodiimide hydrochloride
EtOH: Ethanol
EtOAc: Ethyl acetate
HOBT: 1-Hydroxy benzotriazole
HCl: Hydrochloric acid
K₂CO₃: Potassium carbonate
LiOH.H₂O: Lithium hydroxide monohydrate
MeOH: Methanol
Na₂SO₄: Sodium sulfate
NaH: Sodium Hydride
NaHCO₃: Sodium bicarbonate
NaOH: Sodium hydroxide
NH₄Cl: Ammonium chloride
POCl₃: Phosphoryl chloride
SnCl₂.2H₂O: Stannous chloride dihydrate
TEA: Triethyl amine
TFA: Trifluoroacetic acid
THF: Tetrahydrofuran
¹H NMR: Proton Nuclear Magnetic Resonance
h: Hour(s)
RT: room temperature [25-30° C.]
min: Minute(s)
J: Coupling constant in units of Hz
Hz: Hertz

### Preparation of Intermediates of Formula (III)

### Preparation of 3,5-dichloro-4-(1-(5-(4-fluorophenyl)-1,2,4-oxadiazol-3-yl) cyclopropyl)aniline (III-1)

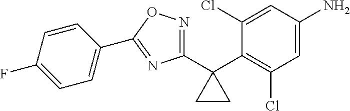 [SMILES: Nc1cc(Cl)c(C2(c3noc(-c4ccc(F)cc4)n3)CC2)c(Cl)c1]

### Step 1: ethyl 2-cyano-2-(2,6-dichloro-4-nitrophenyl)acetate

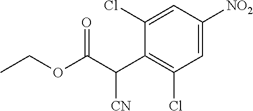 [SMILES: CCOC(=O)C(C#N)c1c(Cl)cc([N+](=O)[O-])cc1Cl]

To a stirring solution of 1,2,3-trichloro-5-nitrobenzene (50 g, 221 mmol) and Cs₂CO₃ (151 g, 464 mmol) in DMF (200 mL) was added ethyl cyano acetate (28.3 mL, 265 mmol) at 10-20° C. Reaction mixture was stirred at RT for 1 h, before it was cooled and poured in to 200 ml dil. HCl solution. Solid obtained was filtered to get title product as brown solid. ¹H NMR (DMSO-d₆): 8.47 (s, 2H), 6.54 (s, 1H), 4.28 (q, J=6.8 Hz, 2H), 1.23 (t, J=6.8 Hz, 3H).

### Step 2: 2-(2,6-dichloro-4-nitrophenyl)acetonitrile

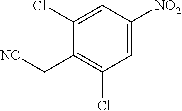 [SMILES: N#CCc1c(Cl)cc([N+](=O)[O-])cc1Cl]

To a stirring solution of product of step 1 (52.0 g, 172 mmol) in DMSO (12 ml) and water (4.5 ml) was added lithium chloride (9.46 g, 223 mmol) at RT. Reaction mixture was heated at 165° C. for 1 h before it was cooled and poured in to ice cold water. Solid obtained was filtered to get 25 g of title product. ¹H NMR (DMSO-d₆): 8.42 (s, 2H), 4.31 (s, 2H).

### Step 3: 1-(2,6-dichloro-4-nitrophenyl)cyclopropane-1-carbonitrile

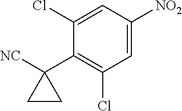 [SMILES: N#CC1(c2c(Cl)cc([N+](=O)[O-])cc2Cl)CC1]

To a stirring solution of product of step 2 (4.0 g, 17.31 mmol) in CH₃CN (40 ml) was added ethylene dibromide (4.48 ml, 51.9 mmol) followed by tetrabutyl ammonium bromide (5.58 g, 17.31 mmol). To this was added 8 ml 50% NaOH solution at RT and reaction mixture was stirred at 70-75° C. for 12 h. Reaction mixture was then poured in to dil. HCl (100 mL) and extracted with EtOAc. Organic layer was separated, washed with water, dried over Na₂SO₄ and distilled out to get crude product which was purified by column chromatography (4% EtOAc in Hexane) to get title product. ¹H NMR (DMSO-d₆): 8.42 (s, 2H), 2.06-2.03 (m, 2H), 1.57-1.53 (m, 2H).

### Step 4: 1-(2,6-dichloro-4-nitrophenyl)-N′-hydroxycyclopropane-1-carboximidamide

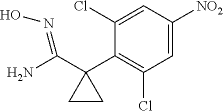 [SMILES: N/C(=N\O)C1(c2c(Cl)cc([N+](=O)[O-])cc2Cl)CC1]

To a stirring solution of product of step 3 (5 g, 19.45 mmol) in rectified spirit (50 ml) was added hydroxyl amine hydrochloride (3.38 g, 48.6 mmol) and K₂CO₃ (6.72 g, 48.6 mmol) at RT. Reaction mixture was refluxed for 16 h. Reaction mixture was diluted with water and precipitated solid was filtered to get title product. ¹H NMR (DMSO-d₆): 9.26 (s, 1H), 8.19 (s, 2H), 5.16 (s, 2H), 1.74-1.70 (m, 2H), 1.08-1.05 (m, 2H).

### Step 5: 3-(1-(2,6-dichloro-4-nitrophenyl)cyclopropyl)-5-(4-fluorophenyl)-1,2,4-oxadiazole

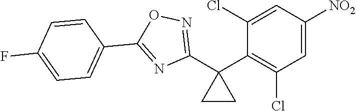 [SMILES: O=[N+]([O-])c1cc(Cl)c(C2(c3noc(-c4ccc(F)cc4)n3)CC2)c(Cl)c1]

To a stirring solution of 4-fluorobenzoic acid (0.560 g, 4 mmol), HOBT (0.756 g, 5.60 mmol) in DMF (30 mL) was added EDC.HCl (1.073 g, 5.60 mmol) and stirred for 15 min. at RT. To this was added product of step 4 (1.16 g, 4 mmol) and stirred at 110° C. for 16 h. Reaction mixture was poured in water and extracted with EtOAc. Organic layer was washed with water followed by NaHCO₃ solution, dried over Na₂SO₄ and distilled out to get crude product which was column purified (3% EtOAc in Hexane) to get title product. ¹H NMR (DMSO-d₆): 8.38 (s, 2H), 8.13 (dd, J=5.2 & 8.8 Hz, 2H), 7.46 (t, 2H), 2.01 (bd, 2H), 1.65 (bd, 2H).

### Step 6: 3,5-dichloro-4-(1-(5-(4-fluorophenyl)-1,2,4-oxadiazol-3-yl)cyclopropyl)aniline

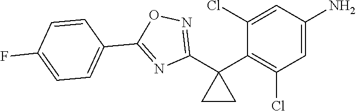 [SMILES: Nc1cc(Cl)c(C2(c3noc(-c4ccc(F)cc4)n3)CC2)c(Cl)c1]

To a stirring solution of product of step 5 (140 mg, 0.355 mmol) in EtOAc (5 ml) was added SnCl₂.2H₂O (401 mg, 1.776 mmol) and stirred at RT for 3 h. Reaction mixture was diluted with EtOAc, basified with aq. ammonia and passed through Hyflo bed. Organic layer was separated, dried over Na₂SO₄ and distilled out to get title product. ¹H NMR (DMSO-d₆): 8.12-8.08 (m, 2H), 7.46-7.42 (m, 2H), 6.63 (s, 2H), 5.72 (s, 2H), 1.82-1.79 (m, 2H), 1.45-1.42 (m, 2H). ESI-MS (m/z): 364.20 (M+H)⁺.

Using appropriate starting materials and suitable modifications of the process described for the preparation of intermediate III-1, including suitable addition and/or deletion of steps as may be necessary, well within the scope of a person skilled in the art, the following intermediates (Table 2) were prepared in an analogues manner.

TABLE 2

| Intermediate | Structure | ESI-MS (m/z): (M + H)⁺ |
|---|---|---|
| III-2 | 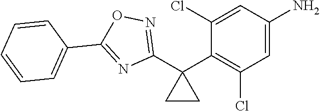 [SMILES: Nc1cc(Cl)c(C2(c3noc(-c4ccccc4)n3)CC2)c(Cl)c1] | 345.90 |
| III-3 | 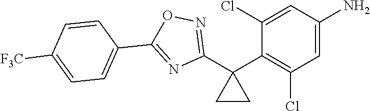 [SMILES: Nc1cc(Cl)c(C2(c3noc(-c4ccc(C(F)(F)F)cc4)n3)CC2)c(Cl)c1] | 413.95 |
| III-4 | 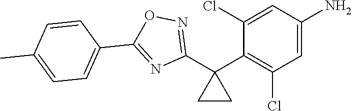 [SMILES: Cc1ccc(-c2nc(C3(c4c(Cl)cc(N)cc4Cl)CC3)no2)cc1] | 360.05 |
| III-5 | 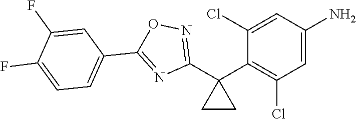 [SMILES: Nc1cc(Cl)c(C2(c3noc(-c4ccc(F)c(F)c4)n3)CC2)c(Cl)c1] | 382.00 |
| III-6 | 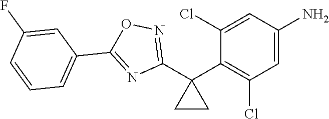 [SMILES: Nc1cc(Cl)c(C2(c3noc(-c4cccc(F)c4)n3)CC2)c(Cl)c1] | 364.00 |
| III-7 | 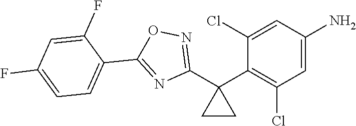 [SMILES: Nc1cc(Cl)c(C2(c3noc(-c4ccc(F)cc4F)n3)CC2)c(Cl)c1] | 381.95 |
| III-8 | 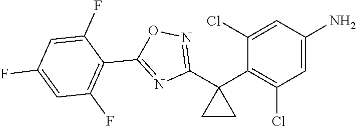 [SMILES: Nc1cc(Cl)c(C2(c3noc(-c4c(F)cc(F)cc4F)n3)CC2)c(Cl)c1] | 400.00 |
| III-9 | 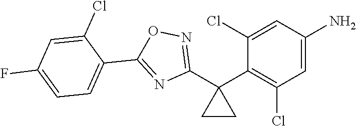 [SMILES: Nc1cc(Cl)c(C2(c3noc(-c4ccc(F)cc4Cl)n3)CC2)c(Cl)c1] | 397.90 |
| III-10 | 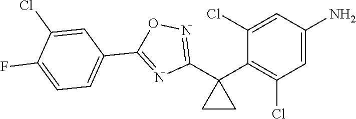 [SMILES: Nc1cc(Cl)c(C2(c3noc(-c4ccc(F)c(Cl)c4)n3)CC2)c(Cl)c1] | 398.00 |
| III-11 | 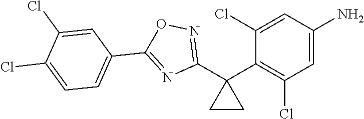 [SMILES: Nc1cc(Cl)c(C2(c3noc(-c4ccc(Cl)c(Cl)c4)n3)CC2)c(Cl)c1] | 415.95 |
| III-12 | 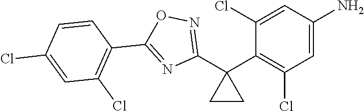 [SMILES: Nc1cc(Cl)c(C2(c3noc(-c4ccc(Cl)cc4Cl)n3)CC2)c(Cl)c1] | 415.95 |
| III-13 | 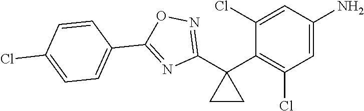 [SMILES: Nc1cc(Cl)c(C2(c3noc(-c4ccc(Cl)cc4)n3)CC2)c(Cl)c1] | 379.95 |
| III-14 | 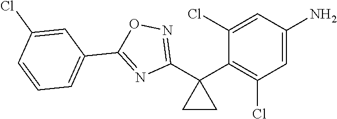 [SMILES: Nc1cc(Cl)c(C2(c3noc(-c4cccc(Cl)c4)n3)CC2)c(Cl)c1] | 379.95 |
| III-15 | 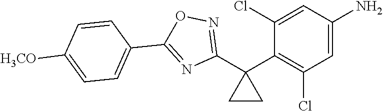 [SMILES: COc1ccc(-c2nc(C3(c4c(Cl)cc(N)cc4Cl)CC3)no2)cc1] | 376.00 |
| III-16 | 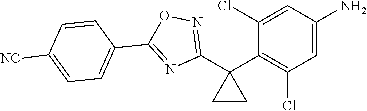 [SMILES: N#Cc1ccc(-c2nc(C3(c4c(Cl)cc(N)cc4Cl)CC3)no2)cc1] | 370.95 |
| III-17 | 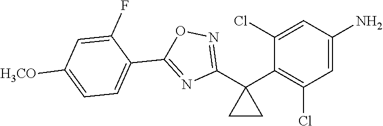 [SMILES: COc1ccc(-c2nc(C3(c4c(Cl)cc(N)cc4Cl)CC3)no2)c(F)c1] | 394.05 |
| III-18 | 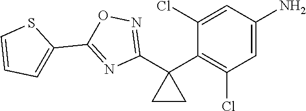 [SMILES: Nc1cc(Cl)c(C2(c3noc(-c4cccs4)n3)CC2)c(Cl)c1] | 352.20 |
| III-19 | 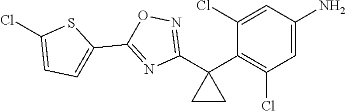 [SMILES: Nc1cc(Cl)c(C2(c3noc(-c4ccc(Cl)s4)n3)CC2)c(Cl)c1] | 385.95 |
| III-20 | 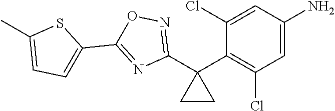 [SMILES: Cc1ccc(-c2nc(C3(c4c(Cl)cc(N)cc4Cl)CC3)no2)s1] | 366.00 |
| III-21 | 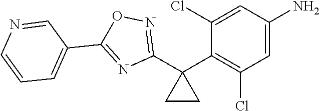 [SMILES: Nc1cc(Cl)c(C2(c3noc(-c4cccnc4)n3)CC2)c(Cl)c1] | 347.00 |
| III-22 | 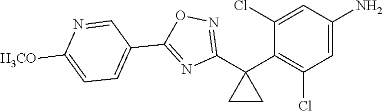 [SMILES: COc1ccc(-c2nc(C3(c4c(Cl)cc(N)cc4Cl)CC3)no2)cn1] | 377.00 |
| III-23 | 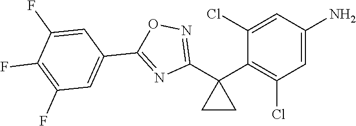 [SMILES: Nc1cc(Cl)c(C2(c3noc(-c4cc(F)c(F)c(F)c4)n3)CC2)c(Cl)c1] | 399.95 |
| III-24 | 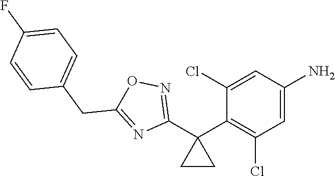 [SMILES: Nc1cc(Cl)c(C2(c3noc(Cc4ccc(F)cc4)n3)CC2)c(Cl)c1] | 378.00 |
| III-25 | 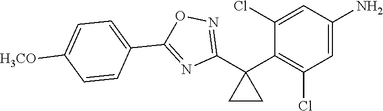 [SMILES: COc1ccc(-c2nc(C3(c4c(Cl)cc(N)cc4Cl)CC3)no2)cc1] | 376.00 |
| III-26 | 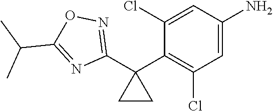 [SMILES: CC(C)c1nc(C2(c3c(Cl)cc(N)cc3Cl)CC2)no1] | 312.00 |
| III-27 | 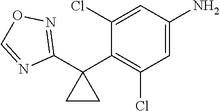 [SMILES: Nc1cc(Cl)c(C2(c3ncon3)CC2)c(Cl)c1] | 269.95 |
| III-28 | 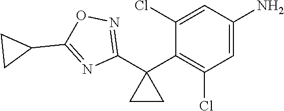 [SMILES: Nc1cc(Cl)c(C2(c3noc(C4CC4)n3)CC2)c(Cl)c1] | 310.10 |
| III-29 | 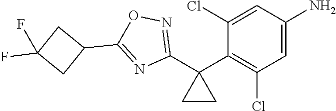 [SMILES: Nc1cc(Cl)c(C2(c3noc(C4CC(F)(F)C4)n3)CC2)c(Cl)c1] | 360.05 |
| III-30 | 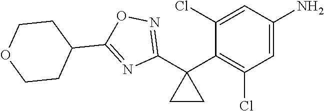 [SMILES: Nc1cc(Cl)c(C2(c3noc(C4CCOCC4)n3)CC2)c(Cl)c1] | 353.90 |
| III-31 | 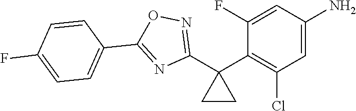 [SMILES: Nc1cc(F)c(C2(c3noc(-c4ccc(F)cc4)n3)CC2)c(Cl)c1] | 332.00 |
| III-32 | 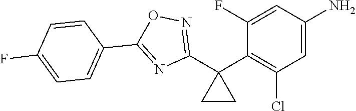 [SMILES: Nc1cc(F)c(C2(c3noc(-c4ccc(F)cc4)n3)CC2)c(Cl)c1] | 347.95 |
| III-33 | 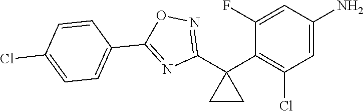 [SMILES: Nc1cc(F)c(C2(c3noc(-c4ccc(Cl)cc4)n3)CC2)c(Cl)c1] | 364.10 |
| III-34 | 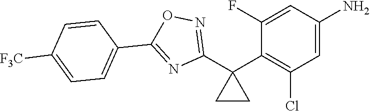 [SMILES: Nc1cc(F)c(C2(c3noc(-c4ccc(C(F)(F)F)cc4)n3)CC2)c(Cl)c1] | 398.05 |
| III-35 | 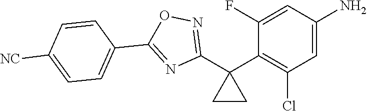 [SMILES: N#Cc1ccc(-c2nc(C3(c4c(F)cc(N)cc4Cl)CC3)no2)cc1] | 354.95 |
| III-36 | 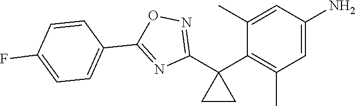 [SMILES: Cc1cc(N)cc(C)c1C1(c2noc(-c3ccc(F)cc3)n2)CC1] | 324.10 |
| III-37 | 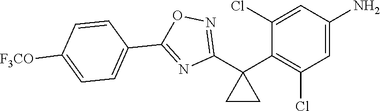 [SMILES: Nc1cc(Cl)c(C2(c3noc(-c4ccc(OC(F)(F)F)cc4)n3)CC2)c(Cl)c1] | — |

### Preparation of 3,5-dichloro-4-(1-(3-(4-fluorophenyl)-1,2,4-oxadiazol-5-yl) cyclopropyl)aniline (III-38)

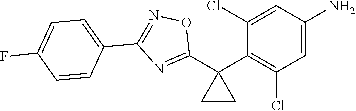 [SMILES: Nc1cc(Cl)c(C2(c3nc(-c4ccc(F)cc4)no3)CC2)c(Cl)c1]

### Step 1: 4-fluoro-N′-hydroxybenzimidamide

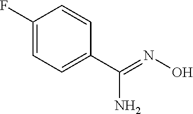 [SMILES: N/C(=N\O)c1ccc(F)cc1]

To a stirring suspension of 4-fluorobenzonitrile (2.1 g, 17.34 mmol) and K₂CO₃ (5.99 g, 43.3 mmol) in 20 ml rectified spirit was added hydroxylamine hydrochloride (3.01 g, 43.3 mmol) at RT. Reaction mixture was refluxed for 12 h. Reaction mixture was poured in water and obtained solid was filtered to get title product. ¹H NMR (DMSO-d₆): 9.62 (s, 1H), 7.72-7.68 (m, 2H), 7.20 (t, 2H), 5.83 (s, 2H).

### Step 2: 5-(1-(2,6-dichloro-4-nitrophenyl)cyclopropyl)-3-(4-fluorophenyl)-1,2,4-oxadiazole

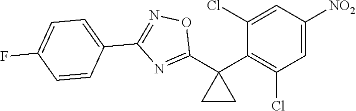 [SMILES: O=[N+]([O-])c1cc(Cl)c(C2(c3nc(-c4ccc(F)cc4)no3)CC2)c(Cl)c1]

Prepared using 1-(2,6-dichloro-4-nitrophenyl)cyclopropane-1-carboxylic acid and product of step 1 by following similar procedure as described for step 5 in intermediate III-1. ¹H NMR (DMSO-d₆): 8.43 (s, 2H), 7.97-7.93 (m, 2H), 7.37 (t, 2H), 2.24-2.21 (m, 2H), 1.87-1.83 (m, 2H).

### Step 3: 3,5-dichloro-4-(1-(3-(4-fluorophenyl)-1,2,4-oxadiazol-5-yl)cyclopropyl)aniline

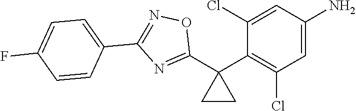 [SMILES: Nc1cc(Cl)c(C2(c3nc(-c4ccc(F)cc4)no3)CC2)c(Cl)c1]

Prepared using product of step 2 by following similar procedure as described for step 6 in intermediate III-1. ¹H NMR (DMSO-d₆): 7.98-7.94 (m, 2H), 7.38-7.34 (m, 2H), 6.67 (s, 2H), 5.83 (s, 2H), 2.04-2.00 (m, 2H), 1.67-1.63 (m, 2H). ESI-MS (m/z): 364.00 (M+H)⁺.

### Preparation of 3,5-dichloro-4-(1-(6-fluorobenzo[d]oxazol-2-yl)cyclopropyl)aniline (III-39)

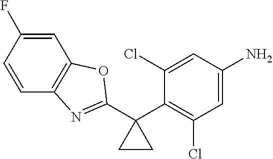 [SMILES: Nc1cc(Cl)c(C2(c3nc4ccc(F)cc4o3)CC2)c(Cl)c1]

### Step 1: 1-(2,6-dichloro-4-nitrophenyl)cyclopropane-1-carbaldehyde

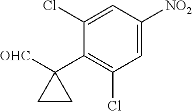 [SMILES: O=CC1(c2c(Cl)cc([N+](=O)[O-])cc2Cl)CC1]

To a stirring solution of 1-(2,6-dichloro-4-nitrophenyl)cyclopropane-1-carbonitrile (4.5 g, 17.50 mmol, prepared in step 3, III-1) in toluene (20 ml) cooled at −60 to −70° C. was added DIBAL-H (16.34 ml, 24.51 mmol) and reaction mixture was stirred at −10 to −20° C. for 1 h. Reaction mixture was quenched with dil. HCl and toluene layer was separated. Reaction mixture was further extracted with DCM. Combine organic layer was distilled out to get crude product which was column purified (5% EtOAc in Hexane) to get title product as red solid. ¹H NMR (DMSO-d₆): 8.69 (s, 1H), 8.30 (s, 2H), 2.05-2.02 (m, 2H), 1.62-1.59 (m, 2H).

### Step 2: 1-(2,6-dichloro-4-nitrophenyl)cyclopropane-1-carboxylic acid

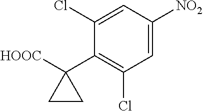 [SMILES: O=C(O)C1(c2c(Cl)cc([N+](=O)[O-])cc2Cl)CC1]

To a stirring solution of product of step 1 (500 mg, 1.92 mmol) in acetone (10 ml) cooled at 0° C. was added 0.5 mL jones reagent and stirred for 3 h at RT. Reaction mixture was poured in water and extracted with EtOAc. Organic layer was distilled out to get title product. ¹H NMR (DMSO-d₆): 8.29 (s, 2H), 1.77-1.80 (m, 2H), 1.31-1.34 (m, 2H).

### Step 3: 1-(2,6-dichloro-4-nitrophenyl)-N-(4-fluoro-2-hydroxyphenyl)cyclopropane-1-carboxamide

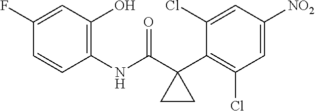 [SMILES: O=C(Nc1ccc(F)cc1O)C1(c2c(Cl)cc([N+](=O)[O-])cc2Cl)CC1]

To a stirring solution of 2-amino-5-fluorophenol (1.03 g, 8.15 mmol) in THF (10 mL) was added TEA (3.3 g, 32.6 mmol) at 5-10° C. To this was added 1-(2,6-dichloro-4-nitrophenyl)cyclopropanecarbonyl chloride (1.5 g, 5.43 mmol, prepared from product of step 2 and oxalyl chloride in THF) at 5-10° C. Reaction mixture was stirred at 10° C. for 1 h. After complete conversion of starting material reaction mixture was diluted with water. The mixture was extracted with EtOAc. Organic layer was washed with brine, dried over Na₂SO₄, and filtered. Removal of the solvent under reduced pressure gave product as a brown oil which was further purified by column chromatography (20% EtOAc in Hexane) to get white solid as pure product. ¹H NMR (DMSO-d₆): 10.18 (s, 1H), 8.40 (s, 2H), 8.0 (s, 1H), 7.53-7.49 (m, 1H), 6.60-6.55 (dd, J=2.8 &10 Hz, 2H), 1.87-1.85 (m, 2H), 1.31-1.29 (m, 2H).

### Step 4: 2-(1-(2,6-dichloro-4-nitrophenyl)cyclopropyl)-6-fluorobenzo[d]oxazole

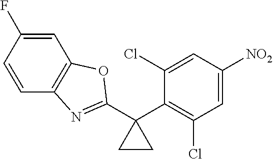 [SMILES: O=[N+]([O-])c1cc(Cl)c(C2(c3nc4ccc(F)cc4o3)CC2)c(Cl)c1]

To a stirring solution of product of step 3 (1.5 g, 3.89 mmol) in toluene (5 v/w) was added p-toluene sulfonic acid monohydrate (1.0 g, 5.84 mmol). The mixture was stirred at reflux temperature for 10 h with continuous removal of water by using Dean-Stark apparatus. After complete conversion of starting material reaction mixture was diluted with water. The mixture was extracted with EtOAc. Organic layer was washed with saturated solution of NaHCO₃, dried over Na₂SO₄, and filtered. Removal of the solvent under reduced pressure gave product as a brown oil which was further purified by column chromatography (20% EtOAc in Hexane) to get off-white solid as pure product. ¹H NMR (CDCl₃): 8.28 (s, 2H), 7.54-7.51 (m, 1H), 7.17-7.14 (dd, J=2.4 & 8 Hz, 1H), 7.06-7.01 (m, 1H), 2.26-2.248 (m, 2H), 1.68-1.65 (m, 2H).

### Step 5: 3,5-dichloro-4-(1-(6-fluorobenzo[d]oxazol-2-yl)cyclopropyl)aniline

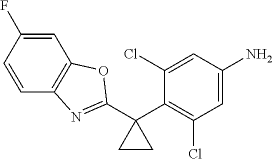 [SMILES: Nc1cc(Cl)c(C2(c3nc4ccc(F)cc4o3)CC2)c(Cl)c1]

To a solution of product of step 4 (0.530 g, 1.44 mmol) in EtOH (10 v/w) was added SnCl₂.2H₂O (1.62, 7.22 mmol) at RT. Reaction mixture was heated to 70-75° C. and stirred for 2 h. After complete conversion of starting material reaction mixture was diluted with EtOAc and basified with ammonia solution. Organic layer was decanted, dried over Na₂SO₄, and filtered. Removal of the solvent under reduce pressure gave product as a yellow solid. ¹H NMR (DMSO-d₆): 7.63-7.57 (m, 2H), 7.20-7.15 (m, 1H) 6.66 (s, 2H), 5.78 (s, 2H), 1.99-1.95 (m, 2H), 1.56-1.52 (m, 2H). ESI-MS (m/z): 336.85 (M+H)⁺.

### Preparation of 4-(1-(6-fluorobenzo[d]oxazol-2-yl)cyclopropyl)-3,5-dimethylaniline (III-40)

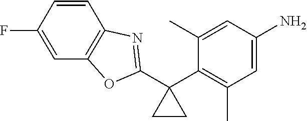 [SMILES: Cc1cc(N)cc(C)c1C1(c2nc3ccc(F)cc3o2)CC1]

Prepared using similar procedure as described for 111-39. ESI-MS (m/z): 297.12 (M+H)⁺.

### Preparation of 3,5-dichloro-4-(1-(5-(4-fluorophenyl)oxazol-2-yl)cyclopropyl)aniline (III-41)

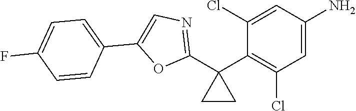 [SMILES: Nc1cc(Cl)c(C2(c3ncc(-c4ccc(F)cc4)o3)CC2)c(Cl)c1]

### Step 1: 2-amino-1-(4-fluorophenyl)ethan-1-one hydrochloride

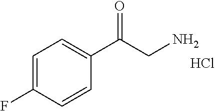 [SMILES: NCC(=O)c1ccc(F)cc1 ; Cl]

To a stirring solution of hexamethylenetetramine (1.696 g, 12.09 mmol) in chloroform (15 ml) was added 2-bromo-1-(4-fluorophenyl)ethanone (2.5 g, 11.52 mmol) and heated at 50° C. for 3 h. Product obtained was filtered and taken in 25 ml MeOH. To this was added 15 ml Conc. HCl and stirred for 16 h. Reaction mixture was filtered and MeOH layer was distilled out to get light yellow solid.

### Step 2: 1-(2,6-dichloro-4-nitrophenyl)-N-(2-(4-fluorophenyl)-2-oxoethyl)cyclopropane-1-carboxamide

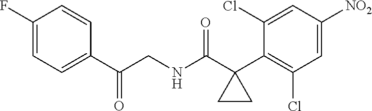 [SMILES: O=C(CNC(=O)C1(c2c(Cl)cc([N+](=O)[O-])cc2Cl)CC1)c1ccc(F)cc1]

A flask containing 1-(2,6-dichloro-4-nitrophenyl)cyclopropane-1-carboxylic acid (350 mg, 1.268 mmol) and thionyl chloride (1.388 ml, 19.02 mmol) was heated at 80° C. for 2 h. Excess thionyl chloride was distilled out to get crude acid chloride. In another flask, product of step 1 (481 mg, 2.54 mmol) was dissolved in THF (5 mL). To this was added TEA (0.707 ml, 5.07 mmol) and reaction mixture was cooled at 0° C. To this was added acid chloride prepared above dissolved in DCM (5 mL) and stirred at 0-10° C. for 2 h. Reaction mixture was diluted with DCM and washed with water. Organic layer was dried over Na₂SO₄ and distilled out. The crude product was column purified (15% EtOAc in Hexane) to get title compound. ¹H NMR (DMSO-d₆): 8.34 (s, 2H), 8.03-8.00 (m, 2H), 7.49 (t, 1H), 7.34 (t, 2H), 4.44 (d, J=5.6 Hz, 2H), 1.74-1.71 (m, 2H), 1.20-1.17 (m, 2H).

### Step 3: 2-(1-(2,6-dichloro-4-nitrophenyl)cyclopropyl)-5-(4-fluorophenyl)oxazole

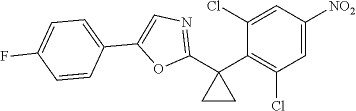 [SMILES: O=[N+]([O-])c1cc(Cl)c(C2(c3ncc(-c4ccc(F)cc4)o3)CC2)c(Cl)c1]

A solution of product of step 2 (330 mg, 0.803 mmol) and POCl₃ (3 ml) was refluxed for 16 h. Volatile mass was distilled out from reaction mixture and poured into ice water. Crude product was extracted with EtOAc and organic layer was distilled out to get crude product which was column purified (7% EtOAc in Hexane) to get title compound. 1H NMR (DMSO-d₆): 8.37 (s, 2H), 7.66-7.62 (m, 2H), 7.46 (s, 1H), 7.28 (t, 2H), 2.07-2.03 (m, 2H), 1.61-1.58 (m, 2H).

### Step 4: 3,5-dichloro-4-(1-(5-(4-fluorophenyl)oxazol-2-yl)cyclopropyl)aniline

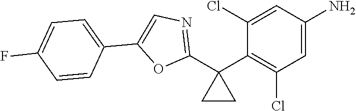 [SMILES: Nc1cc(Cl)c(C2(c3ncc(-c4ccc(F)cc4)o3)CC2)c(Cl)c1]

To a stirring solution of product of step 3 (220 mg, 0.560 mmol) in EtOAc (10 mL) was added SnCl₂.2H₂O (631 mg, 2.80 mmol) at RT and stirred for 12 h. Reaction mixture was diluted with EtOAc and basified with ammonia solution. organic layer was separated and washed with water. Organic layer was dried over Na₂SO₄ and distilled out to get title product as solid. 1H NMR (DMSO-d₆): 7.60-7.56 (m, 2H), 7.42 (s, 1H), 7.27 (t, 2H), 5.72 (s, 2H), 6.64 (s, 2H), 1.84-1.81 (m, 2H), 1.41-1.38 (m, 2H). ESI-MS (m/z): 363.00 (M+H)⁺.

Using appropriate starting materials and suitable modifications of the process described for the preparation of intermediate III-41, including suitable addition and/or deletion of steps as may be necessary, well within the scope of a person skilled in the art, the following intermediates (Table 3) were prepared in an analogues manner.

TABLE 3

| Intermediate | Structure | ESI-MS (m/z): (M + H)⁺ |
|---|---|---|
| III-42 | 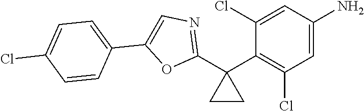 [SMILES: Nc1cc(Cl)c(C2(c3ncc(-c4ccc(Cl)cc4)o3)CC2)c(Cl)c1] | 378.90 |
| III-43 | 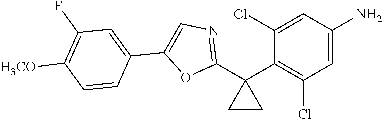 [SMILES: COc1ccc(-c2cnc(C3(c4c(Cl)cc(N)cc4Cl)CC3)o2)cc1F] | 393.00 |

### Preparation of 3,5-dichloro-4-(1-(5-(4-fluorophenyl)-1,3,4-oxadiazol-2-yl)cyclopropyl)aniline (III-44)

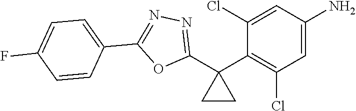 [SMILES: Nc1cc(Cl)c(C2(c3nnc(-c4ccc(F)cc4)o3)CC2)c(Cl)c1]

### Step 1: 2-(1-(2,6-dichloro-4-nitrophenyl)cyclopropyl)-5-(4-fluorophenyl)-1,3,4-oxadiazole

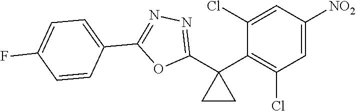 [SMILES: O=[N+]([O-])c1cc(Cl)c(C2(c3nnc(-c4ccc(F)cc4)o3)CC2)c(Cl)c1]

To a stirring solution of 1-(2,6-dichloro-4-nitrophenyl)cyclopropanecarboxylic acid (550 mg, 1.992 mmol) in POCl₃ (0.5 mL, 59.8 mmol) was added 4-fluorobenzohydrazide (307 mg, 1.992 mmol) at 0° C. Reaction mixture was refluxed for 16 h. Reaction mixture was poured in water and extracted with EtOAc. Organic layer was distilled out to get crude product which was column purified (5% EtOAc in Hexane) to get title product. ESI-MS (m/z): 394.00 (M+H)⁺.

### Step 2: 3,5-dichloro-4-(1-(5-(4-fluorophenyl)-1,3,4-oxadiazol-2-yl)cyclopropyl)aniline

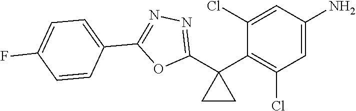 [SMILES: Nc1cc(Cl)c(C2(c3nnc(-c4ccc(F)cc4)o3)CC2)c(Cl)c1]

Prepared using product of step 1 by following similar procedure as described for step 6 in intermediate III-1. ¹H NMR (DMSO-d₆): 7.96 (m, 2H), 7.43-7.41 (m, 2H), 6.65 (s, 2H), 5.78 (s, 2H), 1.95-1.94 (m, 2H), 1.52-1.51 (m, 2H). ESI-MS (m/z): 364.00 (M+H)⁺.

### Preparation of intermediates of formula (IV) and (V)

### Preparation of 2-(4-(ethylsulfonyl)phenyl)acetic acid (IV-1)

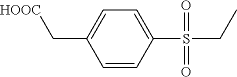

### Step 1: ethyl(phenyl)sulfane

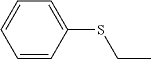

To a stirring solution of thiophenol (10 ml, 97 mmol) and K₂CO₃ (20.13 g, 146 mmol) in 100 ml acetone was added diethyl sulfate (13.96 ml, 107 mmol) at RT. Reaction mixture was stirred at RT for 12 h. Reaction mixture was poured in water and extracted with EtOAc. The organic layer was distilled out to get 10.2 g of title product as liquid. ¹H NMR (CDCl₃): 7.35-7.33 (m, 2H), 7.32-7.27 (m, 2H), 7.20-7.16 (m, 1H), 2.97 (q, J=7.4 Hz, 2H), 1.33 (t, J=7.2 Hz, 3H).

### Step 2: 1-(4-(ethylthio)phenyl)ethan-1-one

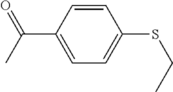

To a stirring solution of product of step 1 (50 g, 362 mmol) in DCM (500 ml) was added aluminum chloride (57.9 g, 434 mmol) at 0° C. To this was added acetyl chloride (30.9 ml, 434 mmol) at 0° C. and stirred for 3 h. The reaction mixture was slowly poured in to cold dil. HCl and product was extracted with DCM. The organic layer was distilled out to get 36.6 g of title product as liquid. ¹H NMR (CDCl₃): 7.86 (d, J=8.0 Hz, 2H), 7.30 (d, J=8.0 Hz, 2H), 3.05 (q, J=7.0 Hz, 2H), 2.58 (s, 3H), 1.39 (t, J=7.4 Hz, 3H).

### Step 3: 2-(4-(ethylthio)phenyl)acetic acid

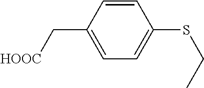

The mixture of product of step 2 (36.6 g, 203 mmol), morpholine (19.46 ml, 223 mmol) and sulfur (7.16 g, 223 mmol) was stirred at 130° C. for 7 h. Conc. HCl (50 ml) was added to above reaction mixture and refluxed continued for further 16 h. Reaction mixture was allowed to cooled to 25-30° C. and basified with aq. NaOH followed by extraction with EtOAc. Aqueous layer was acidified using dil. HCl to obtained solid material which was filtered and washed with water to get 26.3 g title product as solid. ¹H NMR (DMSO-d₆): 12.24 (bs, 1H), 7.24 (dd, J=2.0 & 6.4 Hz, 2H), 7.18 (d, J=8.4 Hz, 2H), 3.52 (s, 2H), 2.95 (q, J=7.4 Hz, 2H), 1.21 (t, J=7.2 Hz, 3H).

### Step 4: 2-(4-(ethylsulfonyl)phenyl)acetic acid

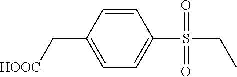

To a stirring solution of product of step 3 (12 g, 61.1 mmol) and vanadium pentoxide (100 mg) in CH₃CN (50 ml) was added 50% hydrogen peroxide (15 ml) at 0° C. and stirred for 2 h at RT. The reaction mixture was slowly poured to ice cold water and product obtained was extracted in EtOAc. The organic layer was distilled out to get 10 g of title product as solid. ¹H NMR (DMSO-d₆): 12.54 (bs, 1H), 7.83 (d, J=8.0 Hz, 2H), 7.55 (d, J=8.4 Hz, 2H), 3.73 (s, 2H), 3.30 (q, J=7.2 Hz, 2H), 1.09 (t, J=7.2 Hz, 3H).

### Preparation of sodium 2-(5-(ethylsulfonyl)pyridin-2-yl)acetate (IV-2)

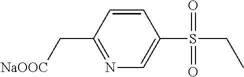

### Step 1: 2-chloro-5-(ethylthio)pyridine

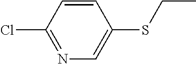

To a stirring solution of diethyl disulfide (16.91 ml, 137 mmol), tert-butyl nitrite (12.21 ml, 103 mmol) in DCE (25 ml) at 40° C. was added solution of 6-chloropyridin-3-amine (8.8 g, 68.5 mmol) in DCE (25 ml) over 1 h. Reaction mixture was stirred further 1 h at 40° C. and at RT for 3 h. Reaction mixture was diluted with water and organic layer was separated. Organic layer was washed with dil. HCl and then with water. The organic layer was distilled out to get crude product which was column purified (5% EtOAc in Hexane) to get title product. ¹H NMR (DMSO-d₆): 8.34 (d, J=2.4 Hz, 1H), 7.84 (dd, J=2.8 & 8.4 Hz, 1H), 7.47 (d, J=8.4 Hz, 1H), 3.06 (q, J=7.2 Hz, 2H), 1.22 (t, J=7.2 Hz, 3H).

### Step 2: 2-chloro-5-(ethylsulfonyl)pyridine

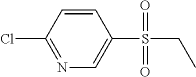

To a stirring solution of product of step 1 (6.5 g, 37.4 mmol), vanadium pentoxide (100 mg) in CH₃CN (65 ml) was added hydrogen peroxide (2.52 ml, 41.2 mmol) at −10° C. Reaction mixture was stirred further for 30 min. at 0° C. before it was diluted with water and EtOAc. Organic layer was separated and distilled out to get title product. ¹H NMR (DMSO-d₆): 8.88 (d, J=2.4 Hz, 1H), 8.33 (dd, J=2.8 & 8.4 Hz, 1H), 7.85 (d, J=8.4 Hz, 1H), 3.46 (q, J=7.2 Hz, 2H), 1.13 (t, J=7.6 Hz, 3H).

### Step 3: diethyl 2-(5-(ethylsulfonyl)pyridin-2-yl)malonate

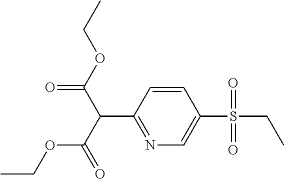 [SMILES: CCOC(=O)C(C(=O)OCC)c1ccc(S(=O)(=O)CC)cn1]

To a stirring suspension of product of step 2 (6.6 g, 32.1 mmol) and Cs₂CO₃ (11.50 g, 35.3 mmol) in DMSO (30 ml) was added diethyl malonate (5.38 ml, 35.3 mmol) at RT. Reaction mixture was stirred at 90° C. for 4 h. Reaction mixture was poured in water and extracted with EtOAc. Organic layer and washed with water and distilled out to get crude product which was column purified (20% EtOAc in Hexane) to get title product. ¹H NMR (CDCl₃): 9.05 (d, J=2.0 Hz, 1H), 8.23 (dd, J=2.0 & 8.0 Hz, 1H), 7.77 (d, J=8.0 Hz, 1H), 5.05 (s, 1H), 4.34-4.22 (m, 4H), 3.20-3.10 (m, 2H), 1.36-1.25 (m, 9H).

### Step 4: sodium 2-(5-(ethylsulfonyl)pyridin-2-yl)acetate

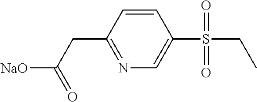

To a stirring solution of product of step 3 (7.15 g, 21.71 mmol) in MeOH (30 ml) was added solution of NaOH (2.60 g, 65.1 mmol) in water (10 ml) and stirred for 3 h at 25-30° C. The solvent mixture was evaporated completely using freeze drier to get title product which was used directly in the next step. ¹H NMR (DMSO-d₆): 8.79 (d, J=2.4 Hz, 1H), 8.09 (dd, J=2.4 & 8.4 Hz, 1H), 7.57 (d, J=8.4 Hz, 1H), 3.51 (s, 2H), 3.31 (q 2H), 1.11 (t, J=7.2 Hz, 3H).

### Preparation of 2-(4-(N-((benzyloxy)carbonyl)ethylsulfonimidoyl)phenyl)acetic acid (IV-3)

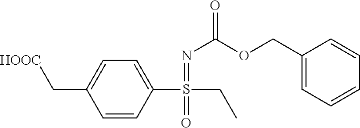 [SMILES: CCS(=O)(=NC(=O)OCc1ccccc1)c1ccc(CC(=O)O)cc1]

### Step 1: ethyl 2-(4-(ethylthio)phenyl)acetate

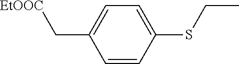

To a stirring suspension of 2-(4-(ethylthio)phenyl)acetic acid (5.0 g, 25.5 mmol) and NaHCO₃ (3.21 g, 38.2 mmol) in 50 ml acetone was added diethyl sulfate (3.66 ml, 28.0 mmol) at RT. Reaction mixture was refluxed for 12 h. Reaction mixture was poured in water and product was extracted with EtOAc. Organic layer was distilled out to get title product. ¹H NMR (CDCl₃): 7.32-7.26 (m, 2H), 7.22-7.19 (m, 2H), 4.17 (q, J=6.8 Hz, 2H), 1.34-1.23 (m, 3H+3H).

### Step 2: ethyl 2-(4-(ethylsulfinyl)phenyl)acetate

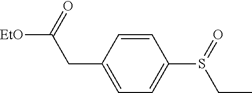 [SMILES: CCOC(=O)Cc1ccc(S(=O)CC)cc1]

To a stirring solution of step 1 (5.0 g, 22.29 mmol) and vanadium pentoxide (50 mg) in CH₃CN (50 ml) was added 50% hydrogen peroxide (2.504 ml, 24.52 mmol) at 0° C. Reaction mixture was stirred for 2 h before it was poured in to ice cold water and extracted with EtOAc. Organic layer was distilled out to get title compound. ¹H NMR (CDCl₃): 7.59-7.57 (m, 2H), 7.46 (d, 2H), 4.19 (q, J=7.2 Hz, 2H), 3.68 (s, 2H), 2.95-2.86 (m, 1H), 2.82-2.73 (m, 1H), 1.31-1.19 (m, 3H+3H).

### Step 3: ethyl 2-(4-(ethylsulfonimidoyl)phenyl)acetate

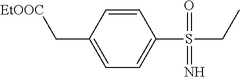 [SMILES: CCOC(=O)Cc1ccc(S(=N)(=O)CC)cc1]

To a stirring solution of product of step 2 (4.84 g, 20.14 mmol) in chloroform (20 ml) was added sodium azide (5.24 g, 81 mmol) at 0° C. followed by slow addition of sulfuric acid (8.59 ml, 161 mmol). Reaction mixture was stirred at RT for 16 h. Chloroform was removed form reaction mixture and residue obtained was basified by K₂CO₃ solution followed by extraction with DCM. Organic solvent was dried over Na₂SO₄ and distilled out to get 1.2 g of title product. ¹H NMR (DMSO-d₆): 7.84-7.80 (m, 2H), 7.51-7.43 (m, 2H), 4.2 (bs, 1H), 4.11 (q, 2H), 3.75 (s, 2H), 3.12-3.06 (m, 2H), 1.18 (t, 3H), 1.08 (t, 3H).

### Step 4: ethyl 2-(4-(N-((benzyloxy)carbonyl)ethylsulfonimidoyl)phenyl)acetate

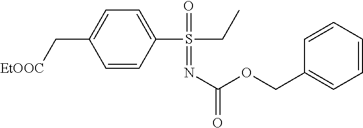 [SMILES: CCOC(=O)Cc1ccc(S(=O)(CC)=NC(=O)OCc2ccccc2)cc1]

To a stirring solution of product of step 3 (1.2 g, 4.70 mmol) and pyridine (0.570 ml, 7.05 mmol) in DCM (10 mL) was added benzyl chloroformate (1.534 ml, 5.17 mmol) at 0-10° C. and stirred at this temp for 1 h. Reaction mixture was diluted with DCM and washed with dil. HCl. The crude product was column purified (35% EtOAc in Hexane) to get 700 mg of title product. ESI-MS (m/z): 390.35 (M+H)⁺.

### Step 5: 2-(4-(N-((benzyloxy)carbonyl)ethylsulfonimidoyl) phenyl)acetic acid

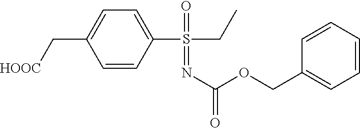 [SMILES: CCS(=O)(=NC(=O)OCc1ccccc1)c1ccc(CC(=O)O)cc1]

To a stirring solution of product of step 4 (700 mg, 1.797 mmol) in water (5 ml) and THF (5 ml) was added LiOH.H₂O (108 mg, 4.49 mmol) and stirred for 5 h. Reaction mixture was diluted with water, acidified by dil. HCl and extracted with EtOAc. Organic layer was washed with water and dried over Na₂SO₄ and distilled out to get 500 mg of title product. ¹H NMR (DMSO-d₆): 12.5 (bs, 1H), 7.83 (d, J=8.4 Hz, 2H), 7.57 (d, J=8.4 Hz, 2H), 7.34-7.28 (m, 3H), 7.23-7.21 (m, 2H), 4.98 (q, 2H), 3.75 (s, 2H), 3.58 (q, 2H), 1.08 (t, J=7.2 Hz, 3H).

### Preparation of 2-(4-(N-cyclopropylsulfamoyl)phenyl)acetic acid (IV-4)

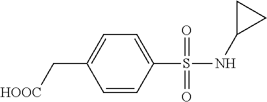 [SMILES: O=C(O)Cc1ccc(S(=O)(=O)NC2CC2)cc1]

### Step 1: 2-(4-(chlorosulfonyl)phenyl)acetic acid

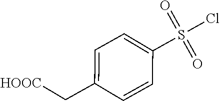 [SMILES: O=C(O)Cc1ccc(S(=O)(=O)Cl)cc1]

A mixture of phenyl acetic acid (25 g, 184 mmol) and chlorosulfonic acid (123 ml, 1836 mmol) was cooled to 0° C. The reaction mixture was stirred at 0° C. for 1 h and then at RT for 4 h. Reaction mixture was poured in ice water. Precipitated solid was filtered and washed with water. Solid obtained was dried and purified in chloroform to get title product.

### Step 2: 2-(4-(N-cyclopropylsulfamoyl)phenyl)acetic acid

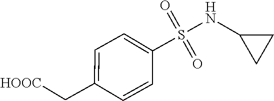 [SMILES: O=C(O)Cc1ccc(S(=O)(=O)NC2CC2)cc1]

To a stirring solution of cyclopropanamine (0.973 g, 17.05 mmol) in MeOH (10 ml) cooled at 0° C. was added product of step 1 (2.0 g, 8.52 mmol). Reaction mixture was stirred at RT for 1 h. MeOH from reaction mixture was evaporated under reduced pressure followed by dilution with water (10 ml). Reaction mixture was cooled and acidified with dil. HCl to get solid product which was extracted in EtOAc (20 ml). Organic layer was dried over Na₂SO₄ and distilled out to get title product. ¹H NMR (DMSO-d₆): 12.49 (bs, 1H), 7.88 (d, 1H), 7.75 (d, J=8.4 Hz, 2H), 7.49 (d, J=8.0 Hz, 2H), 3.70 (s, 2H), 2.10-2.04 (m, 1H), 0.61-0.59 (m, 2H), 0.47-0.44 (m, 2H).

### Preparation of 2-(6-(ethylthio)pyridin-3-yl)acetic acid (V-1)

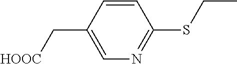

### Step 1: 1-(6-(ethylthio)pyridin-3-yl)ethan-1-one

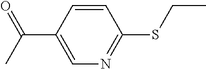

To a stirring suspension of NaH (116 mg, 2.89 mmol) in DMF (2 ml) was added ethane thiol (0.214 ml, 2.89 mmol) followed by 1-(6-chloropyridin-3-yl)ethanone (300 mg, 1.928 mmol) at 0° C. and stirred for 3 h at RT. Reaction mixture was poured in to ice water and crude product was extracted with EtOAc, which was further purified by column chromatography to get 160 mg of title product. ¹H NMR (CDCl₃): 8.98 (d, J=2.4 Hz, 1H), 8.02 (dd, J=2.4 & 8.4 Hz, 1H), 7.25 (dd, J=0.8 & 8.4 Hz, 1H), 3.27 (q, J=7.2 Hz, 2H), 2.59 (s, 3H), 1.41 (t, J=7.2 Hz, 3H).

### Step 2: 2-(6-(ethylthio)pyridin-3-yl)acetic acid

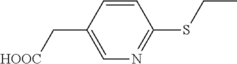

The mixture of product obtained from step 1 (2.5 g, 13.79 mmol), morpholine (1.32 ml, 15.17 mmol) and sulfur (0.486 g, 15.17 mmol) was stirred at 130° C. for 7 h. Then Conc. HCl (30 ml) was added to above reaction mixture and refluxed for 16 h. Reaction mixture was allowed to cooled at RT. Basified by aq. NaOH and washed with EtOAc. Aqueous layer was acidified and solid obtained was filtered and washed with water to get 1.5 g of title product as solid. ¹H NMR (DMSO-d₆): 12.5 (bs, 1H), 8.30 (d, J=1.6 Hz, 1H), 7.54 (dd, J=2.4 & 8.0 Hz, 1H), 7.23 (d, J=8.4 Hz, 1H), 3.56 (s, 2H), 3.11 (q, 2H), 1.27 (t, 3H).

### Preparation of sodium 2-(5-(ethylthio)pyrazin-2-yl)acetate (V-2)

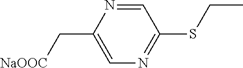

### Step 1: 1-(tert-butyl) 3-ethyl 2-(5-bromopyrazin-2-yl)malonate

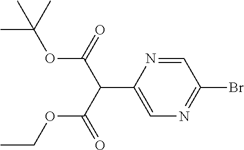 [SMILES: CCOC(=O)C(C(=O)OC(C)(C)C)c1cnc(Br)cn1]

To stirring solution of tert-butyl ethyl malonate (0.879 g, 5.04 mmol) in DMF (5 ml) was added NaH (0.131 g, 5.46 mmol) at 0° C. The reaction mixture was stirred at 0° C. for 30 min. To this was added 2,5-dibromopyrazine (1 g, 4.20 mmol) portion wise at 0° C. and reaction mixture was stirred at RT for 2 h. After complete conversion of starting material, the reaction mixture was diluted with 20 ml saturated solution of NH₄Cl and product was extracted with EtOAc (25 ml). Organic layer was washed with brine, dried over Na₂SO₄, and filtered. Removal of the solvent under reduced pressure gave product as brown oil which was further purified by column chromatography (20% EtOAc in Hexane) to get yellow oil as pure product. ESI-MS (m/z): 347. (M+H)⁺.

### Step 2: ethyl 2-(5-bromopyrazin-2-yl)acetate

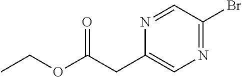

To a stirring solution of product of step 1 (0.5 g, 1.448 mmol) in DCM (5 ml) was added TFA (2.9 mL, 37.7 mmol) at 0° C. and stirred for 1 h at 0-5° C. After complete conversion of starting material, the reaction mixture was diluted with DCM (20 ml) and washed with water. Organic layer was evaporated under reduced pressure to get the crude product as yellow oil, which was taken in to the next step without further purification. ESI-MS (m/z): 246.95 (M+H)⁺.

### Step 3: ethyl 2-(5-(ethylthio)pyrazin-2-yl)acetate

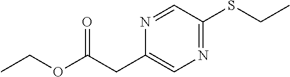

To a stirring solution of product of step 2 (0.300 g, 1.224 mmol) in DMF (3 ml) was added NaH (0.054 g, 1.347 mmol) at 0° C. To this was added ethanethiol (0.11 mL, 1.469 mmol) at 0° C. and reaction mixture was stirred at RT for 1 h. After complete conversion of starting material, the reaction mixture was diluted with 10 ml saturated solution of NH₄Cl and product was extracted with EtOAc (15 ml). Organic layer was washed with brine, dried over Na₂SO₄, and filtered. Removal of the solvent under reduced pressure gave product as brown oil which was further purified by column chromatography (5% EtOAc in Hexane) to get yellow oil as pure product. ESI-MS (m/z): 226.65 (M+H)⁺.

### Step 4: sodium 2-(5-(ethylthio)pyrazin-2-yl)acetate

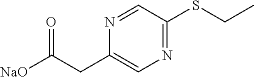

To a stirring solution of product of step 3 (0.210 g, 0.928 mmol) in THF (2 ml) and water (1 ml) was added NaOH (0.093 g, 2.320 mmol) at 25° C. The reaction mixture was stirred at 25° C. for 1 h. After complete conversion of starting material, THF was removed from reaction mixture and then diluted with 2 ml water and then reaction mixture was lyophilized to get yellow solid as a title product. ¹H NMR (D₂O): 8.46 (d, J=1.2 Hz, 1H), 8.37 (d, J=1.6 Hz, 1H), 3.72 (s, 2H), 3.21 (q, J=7.6 Hz, 2H), 1.37 (t, J=7.2 Hz, 3H). ESI-MS (m/z): 198.55 (M+H)⁺.

### Preparation of compounds of formula (I)

### Example 1

### Preparation of N-(3,5-dichloro-4-(1-(5-(4-fluorophenyl)-1,2,4-oxadiazol-3-yl)cyclopropyl)phenyl)-2-(4-(ethylsulfonyl)phenyl)acetamide

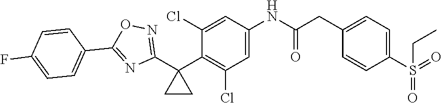 [SMILES: CCS(=O)(=O)c1ccc(CC(=O)Nc2cc(Cl)c(C3(c4noc(-c5ccc(F)cc5)n4)CC3)c(Cl)c2)cc1]

To a stirring solution of intermediate IV-1 (94 mg, 0.412 mmol) and HOBT (63 mg, 0.412 mmol) in DCM (5 ml) was added intermediate III-1 (150 mg, 0.412 mmol) followed by DIPEA (0.144 ml, 0.824 mmol) and EDC.HCl (118 mg, 0.618 mmol). Reaction mixture was stirred for 16 h at RT. Reaction mixture was diluted with DCM and water. DCM layer was separated and distilled out to get crude product which was purified by preparative HPLC to get title product. ¹H NMR (DMSO-d₆): 10.61 (s, 1H), 8.12-8.08 (m, 2H), 7.86 (d, J=8.4 Hz, 2H), 7.75 (s, 2H), 7.61 (d, J=8.4 Hz, 2H), 7.44 (t, 2H), 3.84 (s, 2H), 3.30 (q, J=7.6 Hz, 2H), 1.91-1.88 (m, 2H), 1.54-1.51 (m, 2H), 1.09 (t, J=7.2 Hz, 3H). ESI-MS (m/z): 574.80 (M+H)⁺.

Using appropriate starting materials and suitable modifications of the process described in example 1, including suitable addition and/or deletion of steps as may be necessary, well within the scope of a person skilled in the art, the following compounds were prepared in an analogues manner.

### Example 2

### Preparation of N-(3,5-dichloro-4-(1-(5-phenyl-1,2,4-oxadiazol-3-yl)cyclopropyl) phenyl)-2-(4-(ethylsulfonyl)phenyl)acetamide

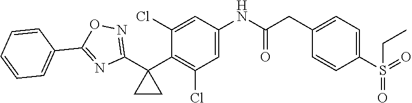 [SMILES: CCS(=O)(=O)c1ccc(CC(=O)Nc2cc(Cl)c(C3(c4noc(-c5ccccc5)n4)CC3)c(Cl)c2)cc1]

Prepared using intermediate IV-1 and intermediate III-2. ¹H NMR (DMSO-d₆): 10.60 (s, 1H), 8.04 (d, J=7.6 Hz, 2H), 7.86 (d, J=8.0 Hz, 2H), 7.75 (s, 2H), 7.69-7.59 (m, 5H), 3.84 (s, 2H), 3.30 (q, J=7.2 Hz, 2H), 1.90 (bd, 2H), 1.54 (bd, 2H), 1.10 (t, J=7.2 Hz, 3H). ESI-MS (m/z): 556.55 (M+H)⁺.

### Example 3

### Preparation of N-(3,5-dichloro-4-(1-(5-(4-(trifluoromethyl)phenyl)-1,2,4-oxadiazol-3-yl)cyclopropyl)phenyl)-2-(4-(ethylsulfonyl)phenyl)acetamide

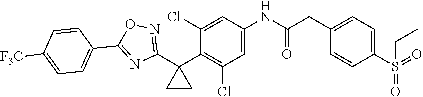 [SMILES: CCS(=O)(=O)c1ccc(CC(=O)Nc2cc(Cl)c(C3(c4noc(-c5ccc(C(F)(F)F)cc5)n4)CC3)c(Cl)c2)cc1]

Prepared using intermediate IV-1 and intermediate III-3. ¹H NMR (DMSO-d₆): 10.63 (s, 1H), 8.26 (d, J=8.0 Hz, 2H), 7.99 (d, J=8.4 Hz, 2H), 7.86 (d, J=8.4 Hz, 2H), 7.75 (s, 2H), 7.61 (d, J=8.4 Hz, 2H), 3.84 (s, 2H), 3.30 (q, J=7.2 Hz, 2H), 1.93-1.90 (m, 2H), 1.58-1.55 (m, 2H), 1.09 (t, J=7.2 Hz, 3H). ESI-MS (m/z): 624.05 (M+H)⁺.

### Example 4

### Preparation of N-(3,5-dichloro-4-(1-(5-(p-tolyl)-1,2,4-oxadiazol-3-yl)cyclopropyl) phenyl)-2-(4-(ethylsulfonyl)phenyl)acetamide

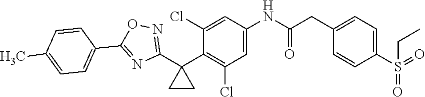 [SMILES: CCS(=O)(=O)c1ccc(CC(=O)Nc2cc(Cl)c(C3(c4noc(-c5ccc(C)cc5)n4)CC3)c(Cl)c2)cc1]

Prepared using intermediate IV-1 and intermediate III-4. ¹H NMR (DMSO-d₆): 10.62 (s, 1H), 7.93 (d, J=8.4 Hz, 2H), 7.86 (d, J=8.4 Hz, 2H), 7.74 (s, 2H), 7.61 (d, J=8.4 Hz, 2H), 7.42 (d, J=8.0 Hz, 2H), 3.84 (s, 2H), 3.30 (q, J=7.2 Hz, 2H), 2.40 (s, 3H), 1.90-1.87 (m, 2H), 1.53-1.50 (m, 2H), 1.09 (t, J=7.2 Hz, 3H). ESI-MS (m/z): 568.05 (M−H).

### Example 5

### Preparation of N-(3,5-dichloro-4-(1-(5-(3,4-difluorophenyl)-1,2,4-oxadiazol-3-yl)cyclopropyl)phenyl)-2-(4-(ethylsulfonyl)phenyl)acetamide

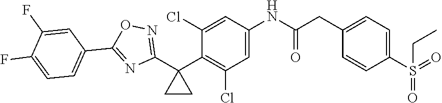 [SMILES: CCS(=O)(=O)c1ccc(CC(=O)Nc2cc(Cl)c(C3(c4noc(-c5ccc(F)c(F)c5)n4)CC3)c(Cl)c2)cc1]

Prepared using intermediate IV-1 and intermediate III-5. ¹H NMR (DMSO-d₆): 10.62 (s, 1H), 8.10 (m, 1H), 7.92 (m, 1H), 7.86 (d, J=8.4 Hz, 2H), 7.75 (s, 2H), 7.70-7.68 (m, 1H), 7.61 (d, J=8.4 Hz, 2H), 3.84 (s, 2H), 3.30 (q, J=7.2 Hz, 2H), 1.91-1.88 (m, 2H), 1.55-1.52 (m, 2H), 1.09 (t, J=7.2 Hz, 3H). ESI-MS (m/z): 592.00 (M+H)⁺.

### Example 6

### Preparation of N-(3,5-dichloro-4-(1-(5-(3-fluorophenyl)-1,2,4-oxadiazol-3-yl)cyclopropyl)phenyl)-2-(4-(ethylsulfonyl)phenyl)acetamide

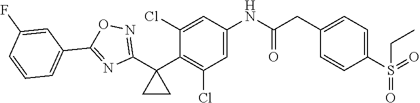 [SMILES: CCS(=O)(=O)c1ccc(CC(=O)Nc2cc(Cl)c(C3(c4noc(-c5cccc(F)c5)n4)CC3)c(Cl)c2)cc1]

Prepared using intermediate IV-1 and intermediate III-6. ¹H NMR (DMSO-d₆): 10.63 (s, 1H), 7.90-7.81 (m, 4H), 7.75 (s, 2H), 7.67-7.57 (m, 4H), 3.84 (s, 2H), 3.28 (q, J=7.2 Hz, 2H), 1.90 (bd, 2H), 1.56 (bd, 2H), 1.09 (t, J=7.2 Hz, 3H). ESI-MS (m/z): 574.05 (M+H)⁺.

### Example 7

### Preparation of N-(3,5-dichloro-4-(1-(5-(2,4-difluorophenyl)-1,2,4-oxadiazol-3-yl)cyclopropyl)phenyl)-2-(4-(ethylsulfonyl)phenyl)acetamide

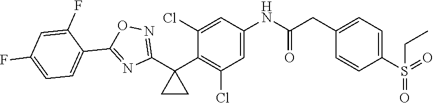 [SMILES: CCS(=O)(=O)c1ccc(CC(=O)Nc2cc(Cl)c(C3(c4noc(-c5ccc(F)cc5F)n4)CC3)c(Cl)c2)cc1]

Prepared using intermediate IV-1 and intermediate III-7. ¹H NMR (DMSO-d₆): 10.63 (s, 1H), 8.15 (m, 1H), 7.86 (d, J=8.4 Hz, 2H), 7.75 (s, 2H), 7.61-7.59 (m, 3H), 7.34 (m, 1H), 3.84 (s, 2H), 3.30 (q, J=7.6 Hz, 2H), 1.89-1.87 (m, 2H), 1.56-1.54 (m, 2H), 1.09 (t, J=7.6 Hz, 3H). ESI-MS (m/z): 592.00 (M+H)⁺.

### Example 8

### Preparation of N-(3,5-dichloro-4-(1-(5-(2,4,6-trifluorophenyl)-1,2,4-oxadiazol-3-yl)cyclopropyl)phenyl)-2-(4-(ethylsulfonyl)phenyl)acetamide

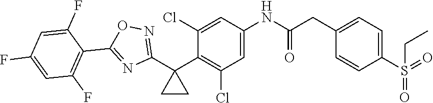 [SMILES: CCS(=O)(=O)c1ccc(CC(=O)Nc2cc(Cl)c(C3(c4noc(-c5c(F)cc(F)cc5F)n4)CC3)c(Cl)c2)cc1]

Prepared using intermediate IV-1 and intermediate III-8. ¹H NMR (DMSO-d₆): 10.62 (s, 1H), 7.86 (d, J=8.4 Hz, 2H), 7.75 (s, 2H), 7.61 (d, J=8.0 Hz, 2H), 7.54 (m, 2H), 3.84 (s, 2H), 3.30 (q, J=7.6 Hz, 2H), 1.87 (bd, 2H), 1.56 (bd, 2H), 1.09 (t, J=7.2 Hz, 3H). ESI-MS (m/z): 610.05 (M+H)⁺.

### Example 9

### Preparation of N-(3,5-dichloro-4-(1-(5-(2-chloro-4-fluorophenyl)-1,2,4-oxadiazol-3-yl)cyclopropyl)phenyl)-2-(4-(ethylsulfonyl)phenyl)acetamide

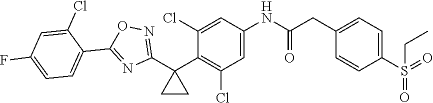 [SMILES: CCS(=O)(=O)c1ccc(CC(=O)Nc2cc(Cl)c(C3(c4noc(-c5ccc(F)cc5Cl)n4)CC3)c(Cl)c2)cc1]

Prepared using intermediate IV-1 and intermediate III-9. ¹H NMR (DMSO-d₆): 10.61 (s, 1H), 8.12 (m, 1H), 7.86 (d, J=8.4 Hz, 2H), 7.78 (d, J=2.8 Hz, 1H), 7.75 (s, 2H), 7.61 (d, J=8.4 Hz, 2H), 7.5-7.4 (m, 1H), 3.84 (s, 2H), 3.32 (q, J=7.2 Hz, 2H), 1.89 (bd, 2H), 1.55 (bd, 2H), 1.09 (t, J=7.6 Hz, 3H). ESI-MS (m/z): 608.50 (M+H)⁺.

### Example 10

### Preparation of N-(3,5-dichloro-4-(1-(5-(3-chloro-4-fluorophenyl)-1,2,4-oxadiazol-3-yl)cyclopropyl)phenyl)-2-(4-(ethylsulfonyl)phenyl)acetamide

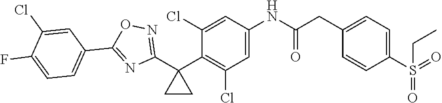 [SMILES: CCS(=O)(=O)c1ccc(CC(=O)Nc2cc(Cl)c(C3(c4noc(-c5ccc(F)c(Cl)c5)n4)CC3)c(Cl)c2)cc1]

Prepared using intermediate IV-1 and intermediate III-10. ¹H NMR (DMSO-d₆): 10.61 (s, 1H), 8.23 (dd, J=2.4 & 7.2 Hz, 1H), 8.1 (m, 1H), 7.86 (dd, J=2.0 & 6.4 Hz, 2H), 7.75 (s, 2H), 7.66 (t, 1H), 7.61 (d, J=8.4 Hz, 2H), 3.84 (s, 2H), 3.31 (q, J=7.2 Hz, 2H), 1.90 (bd, 2H), 1.54 (bd, 2H), 1.09 (t, J=7.2 Hz, 3H). ESI-MS (m/z): 609 (M+H)⁺.

### Example 11

### Preparation of N-(3,5-dichloro-4-(1-(5-(3,4-dichlorophenyl)-1,2,4-oxadiazol-3-yl)cyclopropyl)phenyl)-2-(4-(ethylsulfonyl)phenyl)acetamide

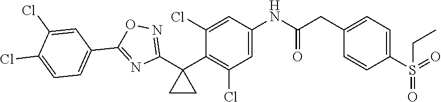 [SMILES: CCS(=O)(=O)c1ccc(CC(=O)Nc2cc(Cl)c(C3(c4noc(-c5ccc(Cl)c(Cl)c5)n4)CC3)c(Cl)c2)cc1]

Prepared using intermediate IV-1 and intermediate III-11. ¹H NMR (DMSO-d₆): 10.61 (s, 1H), 8.22 (d, J=2.0 Hz, 1H), 8.01 (dd, J=2.0 & 8.4 Hz, 1H), 7.89 (d, J=8.4 Hz, 1H), 7.86 (d, J=8.4 Hz, 2H), 7.75 (s, 2H), 7.61 (d, J=8.4 Hz, 2H), 3.84 (s, 2H), 3.28 (q, J=7.6 Hz, 2H), 1.92 (m, 2H), 1.56 (m, 2H), 1.09 (t, J=7.2 Hz, 3H). ESI-MS (m/z): 626.00 (M+H)⁺.

### Example 12

### Preparation of N-(3,5-dichloro-4-(1-(5-(2,4-dichlorophenyl)-1,2,4-oxadiazol-3-yl)cyclopropyl)phenyl)-2-(4-(ethylsulfonyl)phenyl)acetamide

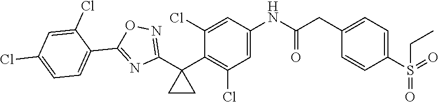 [SMILES: CCS(=O)(=O)c1ccc(CC(=O)Nc2cc(Cl)c(C3(c4noc(-c5ccc(Cl)cc5Cl)n4)CC3)c(Cl)c2)cc1]

Prepared using intermediate IV-1 and intermediate III-12. ¹H NMR (DMSO-d₆): 10.61 (s, 1H), 8.08 (d, J=8.8 Hz, 1H), 7.93 (d, J=2.0 Hz, 1H), 7.86 (d, J=8.4 Hz, 2H), 7.75 (s, 2H), 7.67 (dd, J=2.0 & 8.4 Hz, 1H), 7.61 (d, J=8.4 Hz, 2H), 3.84 (s, 2H), 3.30 (q, J=7.6 Hz, 2H), 1.90 (bd, 2H), 1.56 (bd, 2H), 1.09 (t, J=7.6 Hz, 3H). ESI-MS (m/z): 626.00 (M+H)⁺.

### Example 13

### Preparation of N-(3,5-dichloro-4-(1-(5-(4-chlorophenyl)-1,2,4-oxadiazol-3-yl) cyclopropyl)phenyl)-2-(4-(ethylsulfonyl)phenyl)acetamide

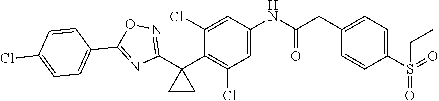 [SMILES: CCS(=O)(=O)c1ccc(CC(=O)Nc2cc(Cl)c(C3(c4noc(-c5ccc(Cl)cc5)n4)CC3)c(Cl)c2)cc1]

Prepared using intermediate IV-1 and intermediate III-13. ¹H NMR (DMSO-d₆): 10.61 (s, 1H), 8.05 (d, J=8.4 Hz, 2H), 7.86 (d, J=8.4 Hz, 2H), 7.75 (s, 2H), 7.69 (d, J=8.4 Hz, 2H), 7.61 (d, J=8.4 Hz, 2H), 3.84 (s, 2H), 3.31 (q, J=7.6 Hz, 2H), 1.90 (bd, 2H), 1.54 (bd, 2H), 1.09 (t, J=7.2 Hz, 3H). ESI-MS (m/z): 592.00 (M+H)⁺.

### Example 14

### Preparation of N-(3,5-dichloro-4-(1-(5-(4-cyanophenyl)-1,2,4-oxadiazol-3-yl) cyclopropyl)phenyl)-2-(4-(ethylsulfonyl)phenyl)acetamide

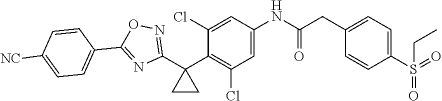 [SMILES: CCS(=O)(=O)c1ccc(CC(=O)Nc2cc(Cl)c(C3(c4noc(-c5ccc(C#N)cc5)n4)CC3)c(Cl)c2)cc1]

Prepared using intermediate IV-1 and intermediate III-16. ¹H NMR (DMSO-d₆): 10.63 (s, 1H), 8.21 (d, J=8.4 Hz, 2H), 8.08 (d, J=8.4 Hz, 2H), 7.86 (d, J=8.4 Hz, 2H), 7.75 (s, 2H), 7.61 (d, J=8.4 Hz, 2H), 3.84 (s, 2H), 3.30 (q, J=7.2 Hz, 2H), 1.91 (bd, 2H), 1.56 (bd, 2H), 1.09 (t, J=7.2 Hz, 3H). ESI-MS (m/z): 581.05 (M+H)⁺.

### Example 15

### Preparation of N-(3,5-dichloro-4-(1-(5-(2-fluoro-4-methoxyphenyl)-1,2,4-oxadiazol-3-yl)cyclopropyl)phenyl)-2-(4-(ethylsulfonyl)phenyl)acetamide

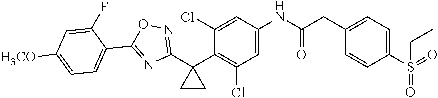 [SMILES: CCS(=O)(=O)c1ccc(CC(=O)Nc2cc(Cl)c(C3(c4noc(-c5ccc(OC)cc5F)n4)CC3)c(Cl)c2)cc1]

Prepared using intermediate IV-1 and intermediate III-17. ¹H NMR (DMSO-d₆): 10.62 (s, 1H), 7.97 (t, 1H), 7.86 (d, J=8.4 Hz, 2H), 7.74 (s, 2H), 7.61 (d, J=8.4 Hz, 2H), 7.12 (dd, J=2.4 & 8.8 Hz, 1H), 7.00 (dd, J=2.4 & 9.2 Hz, 1H), 3.87 (s, 3H), 3.84 (s, 2H), 3.30 (q, J=7.6 Hz, 2H), 1.90-1.86 (m, 2H), 1.53-1.50 (m, 2H), 1.09 (t, J=7.2 Hz, 3H). ESI-MS (m/z): 604.05 (M+H)⁺.

### Example 16

### Preparation of N-(3,5-dichloro-4-(1-(5-(thiophen-2-yl)-1,2,4-oxadiazol-3-yl) cyclopropyl)phenyl)-2-(4-(ethylsulfonyl)phenyl)acetamide

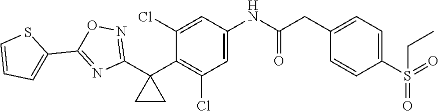 [SMILES: CCS(=O)(=O)c1ccc(CC(=O)Nc2cc(Cl)c(C3(c4noc(-c5cccs5)n4)CC3)c(Cl)c2)cc1]

Prepared using intermediate IV-1 and intermediate III-18. ¹H NMR (DMSO-d₆): 10.62 (s, 1H), 8.07-8.05 (m, 1H), 7.96-7.94 (m, 1H), 7.86 (d, J=8 Hz, 2H), 7.74 (s, 2H), 7.61 (d, J=8.4 Hz, 2H), 7.32-7.30 (m, 1H), 3.83 (s, 2H), 3.30 (q, J=7.6 Hz, 2H), 1.87-1.84 (m, 2H), 1.53-1.50 (m, 2H), 1.09 (t, J=7.2 Hz, 3H). ESI-MS (m/z): 562.5 (M+H)⁺.

### Example 17

### Preparation of N-(3,5-dichloro-4-(1-(5-(5-chlorothiophen-2-yl)-1,2,4-oxadiazol-3-yl)cyclopropyl)phenyl)-2-(4-(ethylsulfonyl)phenyl)acetamide

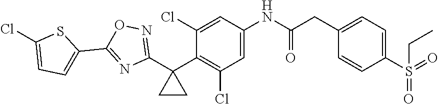 [SMILES: CCS(=O)(=O)c1ccc(CC(=O)Nc2cc(Cl)c(C3(c4noc(-c5ccc(Cl)s5)n4)CC3)c(Cl)c2)cc1]

Prepared using intermediate IV-1 and intermediate III-19. ¹H NMR (DMSO-d₆): 10.61 (s, 1H), 7.86-7.83 (m, 3H), 7.74 (s, 2H), 7.53 (d, J=8.4 Hz, 2H), 7.38 (d, J=4.4 Hz, 1H), 3.83 (s, 2H), 3.28 (q, J=7.6 Hz, 2H), 1.86-1.83 (m, 2H), 1.53-1.50 (m, 2H), 1.09 (t, J=7.6 Hz, 3H). ESI-MS (m/z): 598.00 (M+H)⁺.

### Example 18

### Preparation of N-(3,5-dichloro-4-(1-(5-(5-methylthiophen-2-yl)-1,2,4-oxadiazol-3-yl)cyclopropyl)phenyl)-2-(4-(ethylsulfonyl)phenyl)acetamide

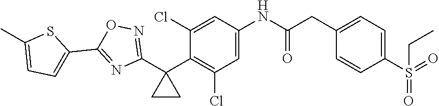 [SMILES: CCS(=O)(=O)c1ccc(CC(=O)Nc2cc(Cl)c(C3(c4noc(-c5ccc(C)s5)n4)CC3)c(Cl)c2)cc1]

Prepared using intermediate IV-1 and intermediate III-20. ¹H NMR (DMSO-d₆): 10.61 (s, 1H), 7.85 (d, J=8.4 Hz, 2H), 7.76 (d, J=3.6 Hz, 1H), 7.73 (s, 2H), 7.61 (d, J=8.0 Hz, 2H), 7.03 (d, J=3.6 Hz, 1H), 3.83 (s, 2H), 3.28 (q, J=7.2 Hz, 2H), 2.54 (s, 3H), 1.85-1.82 (m, 2H), 1.51-1.48 (m, 2H), 1.09 (t, J=7.2 Hz, 3H). ESI-MS (m/z): 576.00 (M+H)⁺.

### Example 19

### Preparation of N-(3,5-dichloro-4-(1-(5-(pyridin-3-yl)-1,2,4-oxadiazol-3-yl)cyclopropyl)phenyl)-2-(4-(ethylsulfonyl)phenyl)acetamide

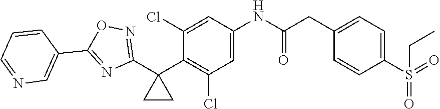 [SMILES: CCS(=O)(=O)c1ccc(CC(=O)Nc2cc(Cl)c(C3(c4noc(-c5cccnc5)n4)CC3)c(Cl)c2)cc1]

Prepared using intermediate IV-1 and intermediate III-21. ¹H NMR (DMSO-d₆): 10.62 (s, 1H), 9.19-9.18 (m, 1H), 8.85-8.84 (m, 1H), 8.42-8.39 (m, 1H), 7.86 (d, J=8.4 Hz, 2H), 7.75 (s, 2H), 7.65-7.59 (m, 3H), 3.84 (s, 2H), 3.30 (q, J=7.2 Hz, 2H), 1.93-1.90 (m, 2H), 1.57-1.54 (m, 2H), 1.09 (t, J=7.2 Hz, 3H). ESI-MS (m/z): 557.05 (M+H)⁺.

### Example 20

### Preparation of N-(3,5-dichloro-4-(1-(5-(6-methoxypyridin-3-yl)-1,2,4-oxadiazol-3-yl)cyclopropyl)phenyl)-2-(4-(ethylsulfonyl)phenyl)acetamide

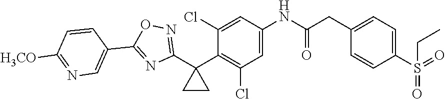 [SMILES: CCS(=O)(=O)c1ccc(CC(=O)Nc2cc(Cl)c(C3(c4noc(-c5ccc(OC)nc5)n4)CC3)c(Cl)c2)cc1]

Prepared using intermediate IV-1 and intermediate III-22. ¹H NMR (DMSO-d₆): 10.62 (s, 1H), 8.84 (d, J=1.6 Hz, 1H), 8.27 (dd, J=2.4 & 8.4 Hz, 1H), 7.86 (d, J=8.4 Hz, 2H), 7.75 (s, 2H), 7.61 (d, J=8.4 Hz, 2H), 7.03 (d, J=9.2 Hz, 1H), 3.95 (s, 3H), 3.84 (s, 2H), 3.31 (q, J=7.6 Hz, 2H), 1.91-1.88 (m, 2H), 1.54-1.51 (m, 2H), 1.09 (t, J=7.2 Hz, 3H). ESI-MS (m/z): 587.05 (M+H)⁺.

### Example 21

### Preparation of N-(3,5-dichloro-4-(1-(5-(4-fluorobenzyl)-1,2,4-oxadiazol-3-yl) cyclopropyl)phenyl)-2-(4-(ethylsulfonyl)phenyl)acetamide

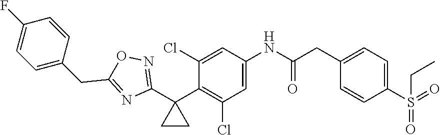 [SMILES: CCS(=O)(=O)c1ccc(CC(=O)Nc2cc(Cl)c(C3(c4noc(Cc5ccc(F)cc5)n4)CC3)c(Cl)c2)cc1]

Prepared using intermediate IV-1 and intermediate III-24. ¹H NMR (DMSO-d₆): 10.59 (s, 1H), 7.85 (d, J=8.4 Hz, 2H), 7.71 (s, 2H), 7.60 (d, J=8.4 Hz, 2H), 7.38-7.35 (m, 2H), 7.20-7.16 (m, 2H), 4.3 (s, 2H), 3.82 (s, 2H), 3.29 (q, J=7.2 Hz, 2H), 1.76-1.73 (m, 2H), 1.47-1.44 (m, 2H), 1.08 (t, J=7.2 Hz, 3H). ESI-MS (m/z): 586.05 (M−H).

### Example 22

### Preparation of N-(3,5-dichloro-4-(1-(5-(4-methoxyphenyl)-1,2,4-oxadiazol-3-yl)cyclopropyl)phenyl)-2-(4-(ethylsulfonyl)phenyl)acetamide

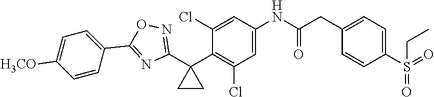 [SMILES: CCS(=O)(=O)c1ccc(CC(=O)Nc2cc(Cl)c(C3(c4noc(-c5ccc(OC)cc5)n4)CC3)c(Cl)c2)cc1]

Prepared using intermediate IV-1 and intermediate III-25. ¹H NMR (DMSO-d₆): 10.61 (s, 1H), 7.98 (dd, J=2.0 & 6.8 Hz, 2H), 7.86 (d, J=8.4 Hz, 2H), 7.74 (s, 2H), 7.61 (d, J=8.0 Hz, 2H), 7.14 (dd, J=2.4 & 7.2 Hz, 2H), 3.85 (s, 3H), 3.83 (s, 2H), 3.30 (q, J=7.2 Hz, 2H), 1.89-1.86 (m, 2H), 1.52-1.49 (m, 2H), 1.09 (t, J=7.2 Hz, 3H). ESI-MS (m/z): 586.10 (M+H)⁺.

### Example 23

### Preparation of N-(3,5-dichloro-4-(1-(5-isopropyl-1,2,4-oxadiazol-3-yl) cyclopropyl)phenyl)-2-(4-(ethylsulfonyl)phenyl)acetamide

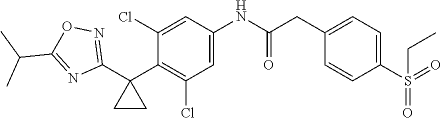 [SMILES: CCS(=O)(=O)c1ccc(CC(=O)Nc2cc(Cl)c(C3(c4noc(C(C)C)n4)CC3)c(Cl)c2)cc1]

Prepared using intermediate IV-1 and intermediate III-26. ¹H NMR (DMSO-d₆): 10.60 (s, 1H), 7.85 (dd, J=2 & 6.8 Hz, 2H), 7.71 (s, 2H), 7.60 (d, J=8.4 Hz, 2H), 3.82 (s, 2H), 3.30 (q, J=7.2 Hz, 2H), 3.24-3.17 (m, 1H), 1.78-1.74 (m, 2H), 1.46-1.43 (m, 2H), 1.27 (d, 6H), 1.09 (t, J=7.2 Hz, 3H). ESI-MS (m/z): 520.90 (M−H).

### Example 24

### Preparation of N-(4-(1-(1,2,4-oxadiazol-3-yl)cyclopropyl)-3,5-dichlorophenyl)-2-(4-(ethylsulfonyl)phenyl)acetamide

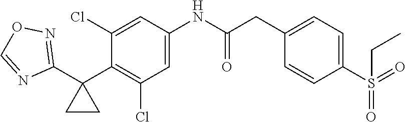 [SMILES: CCS(=O)(=O)c1ccc(CC(=O)Nc2cc(Cl)c(C3(c4ncon4)CC3)c(Cl)c2)cc1]

Prepared using intermediate IV-1 and intermediate III-27. ¹H NMR (DMSO-d₆): 10.60 (s, 1H), 9.44 (s, 1H), 7.85 (dd, J=1.6 & 6.4 Hz, 2H), 7.73 (s, 2H), 7.60 (d, J=8.4 Hz, 2H), 3.83 (s, 2H), 3.30 (q, J=7.2 Hz, 2H), 1.81-1.78 (m, 2H), 1.51-1.48 (m, 2H), 1.09 (t, J=7.2 Hz, 3H). ESI-MS (m/z): 478.0 (M−H).

### Example 25

### Preparation of N-(3,5-dichloro-4-(1-(5-cyclopropyl-1,2,4-oxadiazol-3-yl) cyclopropyl)phenyl)-2-(4-(ethylsulfonyl)phenyl)acetamide

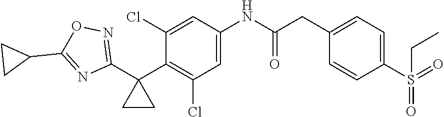 [SMILES: CCS(=O)(=O)c1ccc(CC(=O)Nc2cc(Cl)c(C3(c4noc(C5CC5)n4)CC3)c(Cl)c2)cc1]

Prepared using intermediate IV-1 and intermediate III-28. ¹H NMR (DMSO-d₆): 10.59 (s, 1H), 7.85 (d, J=8.4 Hz, 2H), 7.71 (s, 2H), 7.60 (d, J=8.4 Hz, 2H), 3.82 (s, 2H), 3.30 (q, J=7.6 Hz, 2H), 2.26-2.22 (m, 1H), 1.74-1.71 (m, 2H), 1.43-1.40 (m, 2H), 1.21-1.18 (m, 2H), 1.17-1.10 (m, 5H). ESI-MS (m/z): 520.65 (M+H)⁺.

### Example 26

### Preparation of N-(3,5-dichloro-4-(1-(5-(3,3-difluorocyclobutyl)-1,2,4-oxadiazol-3-yl)cyclopropyl)phenyl)-2-(4-(ethylsulfonyl)phenyl)acetamide

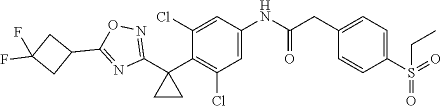 [SMILES: CCS(=O)(=O)c1ccc(CC(=O)Nc2cc(Cl)c(C3(c4noc(C5CC(F)(F)C5)n4)CC3)c(Cl)c2)cc1]

Prepared using intermediate IV-1 and intermediate III-29. ¹H NMR (DMSO-d₆): 10.60 (s, 1H), 7.85 (d, J=8.4 Hz, 2H), 7.72 (s, 2H), 7.60 (d, J=8.4 Hz, 2H), 3.83 (s, 2H), 3.74 (m, 1H), 3.30 (q, J=7.2 Hz, 2H), 3.11-3.06 (m, 2H), 2.96-2.90 (m, 2H), 1.80-1.77 (m, 2H), 1.49-1.48 (m, 2H), 1.09 (t, 3H). ESI-MS (m/z): 570.05 (M+H)⁺.

### Example 27

### Preparation of N-(3,5-dichloro-4-(1-(5-(tetrahydro-2H-pyran-4-yl)-1,2,4-oxadiazol-3-yl)cyclopropyl)phenyl)-2-(4-(ethylsulfonyl)phenyl)acetamide

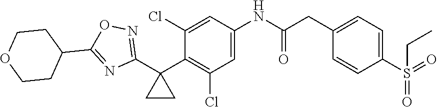 [SMILES: CCS(=O)(=O)c1ccc(CC(=O)Nc2cc(Cl)c(C3(c4noc(C5CCOCC5)n4)CC3)c(Cl)c2)cc1]

Prepared using intermediate IV-1 and intermediate III-30. ¹H NMR (DMSO-d₆): 10.59 (s, 1H), 7.85 (d, J=8.4 Hz, 2H), 7.72 (s, 2H), 7.60 (d, J=8.4 Hz, 2H), 3.86 (bd, 2H), 3.82 (s, 2H), 3.44-3.39 (m, 2H), 3.31-3.24 (m, 3H), 1.92 (bd, 2H), 1.78 (m, 2H), 1.71-1.66 (m, 2H), 1.47-1.44 (m, 2H), 1.09 (t, J=7.2 Hz, 3H). ESI-MS (m/z): 565.05 (M+H)⁺.

### Example 28

### Preparation of N-(3,5-difluoro-4-(1-(5-(4-fluorophenyl)-1,2,4-oxadiazol-3-yl) cyclopropyl)phenyl)-2-(4-(ethylsulfonyl)phenyl)acetamide

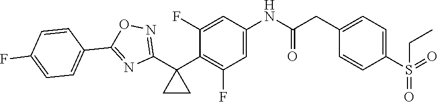 [SMILES: CCS(=O)(=O)c1ccc(CC(=O)Nc2cc(F)c(C3(c4noc(-c5ccc(F)cc5)n4)CC3)c(F)c2)cc1]

Prepared using intermediate IV-1 and intermediate III-31. ¹H NMR (DMSO-d₆): 10.65 (s, 1H), 8.11-8.07 (m, 2H), 7.85 (t, 2H), 7.61 (d, J=8.4 Hz, 2H), 7.46-7.42 (m, 2H), 7.35-7.32 (m, 2H), 3.84 (s, 2H), 3.30 (q, J=7.2 Hz, 2H), 1.73-1.70 (m, 2H), 1.45-1.42 (m, 2H), 1.09 (t, J=7.2 Hz, 3H). ESI-MS (m/z): 540.05 (M−H).

### Example 29

### Preparation of N-(3-chloro-5-fluoro-4-(1-(5-(4-fluorophenyl)-1,2,4-oxadiazol-3-yl)cyclopropyl)phenyl)-2-(4-(ethylsulfonyl)phenyl)acetamide

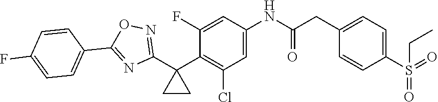 [SMILES: CCS(=O)(=O)c1ccc(CC(=O)Nc2cc(F)c(C3(c4noc(-c5ccc(F)cc5)n4)CC3)c(Cl)c2)cc1]

Prepared using intermediate IV-1 and intermediate III-32. ¹H NMR (DMSO-d₆): 10.65 (s, 1H), 8.12-8.08 (m, 2H), 7.86 (d, J=8.0 Hz, 2H), 7.61-7.57 (m, 3H), 7.53 (d, J=8.0 Hz, 1H), 7.44 (t, 2H), 3.84 (s, 2H), 3.30 (q, J=7.2 Hz, 2H), 1.79 (bs, 2H), 1.48 (bs, 2H), 1.09 (t, J=7.2 Hz, 3H). ESI-MS (m/z): 558.05 (M+H)⁺.

### Example 30

### Preparation of N-(3-chloro-4-(1-(5-(4-chlorophenyl)-1,2,4-oxadiazol-3-yl) cyclopropyl)-5-fluorophenyl)-2-(4-(ethylsulfonyl)phenyl)acetamide

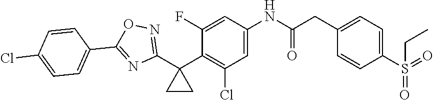 [SMILES: CCS(=O)(=O)c1ccc(CC(=O)Nc2cc(F)c(C3(c4noc(-c5ccc(Cl)cc5)n4)CC3)c(Cl)c2)cc1]

Prepared using intermediate IV-1 and intermediate III-33. ¹H NMR (DMSO-d₆): 10.65 (s, 1H), 8.05 (dd, J=2.0 & 6.8 Hz, 2H), 7.86 (d, J=8.4 Hz, 2H), 7.68 (m, 2H), 7.59 (m, 3H), 7.53 (m, 1H), 3.84 (s, 2H), 3.30 (q, J=7.6 Hz, 2H), 1.80 (bs, 2H), 1.48 (bs, 2H), 1.09 (t, J=7.2 Hz, 3H). ESI-MS (m/z): 574.05 (M+H)⁺.

### Example 31

### Preparation of N-(3-chloro-5-fluoro-4-(1-(5-(4-(trifluoromethyl)phenyl)-1,2,4-oxadiazol-3-yl)cyclopropyl)phenyl)-2-(4-(ethylsulfonyl)phenyl)acetamide

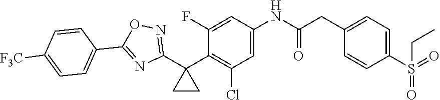 [SMILES: CCS(=O)(=O)c1ccc(CC(=O)Nc2cc(F)c(C3(c4noc(-c5ccc(C(F)(F)F)cc5)n4)CC3)c(Cl)c2)cc1]

Prepared using intermediate IV-1 and intermediate III-34. ¹H NMR (DMSO-d₆): 10.65 (s, 1H), 8.25 (d, J=8.0 Hz, 2H), 7.98 (d, J=8.4 Hz, 2H), 7.86 (dd, J=1.6 & 8.4 Hz, 2H), 7.61-7.53 (m, 3H), 7.51 (d, 1H), 3.84 (s, 2H), 3.30 (q, J=7.6 Hz, 2H), 1.82 (bs, 2H), 1.51 (bs, 2H), 1.09 (t, J=7.2 Hz, 3H). ESI-MS (m/z): 608.45 (M+H)⁺.

### Example 32

### Preparation of N-(3-chloro-4-(1-(5-(4-cyanophenyl)-1,2,4-oxadiazol-3-yl) cyclopropyl)-5-fluorophenyl)-2-(4-(ethylsulfonyl)phenyl)acetamide

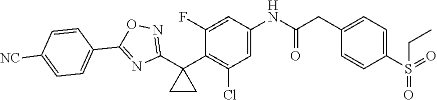 [SMILES: CCS(=O)(=O)c1ccc(CC(=O)Nc2cc(F)c(C3(c4noc(-c5ccc(C#N)cc5)n4)CC3)c(Cl)c2)cc1]

Prepared using intermediate IV-1 and intermediate III-35. ¹H NMR (DMSO-d₆): 10.65 (s, 1H), 8.20 (d, J=8.4 Hz, 2H), 8.08 (d, J=8.4 Hz, 2H), 7.86 (d, J=8.4 Hz, 2H), 7.61-7.58 (m, 3H), 7.54 (m, 1H), 3.84 (s, 2H), 3.30 (q, J=7.2 Hz, 2H), 1.81 (bs, 2H), 1.50 (bs, 2H), 1.09 (t, J=7.2 Hz, 3H). ESI-MS (m/z): 565.10 (M+H)⁺.

### Example 33

### Preparation of 2-(4-(ethylsulfonyl)phenyl)-N-(4-(1-(5-(4-fluorophenyl)-1,2,4-oxadiazol-3-yl)cyclopropyl)-3,5-dimethylphenyl)acetamide

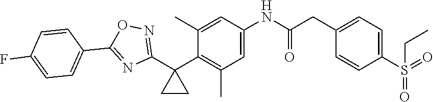 [SMILES: CCS(=O)(=O)c1ccc(CC(=O)Nc2cc(C)c(C3(c4noc(-c5ccc(F)cc5)n4)CC3)c(C)c2)cc1]

Prepared using intermediate IV-1 and intermediate III-36. ¹H NMR (DMSO-d₆): 10.15 (s, 1H), 8.12-8.08 (m, 2H), 7.85 (d, J=8.0 Hz, 2H), 7.60 (d, J=8.4 Hz, 2H), 7.46-7.42 (m, 2H), 7.27 (s, 2H), 3.77 (s, 2H), 3.29 (q, J=7.2 Hz, 2H), 2.27 (s, 6H), 1.76-1.73 (m, 2H), 1.32-1.30 (m, 2H), 1.10 (t, J=7.2 Hz, 3H). ESI-MS (m/z): 534.15 (M+H)⁺.

### Example 34

### Preparation of N-(3,5-dichloro-4-(1-(5-(4-(trifluoromethoxy)phenyl)-1,2,4-oxadiazol-3-yl)cyclopropyl)phenyl)-2-(4-(ethylsulfonyl)phenyl)acetamide

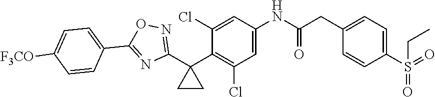 [SMILES: CCS(=O)(=O)c1ccc(CC(=O)Nc2cc(Cl)c(C3(c4noc(-c5ccc(OC(F)(F)F)cc5)n4)CC3)c(Cl)c2)cc1]

Prepared using intermediate IV-1 and intermediate III-37. ¹H NMR (DMSO-d₆): 10.63 (s, 1H), 8.18 (dd, J=2.0 & 6.8 Hz, 2H), 7.86 (d, J=8.4 Hz, 2H), 7.75 (s, 2H), 7.61-7.58 (m, 4H), 3.84 (s, 2H), 3.30 (q, J=7.2 Hz, 2H), 1.91-1.88 (m, 2H), 1.56-1.53 (m, 2H), 1.09 (t, J=7.2 Hz, 3H). ESI-MS (m/z): 640.00 (M+H)⁺.

### Example 35

### Preparation of N-(3,5-dichloro-4-(1-(3-(4-fluorophenyl)-1,2,4-oxadiazol-5-yl)cyclopropyl)phenyl)-2-(4-(ethylsulfonyl)phenyl)acetamide

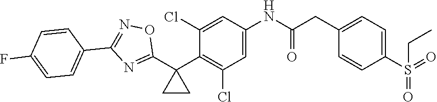 [SMILES: CCS(=O)(=O)c1ccc(CC(=O)Nc2cc(Cl)c(C3(c4nc(-c5ccc(F)cc5)no4)CC3)c(Cl)c2)cc1]

Prepared using intermediate IV-1 and intermediate III-38. ¹H NMR (DMSO-d₆): 10.64 (s, 1H), 7.97-7.90 (m, 2H), 7.86 (d, J=8.0 Hz, 2H), 7.79 (s, 2H), 7.61 (d, J=8.4 Hz, 2H), 7.36 (t, 2H), 3.84 (s, 2H), 3.30 (q, J=7.2 Hz, 2H), 2.13-2.09 (m, 2H), 1.76-1.72 (m, 2H), 1.09 (t, J=7.6 Hz, 3H). ESI-MS (m/z): 574.00 (M+H)⁺.

### Example 36

### Preparation of N-(3,5-dichloro-4-(1-(6-fluorobenzo[d]oxazol-2-yl)cyclopropyl) phenyl)-2-(4-(ethylsulfonyl)phenyl)acetamide

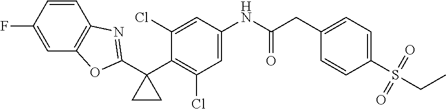 [SMILES: CCS(=O)(=O)c1ccc(CC(=O)Nc2cc(Cl)c(C3(c4nc5ccc(F)cc5o4)CC3)c(Cl)c2)cc1]

Prepared using intermediate IV-1 and intermediate III-39. ¹H NMR (DMSO-d₆): 10.64 (s, 1H), 7.86 (d, J=8 Hz, 2H), 7.76 (s, 2H), 7.63-7.59 (m, 4H), 7.2-7.1 (m, 1H), 3.84 (s, 2H), 3.30 (q, J=7.6 Hz, 2H), 2.06-2.02 (m, 2H), 1.64-1.60 (m, 2H), 1.09 (t, J=7.6 Hz, 3H). ESI-MS (m/z): 547.65 (M+H)⁺.

### Example 37

### Preparation of 2-(4-(ethylsulfonyl)phenyl)-N-(4-(1-(6-fluorobenzo[d]oxazol-2-yl)cyclopropyl)-3,5-dimethylphenyl)acetamide

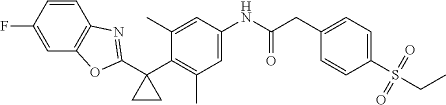 [SMILES: CCS(=O)(=O)c1ccc(CC(=O)Nc2cc(C)c(C3(c4nc5ccc(F)cc5o4)CC3)c(C)c2)cc1]

Prepared using intermediate IV-1 and intermediate III-40. ¹H NMR (DMSO-d₆): 10.17 (s, 1H), 7.86 (d, J=8.0 Hz, 2H), 7.61-7.57 (m, 4H), 7.29 (s, 2H), 7.19-7.16 (m, 1H), 3.78 (s, 2H), 3.30 (q, J=7.2 Hz, 2H), 2.50 (s, 6H), 1.91-1.89 (m, 2H), 1.42-1.39 (m, 2H), 1.09 (t, J=7.2 Hz, 3H). ESI-MS (m/z): 507.15 (M+H)⁺.

### Example 38

### Preparation of N-(3,5-dichloro-4-(1-(5-(4-fluorophenyl)oxazol-2-yl)cyclopropyl) phenyl)-2-(4-(ethylsulfonyl)phenyl)acetamide

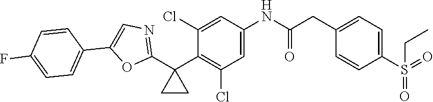 [SMILES: CCS(=O)(=O)c1ccc(CC(=O)Nc2cc(Cl)c(C3(c4ncc(-c5ccc(F)cc5)o4)CC3)c(Cl)c2)cc1]

Prepared using intermediate IV-1 and intermediate III-41. ¹H NMR (DMSO-d₆): 10.62 (s, 1H), 7.86 (d, J=8.4 Hz, 2H), 7.75 (s, 2H), 7.61-7.57 (m, 4H), 7.44 (s, 1H), 7.27 (t, 2H), 3.84 (s, 2H), 3.28 (q, J=7.2 Hz, 2H), 1.94-1.91 (m, 2H), 1.50-1.47 (m, 2H), 1.09 (t, J=7.2 Hz, 3H). ESI-MS (m/z): 573.05 (M+H)⁺.

### Example 39

### Preparation of N-(3,5-dichloro-4-(1-(5-(4-chlorophenyl)oxazol-2-yl)cyclopropyl) phenyl)-2-(4-(ethylsulfonyl)phenyl)acetamide

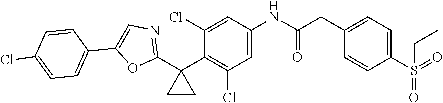 [SMILES: CCS(=O)(=O)c1ccc(CC(=O)Nc2cc(Cl)c(C3(c4ncc(-c5ccc(Cl)cc5)o4)CC3)c(Cl)c2)cc1]

Prepared using intermediate IV-1 and intermediate III-42. ¹H NMR (DMSO-d₆): 10.66 (s, 1H), 7.86 (d, J=8.4 Hz, 2H), 7.76 (s, 2H), 7.61-7.48 (m, 4H+3H), 3.84 (s, 2H), 3.28 (q, J=7.6 Hz, 2H), 1.93 (m, 2H), 1.50 (m, 2H), 1.11 (t, 3H). ESI-MS (m/z): 589.95 (M+H)⁺.

### Example 40

### Preparation of N-(3,5-dichloro-4-(1-(5-(3-fluoro-4-methoxyphenyl)oxazol-2-yl) cyclopropyl)phenyl)-2-(4-(ethylsulfonyl)phenyl)acetamide

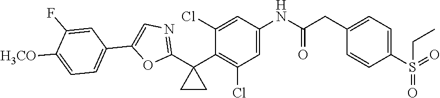 [SMILES: CCS(=O)(=O)c1ccc(CC(=O)Nc2cc(Cl)c(C3(c4ncc(-c5ccc(OC)c(F)c5)o4)CC3)c(Cl)c2)cc1]

Prepared using intermediate IV-1 and intermediate III-43. ¹H NMR (DMSO-d₆): 10.62 (s, 1H), 7.86 (d, J=8.4 Hz, 2H), 7.75 (s, 2H), 7.61 (d, J=8.4 Hz, 2H), 7.47 (dd, J=2.0 & 12.4 Hz, 1H), 7.38 (s, 1H), 7.33 (d, J=8.4 Hz, 1H), 7.22 (t, 1H), 3.847 (s, 3H), 3.840 (s, 2H), 3.30 (q, J=7.2 Hz, 2H), 1.94-1.91 (m, 2H), 1.49-1.46 (m, 2H), 1.09 (t, J=7.2 Hz, 3H). ESI-MS (m/z): 603.60 (M+H)⁺.

### Example 41

### Preparation of N-(3,5-dichloro-4-(1-(5-(4-fluorophenyl)-1,2,4-oxadiazol-3-yl) cyclopropyl)phenyl)-2-(5-(ethylsulfonyl)pyridin-2-yl)acetamide

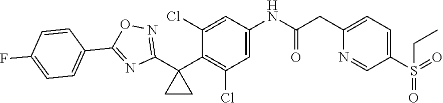 [SMILES: CCS(=O)(=O)c1ccc(CC(=O)Nc2cc(Cl)c(C3(c4noc(-c5ccc(F)cc5)n4)CC3)c(Cl)c2)nc1]

Prepared using intermediate IV-2 and intermediate III-1. ¹H NMR (DMSO-d₆): 10.69 (s, 1H), 8.96 (d, J=2 Hz, 1H), 8.28 (dd, J=2.4 & 8.0 Hz, 1H), 8.12-8.09 (m, 2H), 7.75 (s, 2H), 7.72 (d, J=8.4 Hz, 1H), 7.45 (t, 2H), 4.05 (s, 2H), 3.43 (q, J=7.2 Hz, 2H), 1.91-1.88 (m, 2H), 1.55-1.52 (m, 2H), 1.13 (t, J=7.2 Hz, 3H). ESI-MS (m/z): 575.05 (M+H)⁺.

### Example 42

### Preparation of N-(3,5-dichloro-4-(1-(5-(4-(trifluoromethyl)phenyl)-1,2,4-oxadiazol-3-yl)cyclopropyl)phenyl)-2-(5-(ethylsulfonyl)pyridin-2-yl)acetamide

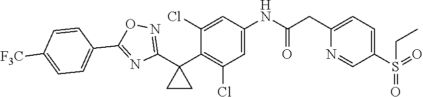 [SMILES: CCS(=O)(=O)c1ccc(CC(=O)Nc2cc(Cl)c(C3(c4noc(-c5ccc(C(F)(F)F)cc5)n4)CC3)c(Cl)c2)nc1]

Prepared using intermediate IV-2 and intermediate III-3. ¹H NMR (DMSO-d₆): 10.69 (s, 1H), 8.96 (d, J=2 Hz, 1H), 8.28-8.24 (m, 3H), 7.99 (d, J=8.4 Hz, 2H), 7.76 (s, 2H), 7.72 (d, J=8.0 Hz, 1H), 4.05 (s, 2H), 3.43 (q, J=7.2 Hz, 2H), 1.94-1.90 (m, 2H), 1.58-1.55 (m, 2H), 1.13 (t, J=7.2 Hz, 3H). ESI-MS (m/z): 625.60 (M+H)⁺.

### Example 43

### Preparation of N-(3,5-dichloro-4-(1-(5-(p-tolyl)-1,2,4-oxadiazol-3-yl)cyclopropyl) phenyl)-2-(5-(ethylsulfonyl)pyridin-2-yl)acetamide

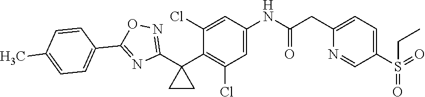 [SMILES: CCS(=O)(=O)c1ccc(CC(=O)Nc2cc(Cl)c(C3(c4noc(-c5ccc(C)cc5)n4)CC3)c(Cl)c2)nc1]

Prepared using intermediate IV-2 and intermediate III-4. ¹H NMR (DMSO-d₆): 10.69 (s, 1H), 8.96 (d, J=2 Hz, 1H), 8.28 (dd, J=2.4 & 8.0 Hz, 1H), 7.93 (d, J=8.4 Hz, 2H), 7.75 (s, 2H), 7.72 (d, J=8.0 Hz, 1H), 7.42 (d, J=8.4 Hz, 2H), 4.05 (s, 2H), 3.41 (q, J=7.6 Hz, 2H), 2.4 (s, 3H), 1.89-1.88 (m, 2H), 1.53-1.52 (m, 2H), 1.13 (t, J=7.6 Hz, 3H). ESI-MS (m/z): 571.05 (M+H)⁺.

### Example 44

### Preparation of N-(3,5-dichloro-4-(1-(5-(3,4-difluorophenyl)-1,2,4-oxadiazol-3-yl) cyclopropyl)phenyl)-2-(5-(ethylsulfonyl)pyridin-2-yl)acetamide

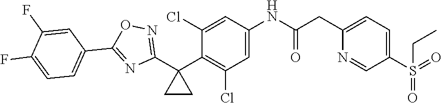 [SMILES: CCS(=O)(=O)c1ccc(CC(=O)Nc2cc(Cl)c(C3(c4noc(-c5ccc(F)c(F)c5)n4)CC3)c(Cl)c2)nc1]

Prepared using intermediate IV-2 and intermediate III-5. ¹H NMR (DMSO-d₆): 10.69 (s, 1H), 8.96 (d, J=2.0 Hz, 1H), 8.28 (dd, J=2.4 & 8.0 Hz, 1H), 8.12-8.10 (m, 1H), 7.94 (bs, 1H), 7.75 (s, 2H), 7.72-7.68 (m, 2H), 4.05 (s, 2H), 3.43 (q, J=7.6 Hz, 2H), 1.91-1.88 (m, 2H), 1.56-1.53 (m, 2H), 1.13 (t, J=7.2 Hz, 3H). ESI-MS (m/z): 593.00 (M+H)⁺.

### Example 45

### Preparation of N-(3,5-dichloro-4-(1-(5-(3,4,5-trifluorophenyl)-1,2,4-oxadiazol-3-yl)cyclopropyl)phenyl)-2-(5-(ethylsulfonyl)pyridin-2-yl)acetamide

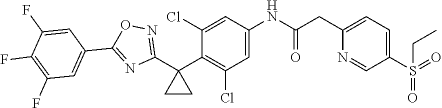 [SMILES: CCS(=O)(=O)c1ccc(CC(=O)Nc2cc(Cl)c(C3(c4noc(-c5cc(F)c(F)c(F)c5)n4)CC3)c(Cl)c2)nc1]

Prepared using intermediate IV-2 and intermediate III-23. ¹H NMR (DMSO-d₆): 10.69 (s, 1H), 8.96 (d, J=2 Hz, 1H), 8.28 (dd, J=2.4 & 8.4 Hz, 1H), 8.03 (m, 2H), 7.76 (s, 2H), 7.72 (d, J=8.4 Hz, 1H), 4.05 (s, 2H), 3.43 (q, J=7.6 Hz, 2H), 1.92-1.89 (m, 2H), 1.57-1.54 (m, 2H), 1.15 (t, J=7.6 Hz, 3H). ESI-MS (m/z): 611.00 (M+H)⁺.

### Example 46

### Preparation of N-(3,5-dichloro-4-(1-(5-(2-chloro-4-fluorophenyl)-1,2,4-oxadiazol-3-yl)cyclopropyl)phenyl)-2-(5-(ethylsulfonyl)pyridin-2-yl)acetamide

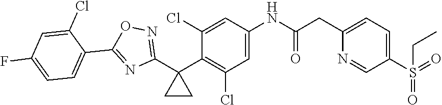 [SMILES: CCS(=O)(=O)c1ccc(CC(=O)Nc2cc(Cl)c(C3(c4noc(-c5ccc(F)cc5Cl)n4)CC3)c(Cl)c2)nc1]

Prepared using intermediate IV-2 and intermediate III-9. ¹H NMR (DMSO-d₆): 10.70 (s, 1H), 8.96 (d, J=2.0 Hz, 1H), 8.28 (dd, J=2.4 & 8.4 Hz, 1H), 8.15 (dd, J=6 & 8.8 Hz, 1H), 7.78 (d, J=2.4 Hz, 1H), 7.76 (s, 2H), 7.72 (d, J=8.0 Hz, 1H), 7.49-7.46 (m, 1H), 4.05 (s, 2H), 3.43 (q, J=7.2 Hz, 2H), 1.91-1.88 (m, 2H), 1.57-1.54 (m, 2H), 1.13 (t, J=7.2 Hz, 3H). ESI-MS (m/z): 610.45 (M−H).

### Example 47

### Preparation of N-(3,5-dichloro-4-(1-(5-(3-chloro-4-fluorophenyl)-1,2,4-oxadiazol-3-yl)cyclopropyl)phenyl)-2-(5-(ethylsulfonyl)pyridin-2-yl)acetamide

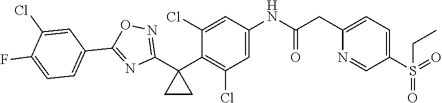 [SMILES: CCS(=O)(=O)c1ccc(CC(=O)Nc2cc(Cl)c(C3(c4noc(-c5ccc(F)c(Cl)c5)n4)CC3)c(Cl)c2)nc1]

Prepared using intermediate IV-2 and intermediate III-10. ¹H NMR (DMSO-d₆): 10.69 (s, 1H), 8.96 (d, J=2.0 Hz, 1H), 8.28 (dd, J=2.4 & 8.4 Hz, 1H), 8.24 (dd, J=2.0 & 6.8 Hz, 1H), 8.2-8.1 (m, 1H), 7.76 (s, 2H), 7.72-7.66 (m, 2H), 4.05 (s, 2H), 3.41 (q, J=7.6 Hz, 2H), 1.91 (bd, 2H), 1.55 (bd, 2H), 1.13 (t, J=7.2 Hz, 3H). ESI-MS (m/z): 609.00 (M+H)⁺.

### Example 48

### Preparation of N-(3,5-dichloro-4-(1-(5-(3,4-dichlorophenyl)-1,2,4-oxadiazol-3-yl)cyclopropyl)phenyl)-2-(5-(ethylsulfonyl)pyridin-2-yl)acetamide

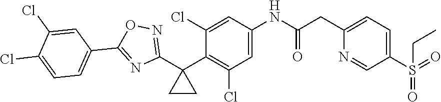 [SMILES: CCS(=O)(=O)c1ccc(CC(=O)Nc2cc(Cl)c(C3(c4noc(-c5ccc(Cl)c(Cl)c5)n4)CC3)c(Cl)c2)nc1]

Prepared using intermediate IV-2 and intermediate III-11. ¹H NMR (DMSO-d₆): 10.69 (s, 1H), 8.96 (d, J=2.4 Hz, 1H), 8.28 (dd, J=2.4 & 8.4 Hz, 1H), 8.23 (d, J=2.0 Hz, 1H), 8.01 (dd, J=2.0 & 8.4 Hz, 1H), 7.89 (d, J=8.4 Hz, 1H), 7.76 (s, 2H), 7.72 (d, J=8.0 Hz, 1H), 4.05 (s, 2H), 3.41 (q, J=7.6 Hz, 2H), 1.91 (bd, 2H), 1.56 (bd, 2H), 1.13 (t, J=7.6 Hz, 3H). ESI-MS (m/z): 627.00 (M+H)⁺.

### Example 49

### Preparation of N-(3,5-dichloro-4-(1-(5-(2,4-dichlorophenyl)-1,2,4-oxadiazol-3-yl)cyclopropyl)phenyl)-2-(5-(ethylsulfonyl)pyridin-2-yl)acetamide

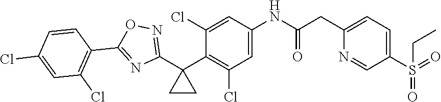 [SMILES: CCS(=O)(=O)c1ccc(CC(=O)Nc2cc(Cl)c(C3(c4noc(-c5ccc(Cl)cc5Cl)n4)CC3)c(Cl)c2)nc1]

Prepared using intermediate IV-2 and intermediate III-12. ¹H NMR (DMSO-d₆): 10.69 (s, 1H), 8.96 (d, J=2.0 Hz, 1H), 8.26 (d, J=2.4 Hz, 1H), 8.08 (d, J=8.8 Hz, 1H), 7.94 (d, J=2.0 Hz, 1H), 7.76 (s, 2H), 7.72 (d, J=8.4 Hz, 1H), 7.67 (dd, J=2.0 & 8.4 Hz, 1H), 4.05 (s, 2H), 3.41 (q, J=7.6 Hz, 2H), 1.90 (bd, 2H), 1.56 (bd, 2H), 1.13 (t, J=7.6 Hz, 3H). ESI-MS (m/z): 626.95 (M+H)⁺.

### Example 50

### Preparation of N-(3,5-dichloro-4-(1-(5-(4-chlorophenyl)-1,2,4-oxadiazol-3-yl)cyclopropyl)phenyl)-2-(5-(ethylsulfonyl)pyridin-2-yl)acetamide

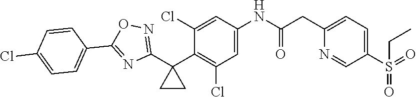 [SMILES: CCS(=O)(=O)c1ccc(CC(=O)Nc2cc(Cl)c(C3(c4noc(-c5ccc(Cl)cc5)n4)CC3)c(Cl)c2)nc1]

Prepared using intermediate IV-2 and intermediate III-13. ¹H NMR (DMSO-d₆): 10.68 (s, 1H), 8.96 (d, J=2.0 Hz, 1H), 8.3-8.2 (m, 1H), 8.06 (d, J=8.8 Hz, 2H), 7.75 (s, 2H), 7.72-7.67 (m, 3H), 4.05 (s, 2H), 3.41 (q, J=7.2 Hz, 2H), 1.90 (bd, 2H), 1.55 (bd, 2H), 1.13 (t, J=7.6 Hz, 3H). ESI-MS (m/z): 592.95 (M+H)⁺.

### Example 51

### Preparation of N-(3,5-dichloro-4-(1-(5-(3-chlorophenyl)-1,2,4-oxadiazol-3-yl) cyclopropyl)phenyl)-2-(5-(ethylsulfonyl)pyridin-2-yl)acetamide

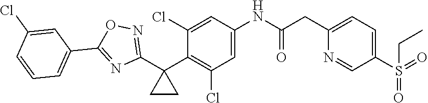 [SMILES: CCS(=O)(=O)c1ccc(CC(=O)Nc2cc(Cl)c(C3(c4noc(-c5cccc(Cl)c5)n4)CC3)c(Cl)c2)nc1]

Prepared using intermediate IV-2 and intermediate III-14. ¹H NMR (DMSO-d₆): 10.69 (s, 1H), 8.96 (d, J=2.4 Hz, 1H), 8.28 (dd, J=2.4 & 8.4 Hz, 1H), 8.03 (d, J=1.6 Hz, 1H), 8.01 (d, J=8.0 Hz, 1H), 7.78 (bs, 1H), 7.76 (s, 2H), 7.72 (d, J=8.4 Hz, 1H), 7.66-7.62 (m, 1H), 4.05 (s, 2H), 3.43 (q, J=7.2 Hz, 2H), 1.92-1.89 (m, 2H), 1.56-1.53 (m, 2H), 1.13 (t, J=7.6 Hz, 3H). ESI-MS (m/z): 593.00 (M+H)⁺.

### Example 52

### Preparation of N-(3,5-dichloro-4-(1-(5-(4-methoxyphenyl)-1,2,4-oxadiazol-3-yl) cyclopropyl)phenyl)-2-(5-(ethylsulfonyl)pyridin-2-yl)acetamide

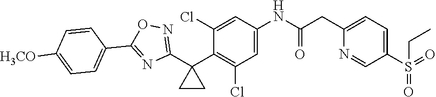 [SMILES: CCS(=O)(=O)c1ccc(CC(=O)Nc2cc(Cl)c(C3(c4noc(-c5ccc(OC)cc5)n4)CC3)c(Cl)c2)nc1]

Prepared using intermediate IV-2 and intermediate III-15. ¹H NMR (DMSO-d₆): 10.68 (s, 1H), 8.96 (d, J=2.4 Hz, 1H), 8.26 (d, J=2.4 Hz, 1H), 7.99 (d, J=8.8 Hz, 2H), 7.75 (s, 2H), 7.72 (d, J=8.0 Hz, 1H), 7.14 (d, J=8.8 Hz, 2H), 4.05 (s, 2H), 3.85 (s, 3H), 3.41 (q, J=7.2 Hz, 2H), 1.88 (bd, 2H), 1.52 (bd, 2H), 1.13 (t, J=7.2 Hz, 3H). ESI-MS (m/z): 587.05 (M+H)⁺.

### Example 53

### Preparation of N-(3,5-dichloro-4-(1-(5-(4-cyanophenyl)-1,2,4-oxadiazol-3-yl) cyclopropyl)phenyl)-2-(5-(ethylsulfonyl)pyridin-2-yl)acetamide

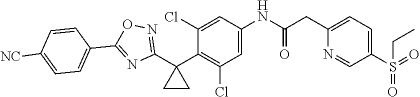 [SMILES: CCS(=O)(=O)c1ccc(CC(=O)Nc2cc(Cl)c(C3(c4noc(-c5ccc(C#N)cc5)n4)CC3)c(Cl)c2)nc1]

Prepared using intermediate IV-2 and intermediate III-16. ¹H NMR (DMSO-d₆): 10.69 (s, 1H), 8.96 (d, J=1.6 Hz, 1H), 8.26 (m, 1H), 8.21 (d, J=8.4 Hz, 2H), 8.08 (d, J=8.4 Hz, 2H), 7.76 (s, 2H), 7.72 (d, J=8.0 Hz, 1H), 4.05 (s, 2H), 3.41 (q, J=7.6 Hz, 2H), 1.91 (bd, 2H), 1.57 (bd, 2H), 1.13 (t, J=7.6 Hz, 3H). ESI-MS (m/z): 582.05 (M+H)⁺.

### Example 54

### Preparation of N-(3,5-dichloro-4-(1-(5-(5-chlorothiophen-2-yl)-1,2,4-oxadiazol-3-yl)cyclopropyl)phenyl)-2-(5-(ethylsulfonyl)pyridin-2-yl)acetamide

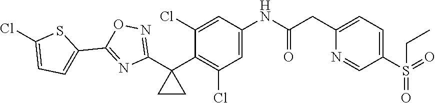 [SMILES: CCS(=O)(=O)c1ccc(CC(=O)Nc2cc(Cl)c(C3(c4noc(-c5ccc(Cl)s5)n4)CC3)c(Cl)c2)nc1]

Prepared using intermediate IV-2 and intermediate III-19. ¹H NMR (DMSO-d₆): 10.68 (s, 1H), 8.96 (d, J=2.0 Hz, 1H), 8.28 (dd, J=2.4 & 8.0 Hz, 1H), 7.87 (d, J=4.0 Hz, 1H), 7.75 (s, 2H), 7.72 (d, J=8.0 Hz, 1H), 7.38 (d, J=4.0 Hz, 1H), 4.04 (s, 2H), 3.43 (q, J=7.2 Hz, 2H), 1.86-1.83 (m, 2H), 1.54-1.51 (m, 2H), 1.13 (t, J=7.2 Hz, 3H). ESI-MS (m/z): 598.95 (M+H)⁺.

### Example 55

### Preparation of N-(3,5-dichloro-4-(1-(5-(3,3-difluorocyclobutyl)-1,2,4-oxadiazol-3-yl)cyclopropyl)phenyl)-2-(5-(ethylsulfonyl)pyridin-2-yl)acetamide

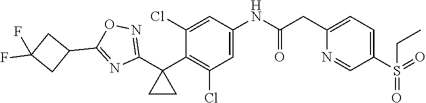 [SMILES: CCS(=O)(=O)c1ccc(CC(=O)Nc2cc(Cl)c(C3(c4noc(C5CC(F)(F)C5)n4)CC3)c(Cl)c2)nc1]

Prepared using intermediate IV-2 and intermediate III-29. ¹H NMR (DMSO-d₆): 10.67 (s, 1H), 8.95 (d, J=2.0 Hz, 1H), 8.27 (dd, J=2.4 & 8.0 Hz, 1H), 7.73 (s, 2H), 7.71 (d, J=8.4 Hz, 1H), 4.04 (s, 2H), 3.73-3.71 (m, 1H), 3.41 (q, J=7.2 Hz, 2H), 3.13-3.06 (m, 2H), 3.0-2.90 (m, 2H), 1.80-1.78 (m, 2H), 1.77-1.49 (m, 2H), 1.13 (t, J=7.2 Hz, 3H). ESI-MS (m/z): 571.05 (M+H)⁺.

### Example 56

### Preparation of N-(3,5-dichloro-4-(1-(5-cyclopropyl-1,2,4-oxadiazol-3-yl) cyclopropyl)phenyl)-2-(5-(ethylsulfonyl)pyridin-2-yl)acetamide

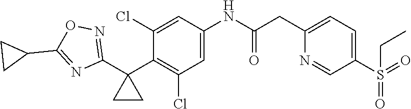 [SMILES: CCS(=O)(=O)c1ccc(CC(=O)Nc2cc(Cl)c(C3(c4noc(C5CC5)n4)CC3)c(Cl)c2)nc1]

Prepared using intermediate IV-2 and intermediate III-28. ¹H NMR (DMSO-d₆): 10.64 (s, 1H), 8.95 (d, J=2.4 Hz, 1H), 8.27 (dd, J=2.8 & 8.4 Hz, 1H), 7.71 (s, 2H), 7.69 (d, 1H), 4.03 (s, 2H), 3.42 (q, J=7.2 Hz, 2H), 2.24 (m, 1H), 1.73-1.72 (m, 2H), 1.43-1.42 (m, 2H), 1.20-1.17 (m, 2H), 1.14 (t, J=7.2 Hz, 3H), 1.05-1.04 (m, 2H). ESI-MS (m/z): 520.90 (M−H).

### Example 57

### Preparation of 2-(5-(ethylsulfonyl)pyridin-2-yl)-N-(4-(1-(5-(4-fluorophenyl)-1,2,4-oxadiazol-3-yl)cyclopropyl)-3,5-dimethylphenyl)acetamide

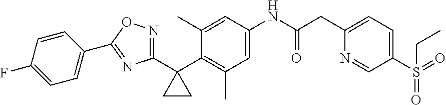 [SMILES: CCS(=O)(=O)c1ccc(CC(=O)Nc2cc(C)c(C3(c4noc(-c5ccc(F)cc5)n4)CC3)c(C)c2)nc1]

Prepared using intermediate IV-2 and intermediate III-36. ¹H NMR (DMSO-d₆): 10.20 (s, 1H), 8.95 (d, J=1.6 Hz, 1H), 8.27 (dd, J=2.4 & 8.4 Hz, 1H), 8.12-8.08 (m, 2H), 7.70 (d, J=8.4 Hz, 1H), 7.44 (t, 2H), 7.28 (s, 2H), 3.99 (s, 2H), 3.42 (q, J=7.2 Hz, 2H), 2.27 (s, 6H), 1.76-1.73 (m, 2H), 1.33-1.32 (m, 2H), 1.12 (t, J=7.2 Hz, 3H). ESI-MS (m/z): 535.15 (M+H)⁺.

### Example 58

### Preparation of N-(3,5-dichloro-4-(1-(6-fluorobenzo[d]oxazol-2-yl)cyclopropyl) phenyl)-2-(5-(ethylsulfonyl)pyridin-2-yl)acetamide

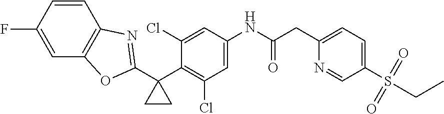 [SMILES: CCS(=O)(=O)c1ccc(CC(=O)Nc2cc(Cl)c(C3(c4nc5ccc(F)cc5o4)CC3)c(Cl)c2)nc1]

Prepared using intermediate IV-2 and intermediate III-39. ¹H NMR (DMSO-d₆): 10.70 (s, 1H), 8.96 (d, J=2.0 Hz, 1H), 8.27 (d, J=2.4 Hz, 1H), 7.77 (s, 2H), 7.72 (d, J=8.0 Hz, 1H), 7.63-7.60 (m, 2H), 7.1-7.2 (m, 1H), 4.06 (s, 2H), 3.41 (q, J=7.2 Hz, 2H), 2.05-2.04 (bd, 2H), 1.63 (bd, 2H), 1.13 (t, J=7.2 Hz, 3H). ESI-MS (m/z): 548.00 (M+H)⁺.

### Example 59

### Preparation of 2-(5-(ethylsulfonyl)pyridin-2-yl)-N-(4-(1-(6-fluorobenzo[d]oxazol-2-yl)cyclopropyl)-3,5-dimethylphenyl)acetamide

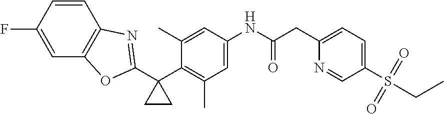 [SMILES: CCS(=O)(=O)c1ccc(CC(=O)Nc2cc(C)c(C3(c4nc5ccc(F)cc5o4)CC3)c(C)c2)nc1]

Prepared using intermediate IV-2 and intermediate III-40. ¹H NMR (DMSO-d₆): 10.22 (s, 1H), 8.96 (d, J=2.0 Hz, 1H), 8.28 (dd, J=2.4 & 8.4 Hz, 1H), 7.71 (d, J=8.4 Hz, 1H), 7.61-7.57 (m, 2H), 7.30 (s, 2H), 7.19-7.14 (m, 1H), 4.01 (s, 2H), 3.43 (q, J=7.6 Hz, 2H), 2.28 (s, 6H), 1.92-1.89 (m, 2H), 1.43-1.42 (m, 2H), 1.13 (t, J=7.2 Hz, 3H). ESI-MS (m/z): 508.15 (M+H)⁺.

### Example 60

### Preparation of N-(3,5-dichloro-4-(1-(5-(4-fluorophenyl)oxazol-2-yl)cyclopropyl) phenyl)-2-(5-(ethylsulfonyl)pyridin-2-yl)acetamide

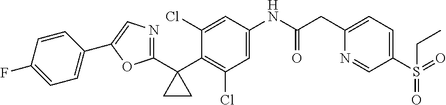 [SMILES: CCS(=O)(=O)c1ccc(CC(=O)Nc2cc(Cl)c(C3(c4ncc(-c5ccc(F)cc5)o4)CC3)c(Cl)c2)nc1]

Prepared using intermediate IV-2 and intermediate III-41. ¹H NMR (DMSO-d₆): 10.69 (s, 1H), 8.96 (s, 1H), 8.28 (d, J=6.4 Hz, 1H), 7.76 (s, 2H), 7.72 (d, J=6.4 Hz, 1H), 7.62-7.58 (m, 2H), 7.44 (s, 1H), 7.28 (t, 2H), 4.05 (s, 2H), 3.43 (q, J=6.8 Hz, 2H), 1.93 (m, 2H), 1.50 (m, 2H), 1.13 (t, J=7.2 Hz, 3H). ESI-MS (m/z): 574.10 (M+H)⁺.

### Example 61

### Preparation of N-(3,5-dichloro-4-(1-(5-(4-fluorophenyl)-1,2,4-oxadiazol-3-yl) cyclopropyl)phenyl)-2-(4-(ethylsulfonimidoyl)phenyl)acetamide

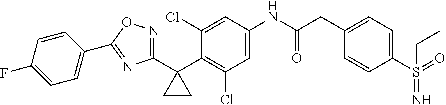 [SMILES: CCS(=N)(=O)c1ccc(CC(=O)Nc2cc(Cl)c(C3(c4noc(-c5ccc(F)cc5)n4)CC3)c(Cl)c2)cc1]

### Step 1: benzyl ((4-(2-((3,5-dichloro-4-(1-(5-(4-fluorophenyl)-1,2,4-oxadiazol-3-yl) cyclopropyl)phenyl)amino)-2-oxoethyl)phenyl)(ethyl)(oxo)-γ⁶-sulfanylidene)carbamate

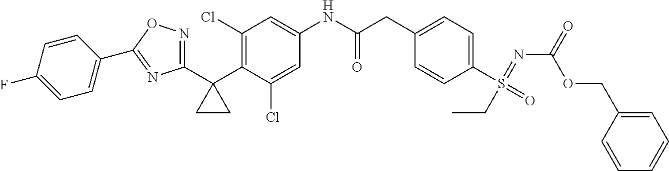 [SMILES: CCS(=O)(=NC(=O)OCc1ccccc1)c1ccc(CC(=O)Nc2cc(Cl)c(C3(c4noc(-c5ccc(F)cc5)n4)CC3)c(Cl)c2)cc1]

Prepared using intermediate IV-3 and intermediate III-1 using similar procedure as described for example 1 and directly used for next step.

### Step 2: N-(3,5-dichloro-4-(1-(5-(4-fluorophenyl)-1,2,4-oxadiazol-3-yl)cyclopropyl) phenyl)-2-(4-(ethylsulfonimidoyl)phenyl)acetamide

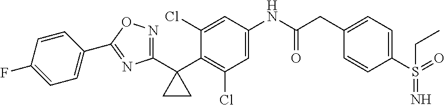 [SMILES: CCS(=N)(=O)c1ccc(CC(=O)Nc2cc(Cl)c(C3(c4noc(-c5ccc(F)cc5)n4)CC3)c(Cl)c2)cc1]

Product of step 1 (250 mg, 0.353 mmol) was slowly added to sulfuric acid (1 mL) at 0° C. and stirred for 1 h at RT. Reaction mixture was basified with K₂CO₃ solution and extracted with EtOAc. Organic layer was dried over Na₂SO₄ and distilled out to get crude product. The crude product was purified by preparative HPLC to get 90 mg of pure product. ¹H NMR (DMSO-d₆): 10.61 (s, 1H), 8.12-8.09 (m, 2H), 7.86 (d, J=8.4 Hz, 2H), 7.75 (s, 2H), 7.57 (d, J=8.0 Hz, 2H), 7.45 (t, 2H), 3.81 (s, 2H), 3.21 (q, J=6.8 Hz 2H), 1.91-1.88 (m, 2H), 1.54-1.51 (m, 2H), 1.06 (t, J=7.2 Hz, 3H). ESI-MS (m/z): 573.95 (M+H)⁺.

### Example 62

### Preparation of N-(3,5-dichloro-4-(1-(5-(4-(trifluoromethyl)phenyl)-1,2,4-oxadiazol-3-yl)cyclopropyl)phenyl)-2-(4-(ethylsulfonimidoyl)phenyl)acetamide

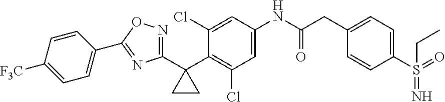 [SMILES: CCS(=N)(=O)c1ccc(CC(=O)Nc2cc(Cl)c(C3(c4noc(-c5ccc(C(F)(F)F)cc5)n4)CC3)c(Cl)c2)cc1]

Prepared using intermediate IV-3 and intermediate III-3 using similar procedure as described for example 61. ¹H NMR (DMSO-d₆): 10.62 (s, 1H), 8.26 (d, J=8.0 Hz, 2H), 7.98 (d, J=8.4 Hz, 2H), 7.87 (d, J=8.4 Hz, 2H), 7.75 (s, 2H), 7.58 (d, J=8.0 Hz, 2H), 3.82 (s, 2H), 3.21 (q, J=7.2 Hz 2H), 1.93-1.90 (m, 2H), 1.57-1.50 (m, 2H), 1.06 (t, J=7.2 Hz, 3H). ESI-MS (m/z): 623.00 (M+H)⁺.

### Example 63

### Preparation of 2-(4-(N-cyclopropylsulfamoyl)phenyl)-N-(3,5-dichloro-4-(1-(5-(4-fluorophenyl)-1,2,4-oxadiazol-3-yl)cyclopropyl)phenyl)acetamide

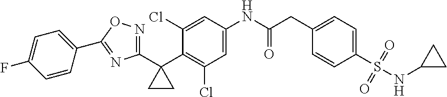 [SMILES: O=C(Cc1ccc(S(=O)(=O)NC2CC2)cc1)Nc1cc(Cl)c(C2(c3noc(-c4ccc(F)cc4)n3)CC2)c(Cl)c1]

Prepared using intermediate IV-4 and intermediate III-1. ¹H NMR (DMSO-d₆): 10.61 (s, 1H), 8.12-8.09 (m, 2H), 7.90 (bd, 1H), 7.78 (s, 2H), 7.76 (d, J=4.4 Hz, 2H), 7.56 (d, J=8.4 Hz, 2H), 7.45 (t, 2H), 3.81 (s, 2H), 2.08-2.05 (m, 1H), 1.91-1.88 (m, 2H), 1.54-1.51 (m, 2H), 0.49-0.39 (m, 4H). ESI-MS (m/z): 599.05 (M−H).

### Example 64

### Preparation of 2-(4-(N-cyclopropylsulfamoyl)phenyl)-N-(3,5-dichloro-4-(1-(5-(4-(trifluoromethyl)phenyl)-1,2,4-oxadiazol-3-yl)cyclopropyl)phenyl)acetamide

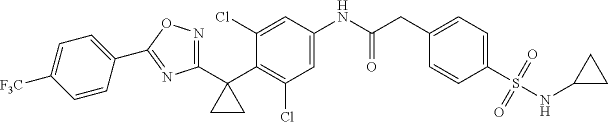 [SMILES: O=C(Cc1ccc(S(=O)(=O)NC2CC2)cc1)Nc1cc(Cl)c(C2(c3noc(-c4ccc(C(F)(F)F)cc4)n3)CC2)c(Cl)c1]

Prepared using intermediate IV-4 and intermediate III-3. ¹H NMR (DMSO-d₆): 10.62 (s, 1H), 8.26 (d, J=8 Hz, 2H), 7.99 (d, J=8.4 Hz, 2H), 7.90 (bd, 1H), 7.78 (s, 2H), 7.76 (d, 2H), 7.56 (d, J=8.4 Hz, 2H), 3.81 (s, 2H), 2.2-2.1 (m, 1H), 1.92 (bd, 2H), 1.56 (bd, 2H), 0.46-0.37 (m, 4H). ESI-MS (m/z): 649.05 (M−H).

### Example 65

### Preparation of 2-(4-(N-cyclopropylsulfamoyl)phenyl)-N-(3,5-dichloro-4-(1-(6-fluorobenzo[d]oxazol-2-yl)cyclopropyl)phenyl)acetamide

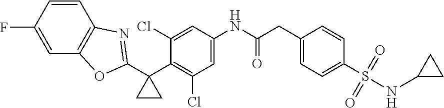 [SMILES: O=C(Cc1ccc(S(=O)(=O)NC2CC2)cc1)Nc1cc(Cl)c(C2(c3nc4ccc(F)cc4o3)CC2)c(Cl)c1]

Prepared using intermediate IV-4 and intermediate III-39. ¹H NMR (DMSO-d₆): 10.62 (s, 1H), 7.90 (d, J=2.4 Hz, 1H), 7.78 (d, 2H), 7.76 (s, 2H), 7.62-7.59 (m, 2H), 7.56 (d, J=8.4 Hz, 2H), 7.2-7.1 (m, 1H), 3.81 (s, 2H), 2.06-2.04 (m, 3H), 1.63-1.62 (m, 2H), 0.48 (m, 2H), 0.38 (m, 2H). ESI-MS (m/z): 574.50 (M+H)⁺.

### Example 66

### Preparation of N-(3,5-dichloro-4-(1-(5-(4-fluorophenyl)-1,2,4-oxadiazol-3-yl) cyclopropyl)phenyl)-2-(6-(ethylsulfonyl)pyridin-3-yl)acetamide

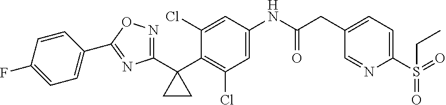 [SMILES: CCS(=O)(=O)c1ccc(CC(=O)Nc2cc(Cl)c(C3(c4noc(-c5ccc(F)cc5)n4)CC3)c(Cl)c2)cn1]

### Step 1: N-(3,5-dichloro-4-(1-(5-(4-fluorophenyl)-1,2,4-oxadiazol-3-yl)cyclopropyl) phenyl)-2-(6-(ethylthio)pyridin-3-yl)acetamide

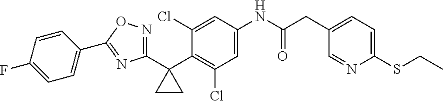 [SMILES: CCSc1ccc(CC(=O)Nc2cc(Cl)c(C3(c4noc(-c5ccc(F)cc5)n4)CC3)c(Cl)c2)cn1]

Prepared using intermediate V-1 and intermediate III-1 using similar procedure as described for example 1. ESI-MS (m/z): 543.05 (M+H)⁺.

### Step 2: N-(3,5-dichloro-4-(1-(5-(4-fluorophenyl)-1,2,4-oxadiazol-3-yl)cyclopropyl) phenyl)-2-(6-(ethylsulfonyl)pyridin-3-yl)acetamide

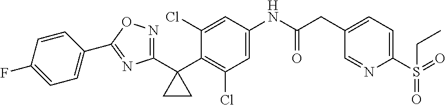 [SMILES: CCS(=O)(=O)c1ccc(CC(=O)Nc2cc(Cl)c(C3(c4noc(-c5ccc(F)cc5)n4)CC3)c(Cl)c2)cn1]

To a stirring solution of product of step 1 (60 mg, 0.110 mmol) in acetone (5 ml) and water (2 ml) was added oxone (0.204 g, 0.331 mmol) and stirred for 3 h at RT. The reaction mixture was diluted with EtOAc, washed with water and evaporated under reduced pressure to get title product as solid. ¹H NMR (DMSO-d₆): 10.66 (s, 1H), 8.73 (d, J=1.2 Hz, 1H), 8.12-8.08 (m, 4H), 7.74 (s, 2H), 7.45 (t, 2H), 3.93 (s, 2H), 3.44 (q, J=7.2 Hz, 2H), 1.90-1.89 (m, 2H), 1.54-1.53 (m, 2H), 1.13 (t, J=7.2 Hz, 3H). ESI-MS (m/z): 575.50 (M+H)⁺.

Using appropriate starting materials and suitable modifications of the process described in example 66, including suitable addition and/or deletion of steps as may be necessary, well within the scope of a person skilled in the art, the following compounds were prepared in an analogues manner.

### Example 67

### Preparation of N-(3,5-dichloro-4-(1-(5-(4-(trifluoromethyl)phenyl)-1,2,4-oxadiazol-3-yl)cyclopropyl)phenyl)-2-(6-(ethylsulfonyl)pyridin-3-yl)acetamide

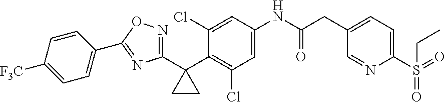 [SMILES: CCS(=O)(=O)c1ccc(CC(=O)Nc2cc(Cl)c(C3(c4noc(-c5ccc(C(F)(F)F)cc5)n4)CC3)c(Cl)c2)cn1]

Prepared using intermediate V-1 and intermediate III-3. ¹H NMR (DMSO-d₆): 10.67 (s, 1H), 8.74 (d, J=1.6 Hz, 1H), 8.26 (d, J=8.0 Hz, 2H), 8.09 (d, J=2.0 Hz, 1H), 8.05 (d, J=8.0 Hz, 1H), 7.99 (d, J=8.0 Hz, 2H), 7.75 (s, 2H), 3.93 (s, 2H), 3.46 (q, J=7.6 Hz, 2H), 1.93-1.90 (m, 2H), 1.58-1.56 (m, 2H), 1.13 (t, J=7.6 Hz, 3H). ESI-MS (m/z): 625.05 (M+H)⁺.

### Example 68

### Preparation of N-(3,5-dichloro-4-(1-(5-(3,4-difluorophenyl)-1,2,4-oxadiazol-3-yl)cyclopropyl)phenyl)-2-(6-(ethylsulfonyl)pyridin-3-yl)acetamide

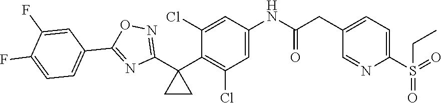 [SMILES: CCS(=O)(=O)c1ccc(CC(=O)Nc2cc(Cl)c(C3(c4noc(-c5ccc(F)c(F)c5)n4)CC3)c(Cl)c2)cn1]

Prepared using intermediate V-1 and intermediate III-5. ¹H NMR (DMSO-d₆): 10.66 (s, 1H), 8.73 (d, J=1.2 Hz, 1H), 8.12-8.08 (m, 2H), 8.05 (d, J=8.4 Hz, 1H), 7.93 (d, J=8.0 Hz, 1H), 7.74 (s, 2H), 7.72-7.65 (m, 1H), 3.93 (s, 2H), 3.46 (q, J=7.2 Hz, 2H), 1.91-1.88 (m, 2H), 1.56-1.53 (m, 2H), 1.13 (t, J=7.2 Hz, 3H). ESI-MS (m/z): 593.65 (M+H)⁺.

### Example 69

### Preparation of N-(3,5-dichloro-4-(1-(5-(2,4-difluorophenyl)-1,2,4-oxadiazol-3-yl) cyclopropyl)phenyl)-2-(6-(ethylsulfonyl)pyridin-3-yl)acetamide

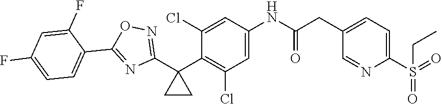 [SMILES: CCS(=O)(=O)c1ccc(CC(=O)Nc2cc(Cl)c(C3(c4noc(-c5ccc(F)cc5F)n4)CC3)c(Cl)c2)cn1]

Prepared using intermediate V-1 and intermediate III-7. ¹H NMR (DMSO-d₆): 10.65 (s, 1H), 8.73 (d, J=1.6 Hz, 1H), 8.15-8.10 (m, 1H), 8.09 (d, J=2.4 Hz, 1H), 8.05 (d, J=8.0 Hz, 1H), 7.74 (s, 2H), 7.62-7.56 (m, 1H), 7.34-7.33 (m, 1H), 3.93 (s, 2H), 3.46 (q, J=7.2 Hz, 2H), 1.90-1.87 (m, 2H), 1.56-1.53 (m, 2H), 1.13 (t, J=7.2 Hz, 3H). ESI-MS (m/z): 593.00 (M+H)⁺.

### Example 70

### Preparation of N-(3,5-dichloro-4-(1-(5-(3-chloro-4-fluorophenyl)-1,2,4-oxadiazol-3-yl)cyclopropyl)phenyl)-2-(6-(ethylsulfonyl)pyridin-3-yl)acetamide

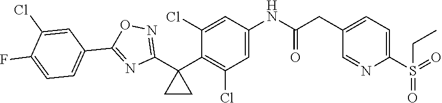 [SMILES: CCS(=O)(=O)c1ccc(CC(=O)Nc2cc(Cl)c(C3(c4noc(-c5ccc(F)c(Cl)c5)n4)CC3)c(Cl)c2)cn1]

Prepared using intermediate V-1 and intermediate III-10. ¹H NMR (DMSO-d₆): 10.67 (s, 1H), 8.73 (d, J=1.6 Hz, 1H), 8.24 (dd, J=2.0 & 6.8 Hz, 1H), 8.11-8.03 (m, 3H), 7.74 (s, 2H), 7.68-7.64 (m, 1H), 3.93 (s, 2H), 3.46 (q, J=7.6 Hz, 2H), 1.92-1.89 (m, 2H), 1.56-1.53 (m, 2H), 1.12 (t, J=7.2 Hz, 3H). ESI-MS (m/z): 609.00 (M+H)⁺.

### Example 71

### Preparation of N-(3,5-dichloro-4-(1-(5-(3,4-dichlorophenyl)-1,2,4-oxadiazol-3-yl)cyclopropyl)phenyl)-2-(6-(ethylsulfonyl)pyridin-3-yl)acetamide

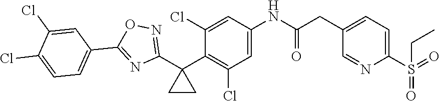 [SMILES: CCS(=O)(=O)c1ccc(CC(=O)Nc2cc(Cl)c(C3(c4noc(-c5ccc(Cl)c(Cl)c5)n4)CC3)c(Cl)c2)cn1]

Prepared using intermediate V-1 and intermediate III-11. ¹H NMR (DMSO-d₆): 10.67 (s, 1H), 8.73 (bs, 1H), 8.23 (d, J=2.0 Hz, 1H), 8.11-7.99 (m, 3H), 7.89 (d, J=8.4 Hz, 1H), 7.74 (s, 2H), 3.93 (s, 2H), 3.46 (q, J=7.6 Hz, 2H), 1.92-1.89 (m, 2H), 1.56-1.53 (m, 2H), 1.12 (t, J=7.2 Hz, 3H). ESI-MS (m/z): 626.95 (M+H)⁺.

### Example 72

### Preparation of N-(3,5-dichloro-4-(1-(5-(4-chlorophenyl)-1,2,4-oxadiazol-3-yl) cyclopropyl)phenyl)-2-(6-(ethylsulfonyl)pyridin-3-yl)acetamide

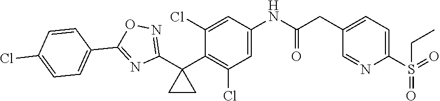 [SMILES: CCS(=O)(=O)c1ccc(CC(=O)Nc2cc(Cl)c(C3(c4noc(-c5ccc(Cl)cc5)n4)CC3)c(Cl)c2)cn1]

Prepared using intermediate V-1 and intermediate III-13. ¹H NMR (CDCl₃): 8.74 (s, 1H), 8.63 (d, J=1.6 Hz, 1H), 8.08-8.04 (m, 3H), 7.97 (dd, J=2.0 & 8 Hz, 1H), 7.56-7.52 (m, 2H), 7.49 (s, 2H), 3.76 (s, 2H), 3.44 (q, J=7.6 Hz, 2H), 2.09-2.06 (m, 2H), 1.60-1.55 (m, 2H), 1.32 (t, J=7.6 Hz, 3H). ESI-MS (m/z): 593.00 (M+H)⁺.

### Example 73

### Preparation of N-(3,5-dichloro-4-(1-(5-(4-cyanophenyl)-1,2,4-oxadiazol-3-yl) cyclopropyl)phenyl)-2-(6-(ethylsulfonyl)pyridin-3-yl)acetamide

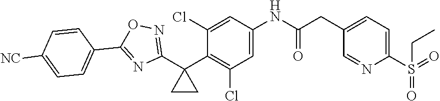 [SMILES: CCS(=O)(=O)c1ccc(CC(=O)Nc2cc(Cl)c(C3(c4noc(-c5ccc(C#N)cc5)n4)CC3)c(Cl)c2)cn1]

Prepared using intermediate V-1 and intermediate III-16. ¹H NMR (DMSO-d₆): 10.67 (s, 1H), 8.73 (d, J=1.6 Hz, 1H), 8.21 (dd, J=2.0 & 6.8 Hz, 2H), 8.11-8.03 (m, 4H), 7.74 (s, 2H), 3.93 (s, 2H), 3.46 (q, J=7.2 Hz, 2H), 1.93-1.90 (m, 2H), 1.58-1.55 (m, 2H), 1.12 (t, J=7.2 Hz, 3H). ESI-MS (m/z): 582.05 (M+H)⁺.

### Example 74

### Preparation of N-(3,5-dichloro-4-(1-(6-fluorobenzo[d]oxazol-2-yl) cyclopropyl) phenyl)-2-(6-(ethylsulfonyl)pyridin-3-yl)acetamide

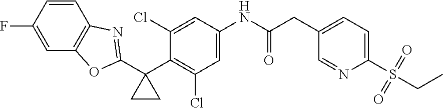 [SMILES: CCS(=O)(=O)c1ccc(CC(=O)Nc2cc(Cl)c(C3(c4nc5ccc(F)cc5o4)CC3)c(Cl)c2)cn1]

Prepared using intermediate V-1 and intermediate III-39. ¹H NMR (CDCl₃): 9.62 (s, 1H), 8.53 (d, J=2.0 Hz, 1H), 8.07 (d, J=8.0 Hz, 1H), 7.91 (dd, J=2.0 & 8.0 Hz, 1H), 7.56 (s, 2H), 7.48 (dd, J=4.8 & 8.8 Hz, 1H), 7.28-7.26 (m, 1H), 7.12-7.08 (m, 1H), 3.68 (s, 2H), 3.43 (q, J=7.6 Hz, 2H), 2.23-2.20 (m, 2H), 1.70-1.67 (m, 2H), 1.32 (t, J=7.6 Hz, 3H). ESI-MS (m/z): 548.00 (M+H)⁺.

### Example 75

### Preparation of 2-(6-(ethylsulfonyl)pyridin-3-yl)-N-(4-(1-(5-(4-fluorophenyl)-1,2,4-oxadiazol-3-yl)cyclopropyl)-3,5-dimethylphenyl)acetamide

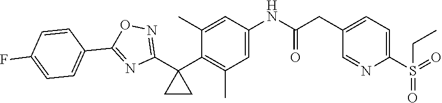 [SMILES: CCS(=O)(=O)c1ccc(CC(=O)Nc2cc(C)c(C3(c4noc(-c5ccc(F)cc5)n4)CC3)c(C)c2)cn1]

Prepared using intermediate V-1 and intermediate III-36. ¹H NMR (DMSO-d₆): 10.19 (s, 1H), 8.73 (d, J=1.2 Hz, 1H), 8.12-8.07 (m, 3H), 8.04 (d, J=8.0 Hz, 1H), 7.46-7.42 (m, 2H), 7.27 (s, 2H), 3.86 (s, 2H), 3.46 (q, J=7.2 Hz, 2H), 2.27 (s, 6H), 1.76-1.73 (m, 2H), 1.33-1.30 (m, 2H), 1.12 (t, J=7.2 Hz, 3H). ESI-MS (m/z): 535.15 (M+H)⁺.

### Example 76

### Preparation of 2-(6-(ethylsulfonyl)pyridin-3-yl)-N-(4-(1-(6-fluorobenzo[d]oxazol-2-yl)cyclopropyl)-3,5-dimethylphenyl)acetamide

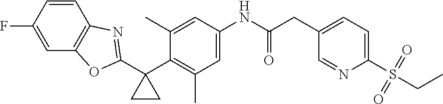 [SMILES: CCS(=O)(=O)c1ccc(CC(=O)Nc2cc(C)c(C3(c4nc5ccc(F)cc5o4)CC3)c(C)c2)cn1]

Prepared using intermediate V-1 and intermediate III-40. ¹H NMR (DMSO-d₆): 10.22 (s, 1H), 8.73 (s, 1H), 8.10-8.03 (m, 2H), 7.60 (dd, J=3.6 & 8.4 Hz, 2H), 7.29 (s, 2H), 7.18-7.16 (m, 1H), 3.87 (s, 2H), 3.46 (q, J=7.6 Hz, 2H), 2.28 (s, 6H), 1.92-1.89 (m, 2H), 1.43-1.40 (m, 2H), 1.13 (t, J=7.2 Hz, 3H). ESI-MS (m/z): 508.15 (M+H)⁺.

### Example 77

### Preparation of N-(3,5-dichloro-4-(1-(5-(4-fluorophenyl)-1,2,4-oxadiazol-3-yl) cyclopropyl)phenyl)-2-(5-(ethylsulfonyl)pyrazin-2-yl)acetamide

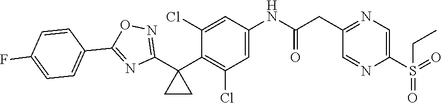 [SMILES: CCS(=O)(=O)c1cnc(CC(=O)Nc2cc(Cl)c(C3(c4noc(-c5ccc(F)cc5)n4)CC3)c(Cl)c2)cn1]

Prepared using intermediate V-2 and intermediate III-1. ¹H NMR (DMSO-d₆): 10.72 (s, 1H), 9.17 (d, J=1.6 Hz, 1H), 8.93 (d, J=1.2 Hz, 1H), 8.12-8.09 (m, 2H), 7.74 (s, 2H), 7.47-7.42 (m, 2H), 4.17 (s, 2H), 3.51 (q, J=7.6 Hz, 2H), 1.90 (bd, 2H), 1.54 (bd, 2H), 1.18 (t, J=7.2 Hz, 3H). ESI-MS (m/z): 576.95 (M+H)⁺.

### Example 78

### Preparation of N-(3,5-dichloro-4-(1-(6-fluorobenzo[d]oxazol-2-yl)cyclopropyl) phenyl)-2-(5-(ethylsulfonyl)pyrazin-2-yl)acetamide

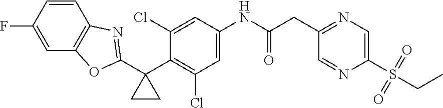 [SMILES: CCS(=O)(=O)c1cnc(CC(=O)Nc2cc(Cl)c(C3(c4nc5ccc(F)cc5o4)CC3)c(Cl)c2)cn1]

Prepared using intermediate V-2 and intermediate III-39. ¹H NMR (CDCl₃): 9.61 (s, 1H), 9.23 (d, J=1.2 Hz, 1H), 8.78 (d, J=1.2 Hz, 1H), 7.60 (s, 2H), 7.56-7.53 (m, 1H), 7.21 (dd, J=2.4 & 8.0 Hz, 1H), 7.08-7.03 (m, 1H), 4.00 (s, 2H), 3.47 (q, J=7.2 Hz, 2H), 2.19-2.16 (m, 2H), 1.67-1.63 (m, 2H), 1.37 (t, J=7.6 Hz, 3H). ESI-MS (m/z): 549.55 (M+H)⁺.

### Example 79

### Preparation of N-(3,5-dichloro-4-(1-(5-(4-fluorophenyl)-1,3,4-oxadiazol-2-yl) cyclopropyl)phenyl)-2-(4-(ethylsulfonyl)phenyl)acetamide

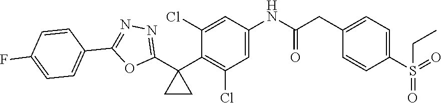 [SMILES: CCS(=O)(=O)c1ccc(CC(=O)Nc2cc(Cl)c(C3(c4nnc(-c5ccc(F)cc5)o4)CC3)c(Cl)c2)cc1]

Prepared using intermediate IV-1 and intermediate III-44. ¹H NMR (DMSO-d₆): 10.65 (s, 1H), 7.99-7.95 (m, 2H), 7.86 (d, J=8.4 Hz, 2H), 7.77 (s, 2H), 7.61 (d, J=8.4 Hz, 2H), 7.43-7.39 (m, 2H), 3.84 (s, 2H), 3.30 (q, J=7.2 Hz, 2H), 2.06-2.03 (m, 2H), 1.62-1.59 (m, 2H), 1.09 (t, J=7.6 Hz, 3H). ESI-MS (m/z): 574.05 (M+H)⁺.

### Example 80

### Preparation of N-(3,5-dichloro-4-(1-(5-(4-fluorophenyl)-1,3,4-oxadiazol-2-yl) cyclopropyl)phenyl)-2-(5-(ethylsulfonyl)pyridin-2-yl)acetamide

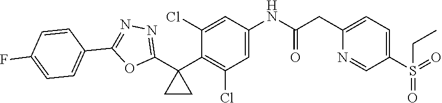 [SMILES: CCS(=O)(=O)c1ccc(CC(=O)Nc2cc(Cl)c(C3(c4nnc(-c5ccc(F)cc5)o4)CC3)c(Cl)c2)nc1]

Prepared using intermediate IV-2 and intermediate III-44. ¹H NMR (DMSO-d₆): 10.71 (s, 1H), 8.96 (d, J=2.4 Hz, 1H), 8.28 (dd, J=2.0 & 8.0 Hz, 1H), 7.99-7.95 (m, 2H), 7.78 (s, 2H), 7.72 (d, J=8.4 Hz, 1H), 7.41 (t, 2H), 4.05 (s, 2H), 3.41 (q, J=7.2 Hz, 2H), 2.06-2.03 (m, 2H), 1.63-1.60 (m, 2H), 1.13 (t, J=7.2 Hz, 3H). ESI-MS (m/z): 575.45 (M+H)⁺.

The following compounds can be prepared by procedure similar to those described above with appropriate variations of reactions, reaction conditions, reagents and quantities of reagents which are within the scope of person skilled in the art.

### Example 81

### Preparation of N-(3,5-dichloro-4-(1-(5-(4-fluorophenyl)-1,2,4-oxadiazol-3-yl)cyclopropyl)phenyl)-2-(4-(methylsulfonyl)phenyl)acetamide

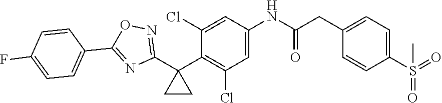 [SMILES: CS(=O)(=O)c1ccc(CC(=O)Nc2cc(Cl)c(C3(c4noc(-c5ccc(F)cc5)n4)CC3)c(Cl)c2)cc1]

### Preparation of N-(3,5-dichloro-4-(1-(5-(4-chlorophenyl)-1,2,4-oxadiazol-3-yl)cyclopropyl)phenyl)-2-(4-(methylsulfonyl)phenyl)acetamide

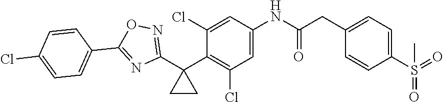 [SMILES: CS(=O)(=O)c1ccc(CC(=O)Nc2cc(Cl)c(C3(c4noc(-c5ccc(Cl)cc5)n4)CC3)c(Cl)c2)cc1]

### Example 83

### Preparation of N-(3,5-dichloro-4-(1-(5-(4-(trifluoromethyl)phenyl)-1,2,4-oxadiazol-3-yl)cyclopropyl)phenyl)-2-(4-(methylsulfonyl)phenyl)acetamide

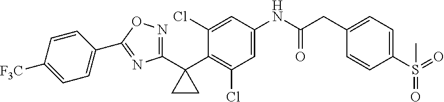 [SMILES: CS(=O)(=O)c1ccc(CC(=O)Nc2cc(Cl)c(C3(c4noc(-c5ccc(C(F)(F)F)cc5)n4)CC3)c(Cl)c2)cc1]

### Preparation of N-(3,5-dichloro-4-(1-(5-(4-cyanophenyl)-1,2,4-oxadiazol-3-yl) cyclopropyl)phenyl)-2-(4-(methylsulfonyl)phenyl)acetamide

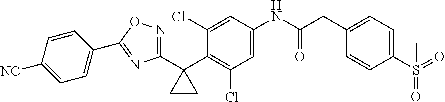 [SMILES: CS(=O)(=O)c1ccc(CC(=O)Nc2cc(Cl)c(C3(c4noc(-c5ccc(C#N)cc5)n4)CC3)c(Cl)c2)cc1]

### Example 85

### Preparation of N-(3,5-dichloro-4-(1-(5-(3-chloro-4-fluorophenyl)-1,2,4-oxadiazol-3-yl)cyclopropyl)phenyl)-2-(4-(methylsulfonyl)phenyl)acetamide

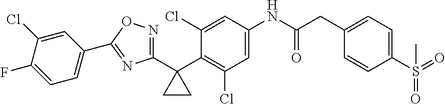 [SMILES: CS(=O)(=O)c1ccc(CC(=O)Nc2cc(Cl)c(C3(c4noc(-c5ccc(F)c(Cl)c5)n4)CC3)c(Cl)c2)cc1]

### Example 86

### Preparation of N-(3,5-dichloro-4-(1-(6-fluorobenzo[d]oxazol-2-yl) cyclopropyl) phenyl)-2-(4-(methylsulfonyl)phenyl)acetamide

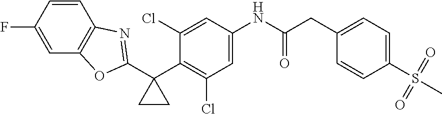 [SMILES: CS(=O)(=O)c1ccc(CC(=O)Nc2cc(Cl)c(C3(c4nc5ccc(F)cc5o4)CC3)c(Cl)c2)cc1]

### Preparation of N-(3,5-dichloro-4-(1-(3-(4-chlorophenyl)-1,2,4-oxadiazol-5-yl) cyclopropyl)phenyl)-2-(4-(ethylsulfonyl)phenyl)acetamide

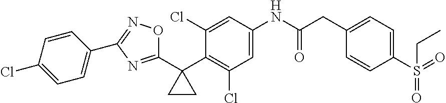 [SMILES: CCS(=O)(=O)c1ccc(CC(=O)Nc2cc(Cl)c(C3(c4nc(-c5ccc(Cl)cc5)no4)CC3)c(Cl)c2)cc1]

### Example 88

### Preparation of N-(3,5-dichloro-4-(1-(3-(4-(trifluoromethyl)phenyl)-1,2,4-oxadiazol-5-yl)cyclopropyl)phenyl)-2-(4-(ethylsulfonyl)phenyl)acetamide

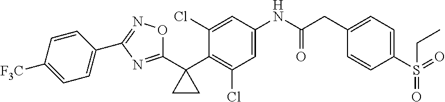 [SMILES: CCS(=O)(=O)c1ccc(CC(=O)Nc2cc(Cl)c(C3(c4nc(-c5ccc(C(F)(F)F)cc5)no4)CC3)c(Cl)c2)cc1]

### Example 89

### Preparation of N-(3,5-dichloro-4-(1-(3-(4-cyanophenyl)-1,2,4-oxadiazol-5-yl) cyclopropyl)phenyl)-2-(4-(ethylsulfonyl)phenyl)acetamide

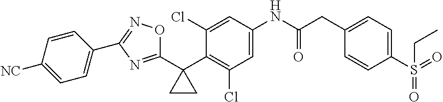 [SMILES: CCS(=O)(=O)c1ccc(CC(=O)Nc2cc(Cl)c(C3(c4nc(-c5ccc(C#N)cc5)no4)CC3)c(Cl)c2)cc1]

### Example 90

### Preparation of N-(3,5-dichloro-4-(1-(3-(3,4-difluorophenyl)-1,2,4-oxadiazol-5-yl) cyclopropyl)phenyl)-2-(4-(ethylsulfonyl)phenyl)acetamide

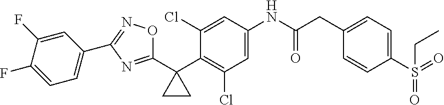 [SMILES: CCS(=O)(=O)c1ccc(CC(=O)Nc2cc(Cl)c(C3(c4nc(-c5ccc(F)c(F)c5)no4)CC3)c(Cl)c2)cc1]

### Example 91

### Preparation of N-(3,5-dichloro-4-(1-(3-(3-chloro-4-fluorophenyl)-1,2,4-oxadiazol-5-yl)cyclopropyl)phenyl)-2-(4-(ethylsulfonyl)phenyl)acetamide

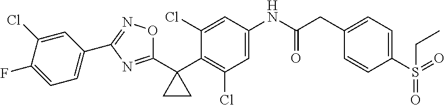 [SMILES: CCS(=O)(=O)c1ccc(CC(=O)Nc2cc(Cl)c(C3(c4nc(-c5ccc(F)c(Cl)c5)no4)CC3)c(Cl)c2)cc1]

### Example 92

### Preparation of N-(3,5-dichloro-4-(1-(3-(2,4-difluorophenyl)-1,2,4-oxadiazol-5-yl) cyclopropyl)phenyl)-2-(4-(ethylsulfonyl)phenyl)acetamide

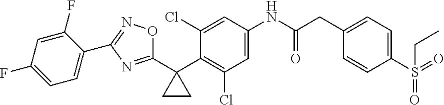 [SMILES: CCS(=O)(=O)c1ccc(CC(=O)Nc2cc(Cl)c(C3(c4nc(-c5ccc(F)cc5F)no4)CC3)c(Cl)c2)cc1]

### Example 93

### Preparation of N-(3,5-dichloro-4-(1-(3-(p-tolyl)-1,2,4-oxadiazol-5-yl) cyclopropyl) phenyl)-2-(4-(ethylsulfonyl)phenyl)acetamide

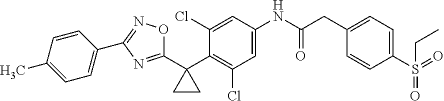 [SMILES: CCS(=O)(=O)c1ccc(CC(=O)Nc2cc(Cl)c(C3(c4nc(-c5ccc(C)cc5)no4)CC3)c(Cl)c2)cc1]

### Example 94

### Preparation of N-(3,5-dichloro-4-(1-(3-phenyl-1,2,4-oxadiazol-5-yl)cyclopropyl) phenyl)-2-(4-(ethylsulfonyl)phenyl)acetamide

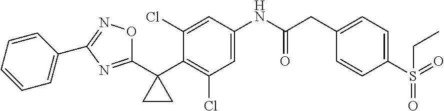 [SMILES: CCS(=O)(=O)c1ccc(CC(=O)Nc2cc(Cl)c(C3(c4nc(-c5ccccc5)no4)CC3)c(Cl)c2)cc1]

### Example 95

### Preparation of N-(3,5-dichloro-4-(1-(3-cyclopropyl-1,2,4-oxadiazol-5-yl) cyclopropyl)phenyl)-2-(4-(ethylsulfonyl)phenyl)acetamide

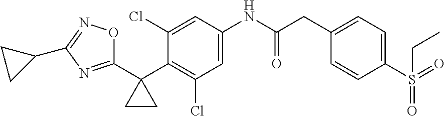 [SMILES: CCS(=O)(=O)c1ccc(CC(=O)Nc2cc(Cl)c(C3(c4nc(C5CC5)no4)CC3)c(Cl)c2)cc1]

### Example 96

### Preparation of N-(3,5-dichloro-4-(1-(3-(3,3-difluorocyclobutyl)-1,2,4-oxadiazol-5-yl) cyclopropyl)phenyl)-2-(4-(ethylsulfonyl)phenyl)acetamide

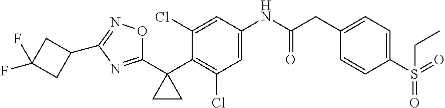 [SMILES: CCS(=O)(=O)c1ccc(CC(=O)Nc2cc(Cl)c(C3(c4nc(C5CC(F)(F)C5)no4)CC3)c(Cl)c2)cc1]

### Example 97

### Preparation of N-(3,5-dichloro-4-(1-(3-(2-fluoro-4-methoxyphenyl)-1,2,4-oxadiazol-5-yl)cyclopropyl)phenyl)-2-(4-(ethylsulfonyl)phenyl)acetamide

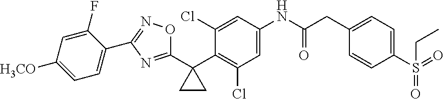 [SMILES: CCS(=O)(=O)c1ccc(CC(=O)Nc2cc(Cl)c(C3(c4nc(-c5ccc(OC)cc5F)no4)CC3)c(Cl)c2)cc1]

### Example 98

### Preparation of 2-(4-(ethylsulfonyl)phenyl)-N-(4-(1-(3-(4-fluorophenyl)-1,2,4-oxadiazol-5-yl)cyclopropyl)-3,5-dimethylphenyl)acetamide

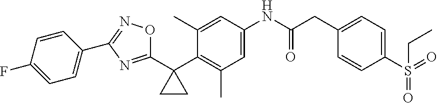 [SMILES: CCS(=O)(=O)c1ccc(CC(=O)Nc2cc(C)c(C3(c4nc(-c5ccc(F)cc5)no4)CC3)c(C)c2)cc1]

### Example 99

### Preparation of 2-(4-(ethylsulfonyl)phenyl)-N-(4-(1-(5-(4-fluorophenyl)oxazol-2-yl)cyclopropyl)-3,5-dimethylphenyl)acetamide

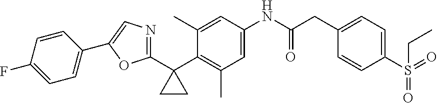 [SMILES: CCS(=O)(=O)c1ccc(CC(=O)Nc2cc(C)c(C3(c4ncc(-c5ccc(F)cc5)o4)CC3)c(C)c2)cc1]

### Example 100

### Preparation of N-(3,5-dichloro-4-(1-(5-(4-chlorophenyl)-1,2,4-oxadiazol-3-yl) cyclopropyl)phenyl)-2-(4-(ethylsulfonimidoyl)phenyl)acetamide

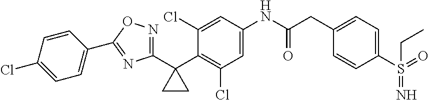 [SMILES: CCS(=N)(=O)c1ccc(CC(=O)Nc2cc(Cl)c(C3(c4noc(-c5ccc(Cl)cc5)n4)CC3)c(Cl)c2)cc1]

### Example 101

### Preparation of N-(3,5-dichloro-4-(1-(6-fluorobenzo[d]oxazol-2-yl) cyclopropyl) phenyl)-2-(4-(ethylsulfonimidoyl)phenyl)acetamide

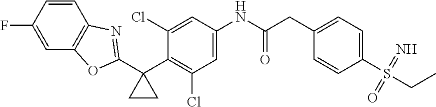 [SMILES: CCS(=N)(=O)c1ccc(CC(=O)Nc2cc(Cl)c(C3(c4nc5ccc(F)cc5o4)CC3)c(Cl)c2)cc1]

### Example 102

### Preparation of N-(3,5-dichloro-4-(1-(6-chlorobenzo[d]oxazol-2-yl) cyclopropyl) phenyl)-2-(4-(ethylsulfonimidoyl)phenyl)acetamide

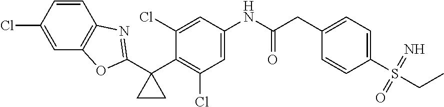 [SMILES: CCS(=N)(=O)c1ccc(CC(=O)Nc2cc(Cl)c(C3(c4nc5ccc(Cl)cc5o4)CC3)c(Cl)c2)cc1]

### Example 103

### Preparation of N-(4-(1-(benzo[d]oxazol-2-yl)cyclopropyl)-3,5-dichlorophenyl)-2-(4-(ethylsulfonyl)phenyl)acetamide

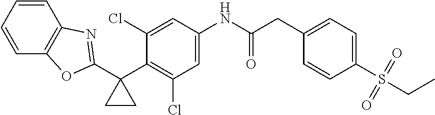 [SMILES: CCS(=O)(=O)c1ccc(CC(=O)Nc2cc(Cl)c(C3(c4nc5ccccc5o4)CC3)c(Cl)c2)cc1]

### Example 104

### Preparation of N-(3,5-dichloro-4-(1-(6-chlorobenzo[d]oxazol-2-yl)cyclopropyl) phenyl)-2-(4-(ethylsulfonyl)phenyl)acetamide

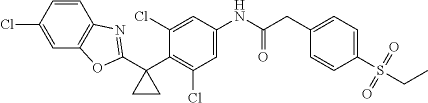 [SMILES: CCS(=O)(=O)c1ccc(CC(=O)Nc2cc(Cl)c(C3(c4nc5ccc(Cl)cc5o4)CC3)c(Cl)c2)cc1]

### Example 105

### Preparation of N-(3,5-dichloro-4-(1-(6-(trifluoromethyl)benzo[d]oxazol-2-yl)cyclopropyl)phenyl)-2-(4-(ethylsulfonyl)phenyl)acetamide

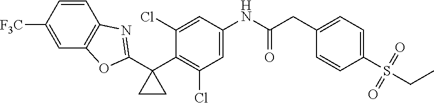 [SMILES: CCS(=O)(=O)c1ccc(CC(=O)Nc2cc(Cl)c(C3(c4nc5ccc(C(F)(F)F)cc5o4)CC3)c(Cl)c2)cc1]

### Example 106

### Preparation of N-(4-(1-(benzo[d]oxazol-2-yl)cyclopropyl)-3,5-dichlorophenyl)-2-(5-(ethylsulfonyl)pyridin-2-yl)acetamide

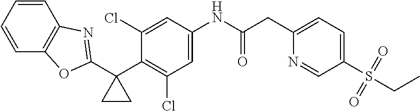 [SMILES: CCS(=O)(=O)c1ccc(CC(=O)Nc2cc(Cl)c(C3(c4nc5ccccc5o4)CC3)c(Cl)c2)nc1]

### Example 107

### Preparation of N-(3,5-dichloro-4-(1-(6-chlorobenzo[d]oxazol-2-yl)cyclopropyl) phenyl)-2-(5-(ethylsulfonyl)pyridin-2-yl)acetamide

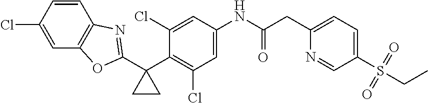 [SMILES: CCS(=O)(=O)c1ccc(CC(=O)Nc2cc(Cl)c(C3(c4nc5ccc(Cl)cc5o4)CC3)c(Cl)c2)nc1]

### Example 108

### Preparation of N-(3,5-dichloro-4-(1-(6-(trifluoromethyl)benzo[d]oxazol-2-yl)cyclopropyl)phenyl)-2-(5-(ethylsulfonyl)pyridin-2-yl)acetamide

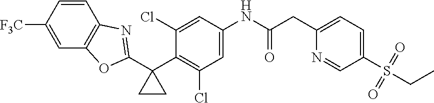 [SMILES: CCS(=O)(=O)c1ccc(CC(=O)Nc2cc(Cl)c(C3(c4nc5ccc(C(F)(F)F)cc5o4)CC3)c(Cl)c2)nc1]

### Example 109

### Preparation of N-(4-(1-(6-chlorobenzo[d]oxazol-2-yl)cyclopropyl)-3,5-dimethylphenyl)-2-(5-(ethylsulfonyl)pyridin-2-yl)acetamide

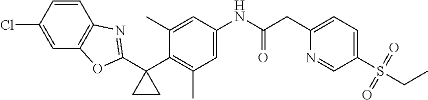 [SMILES: CCS(=O)(=O)c1ccc(CC(=O)Nc2cc(C)c(C3(c4nc5ccc(Cl)cc5o4)CC3)c(C)c2)nc1]

### Example 110

### Preparation of N-(3,5-dichloro-4-(1-(5-(4-chlorophenyl)-1,3,4-oxadiazol-2-yl)cyclopropyl)phenyl)-2-(4-(ethylsulfonyl)phenyl)acetamide

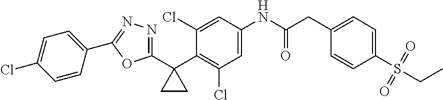 [SMILES: CCS(=O)(=O)c1ccc(CC(=O)Nc2cc(Cl)c(C3(c4nnc(-c5ccc(Cl)cc5)o4)CC3)c(Cl)c2)cc1]

### Example 111

### Preparation of N-(3,5-dichloro-4-(1-(5-(4-(trifluoromethyl)phenyl)-1,3,4-oxadiazol-2-yl)cyclopropyl)phenyl)-2-(4-(ethylsulfonyl)phenyl)acetamide

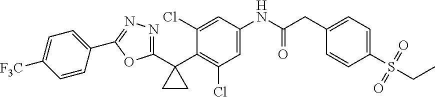 [SMILES: CCS(=O)(=O)c1ccc(CC(=O)Nc2cc(Cl)c(C3(c4nnc(-c5ccc(C(F)(F)F)cc5)o4)CC3)c(Cl)c2)cc1]

### Example 112

### Preparation of N-(4-(1-(5-(4-chlorophenyl)-1,3,4-oxadiazol-2-yl)cyclopropyl)-3,5-dimethylphenyl)-2-(4-(ethylsulfonyl)phenyl)acetamide

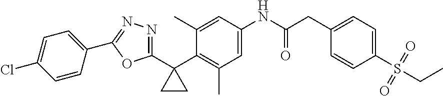 [SMILES: CCS(=O)(=O)c1ccc(CC(=O)Nc2cc(C)c(C3(c4nnc(-c5ccc(Cl)cc5)o4)CC3)c(C)c2)cc1]

### Example 113

### Preparation of 2-(4-(N-cyclopropylsulfamoyl)phenyl)-N-(3,5-dichloro-4-(1-(5-(4-chiorophenyl)-1,2,4-oxadiazol-3-yl)cyclopropyl)phenyl)acetamide

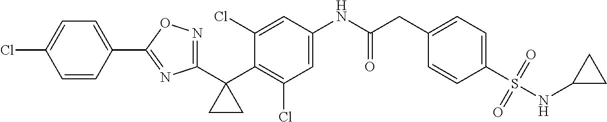 [SMILES: O=C(Cc1ccc(S(=O)(=O)NC2CC2)cc1)Nc1cc(Cl)c(C2(c3noc(-c4ccc(Cl)cc4)n3)CC2)c(Cl)c1]

### Example 114

### Preparation of 2-(4-(N-cyclopropylsulfamoyl)phenyl)-N-(3,5-dichloro-4-(1-(6-chlorobenzo[d]oxazol-2-yl)cyclopropyl)phenyl)acetamide

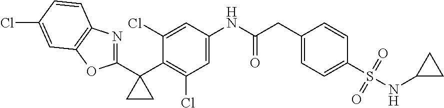 [SMILES: O=C(Cc1ccc(S(=O)(=O)NC2CC2)cc1)Nc1cc(Cl)c(C2(c3nc4ccc(Cl)cc4o3)CC2)c(Cl)c1]

### Example 115

### Preparation of N-(3,5-dichloro-4-(1-(5-(4-cyclopropylphenyl)-1,2,4-oxadiazol-3-yl)cyclopropyl)phenyl)-2-(4-(ethylsulfonyl)phenyl)acetamide

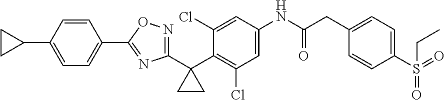 [SMILES: CCS(=O)(=O)c1ccc(CC(=O)Nc2cc(Cl)c(C3(c4noc(-c5ccc(C6CC6)cc5)n4)CC3)c(Cl)c2)cc1]

### Example 116

### Preparation of N-(4-(1-(5-(4-chlorophenyl)-1,2,4-oxadiazol-3-yl)cyclopropyl)-3,5-difluorophenyl)-2-(4-(ethylsulfonyl)phenyl)acetamide

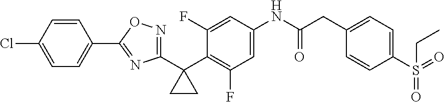 [SMILES: CCS(=O)(=O)c1ccc(CC(=O)Nc2cc(F)c(C3(c4noc(-c5ccc(Cl)cc5)n4)CC3)c(F)c2)cc1]

### Example 117

### Preparation of N-(3,5-difluoro-4-(1-(5-(4-(trifluoromethyl)phenyl)-1,2,4-oxadiazol-3-yl)cyclopropyl)phenyl)-2-(4-(ethylsulfonyl)phenyl)acetamide

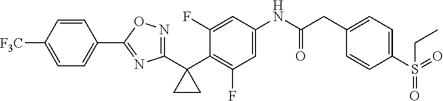 [SMILES: CCS(=O)(=O)c1ccc(CC(=O)Nc2cc(F)c(C3(c4noc(-c5ccc(C(F)(F)F)cc5)n4)CC3)c(F)c2)cc1]

### Example 118

### Preparation of N-(4-(1-(5-(4-cyanophenyl)-1,2,4-oxadiazol-3-yl)cyclopropyl)-3,5-difluorophenyl)-2-(4-(ethylsulfonyl)phenyl)acetamide

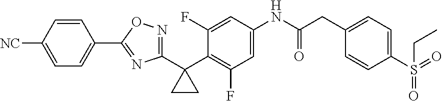 [SMILES: CCS(=O)(=O)c1ccc(CC(=O)Nc2cc(F)c(C3(c4noc(-c5ccc(C#N)cc5)n4)CC3)c(F)c2)cc1]

### Example 119

### Preparation of N-(4-(1-(5-(4-chlorophenyl)-1,2,4-oxadiazol-3-yl)cyclopropyl)-3,5-dimethylphenyl)-2-(4-(ethylsulfonyl)phenyl)acetamide

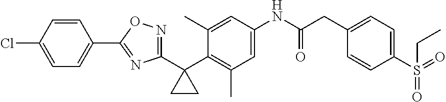 [SMILES: CCS(=O)(=O)c1ccc(CC(=O)Nc2cc(C)c(C3(c4noc(-c5ccc(Cl)cc5)n4)CC3)c(C)c2)cc1]

### Example 120

### Preparation of N-(3,5-dimethyl-4-(1-(5-(4-(trifluoromethyl)phenyl)-1,2,4-oxadiazol-3-yl)cyclopropyl)phenyl)-2-(4-(ethylsulfonyl)phenyl)acetamide

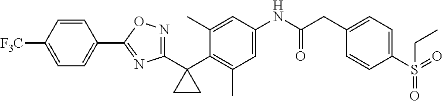 [SMILES: CCS(=O)(=O)c1ccc(CC(=O)Nc2cc(C)c(C3(c4noc(-c5ccc(C(F)(F)F)cc5)n4)CC3)c(C)c2)cc1]

### Example 121

### Preparation of N-(4-(1-(5-(4-cyanophenyl)-1,2,4-oxadiazol-3-yl)cyclopropyl)-3,5-dimethylphenyl)-2-(4-(ethylsulfonyl)phenyl)acetamide

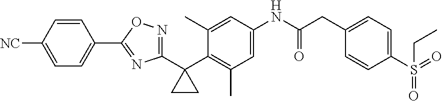 [SMILES: CCS(=O)(=O)c1ccc(CC(=O)Nc2cc(C)c(C3(c4noc(-c5ccc(C#N)cc5)n4)CC3)c(C)c2)cc1]

### In-Vitro Activity:

### 5×RORE Based Luciferase Assay:

Human RORγt (hRORγt) inhibitors were screened in RORE (RORγt Response Element) based Luciferase assay by transient transfection of 5×RORE (5 tandem repeats of RORγt Response Element) and full length human RORγt together in COS-7 cells. COS-7 cells were maintained as monolayer in complete DMEM (High Glucose) medium in presence of 2 mM Glutamin and 1× Sodium Pyruvate. Day before transfection, 15000 cells were seeded in 96 well cell culture plate in 100 μl antibiotic free medium and incubated at 37° C. in 5% CO₂ containing humidified chamber O/N. Prior to transfection, cells were fed with fresh complete growth medium and incubated until the addition of transfection complex. Transfection complex for the required numbers of wells were prepared from pGL2-promoter-5×RORE-Luc plasmid, pcDNA3.1 (+)-hRORγt expression plasmid, pβ-GAL plasmid (transfection control), and Lipofectamine 3000 (Invitrogen). 50 μl of transfection complex were added in 100 μl of complete medium to respective wells, mixed gently and plate was incubated for 5-6 h at 37° C. in 5% CO₂ containing humidified chamber. After 5 h of transfection, content of the wells were aspirated and cells were treated with increasing concentration of RORγt inverse agonist in medium devoid of serum with a final DMSO concentration of 0.2% for 18-20 h at 37° C. in 5% CO₂ containing humidified chamber. Next day, cells were lysed and the lysates were assayed for luciferase and β-GAL activity using Promega's luciferase assay system and an in-house made β-GAL assay buffer respectively. Luciferase signals were normalized with β-GAL and % activity was determined with respect to that of 10 nM control ligand.

RORγt inhibitory activity displayed by compounds of the present invention in the form of % inhibition at 100 nM concentration was found to be very good. IC₅₀ of selected compounds were then determined by nonlinear regression analysis of % activity, plotted against compound concentration (Table 4).

### Human IL-17 Inhibition Assay:

Peripheral blood mononuclear cells (PBMCs) were isolated from healthy volunteers and subjected to T cell polarization assay. Two million PBMCs were placed on anti-CD3 (Biolegend, US) coated 96-well plates and 1 μg/mL of anti-CD28 (Biolegend, US) was added along with RORγt inhibitors or the vehicle control and incubated at 37° C. and 5% CO₂ for 72 h. At the end of incubation time, the supernatant was collected and analyzed for secreted IL-17 using sandwich enzyme immunoassay (Mabtech AB, Sweden). The results were analyzed using Graphpad Prism and the half-maximal inhibitory concentrations (IC₅₀) of the test compounds were derived (Table 4).

TABLE 4
IC₅₀ values of selected compounds in luciferase and IL-17 assay.

| Example | Luc IC₅₀ (nM) | IL17 IC₅₀ (nM) |
|---|---|---|
| 1 | 2.1 | 6.6 |
| 2 | 7 | 25 |
| 3 | 8.5 | 38.3 |
| 4 | 1.46 | 15 |
| 5 | 5.6 | 17 |
| 6 | 3.4 | 35 |
| 7 | 8.7 | 10.4 |
| 8 | 11 | 2.7 |
| 9 | 8.9 | 9.2 |
| 10 | 6.8 | 9.4 |
| 11 | 15.9 | 14.7 |
| 12 | 5.9 | 10.6 |
| 13 | 8.9 | 27.2 |
| 14 | 1.3 | 9.5 |
| 15 | 5.7 | 27 |
| 16 | 0.9 | 42 |
| 17 | 1.9 | 6 |
| 18 | 3.9 | 23.7 |
| 19 | 1.58 | 27 |
| 20 | 15.75 | 11.3 |
| 23 | 1.4 | 21 |
| 25 | 1.5 | 14 |
| 26 | 2.5 | 7.3 |
| 27 | 12.98 | 10.8 |
| 28 | 5.8 | 41.6 |
| 29 | 19.41 | 19 |
| 31 | 12.6 | 50 |
| 32 | 5.96 | 18 |
| 33 | 20.18 | 24 |
| 35 | 2.3 | 12 |
| 36 | 2.44 | 19 |
| 38 | 1.5 | 23 |
| 39 | 22.54 | 22 |
| 41 | 0.76 | 15.5 |
| 44 | 10.3 | 43 |
| 46 | 12.86 | 17.1 |
| 47 | 10.1 | 17.9 |
| 48 | 10.5 | 38.9 |
| 49 | 4.7 | 17 |
| 50 | 14.5 | 30 |
| 51 | 12.12 | 16 |
| 53 | 1.9 | 48 |
| 58 | 3.4 | 23.3 |
| 61 | 9.2 | 35 |
| 63 | 11 | 18 |
| 64 | 15.9 | 63 |
| 65 | 2.4 | 6 |
| 68 | 9.6 | 42.7 |
| 69 | 6.4 | 22.4 |
| 70 | 17 | 22 |
| 71 | 15.7 | 32 |
| 72 | 20 | 20.8 |
| 73 | 17.6 | 23.4 |
| 74 | 23.9 | 23.8 |
| 78 | 7.9 | 47.3 |
| 79 | 12.3 | 39 |

The results above showed that compounds with present invention have shown much improved activity in both luciferase and IL-17 assay, when compared with compounds A and B shown in Table 1. Further confirmation of this activity was achieved by evaluating selected compounds for their in-vivo efficacy in animal models.

### In-Vivo Activity:

### Effect in Mouse Model of EAE:

EAE was induced in C57BL/6 wild-type mice by s.c. injection at four sites on the back with 200 μg/mouse MOG peptide in an emulsion with IFA supplemented with 5 mg/ml Mycobacterium tuberculosis, strain H37Ra. Pertussis toxin dissolved in PBS was injected i.p. at 200 ng/mouse at the time of immunization (Day 0) and 48 h later. Mice were scored daily on a scale of 0-5. 0, no clinical disease; 1, limp/flaccid tail; 2, moderate hind-limb weakness; 3, complete paralysis of hind-limbs; 4, complete hind-limb paralysis with partial forelimb paralysis; 5, death. All mice were 6-10 weeks of age when experiments were performed. Test compounds or its vehicle was administered per oral from day 0 to day 20.

Selected compounds have shown >90% inhibition of clinical score when given orally at 50 mg/kg BID.

### Effect in Collagen-Induced Arthritis Model

Male DBA1j (8 to 12-weeks old) mice were injected s.c with native bovine type II collagen (Chondrex, Redmond, Wash.), mixed with complete Freund's adjuvant at the ratio of 2:1, on days 0 and 21 at the base of the tail. Animals were observed for clinical score and assigned to different groups of similar score. Mice were then dosed and determined for clinical scores for 3 weeks. The degree of arthritis was determined based on a clinical score of 0-4 per paw and summed for all four paws.

Selected compound has shown 75% reduction in clinical score when given orally at 30 mg/kg BID.

### Effect in Imiquimod Induced Psoriasis Model

C57BL/6j Male mice (8-10 week-old at study initiation) were treated with imiquimod (IMQ) cream (5%) or petroleum (non-inflammatory inert cream). Mice were anesthetized before applying IMQ cream on to the skin. Test compounds or its vehicle was administered per oral one hour before the IMQ application. Treatment started at day 0 and continued twice a day for 6 days. The mice were scored daily for skin erythema and scaling. Ear thickness was measured daily using an engineer's caliper (Incyte) before the application of IMQ.

Selected compound has shown 40% reduction in ear weight when given orally at 3 mg/kg BID.

The pharmaceutical composition is provided by employing conventional techniques. Preferably the composition is in unit dosage form containing an effective amount of the active component, that is, the compounds of formula (I) according to this invention.

The quantity of active component, that is, the compounds of formula (I) according to this invention, in the pharmaceutical composition and unit dosage form thereof may be varied or adjusted widely depending upon the particular application method, the potency of the particular compound and the desired concentration. Generally, the quantity of active component will range between 0.5% to 90% by weight of the composition.

In one of the embodiments, the present invention of formula (I) can be co-administered in combination with one or more suitable pharmaceutically active agents. In a particular embodiment, the pharmaceutical compositions of the invention can be co-administered with or can include one or more other therapeutic compounds or adjuvants, such as but not limited to other (1) TNF-a Inhibitors; (2) non-selective COX-1/COX-2 inhibitors; (3) COX-2 inhibitors (4) other agents for inflammatory and autoimmune disease including glucocorticoids, methotrexate, leflunomide, sulfasalazine, azathioprine, cyclosporine, tacrolimus, penicillamine, bucillamine, actarit, mizoribine, lobenzarit, ciclesonide, hydroxychloroquin, d-penicillamine, aurothiomalate, auranofin or parenteral or oral gold, cyclophosphamide, Lymphostate-B, BAFF/APRIL inhibitors and CTLA-4-Ig or mimetic thereof, (5) leukotriene biosynthesis inhibitors, 5-lipoxygenase inhibitor or 5-lipoxygenase activating protein (FLAP) antagonist; (6) LTD4 receptor antagonist; (7) PDE4 inhibitors; (8) antihistamine HI receptor antagonists; (9) a1 and a2-adrenoceptor agonist; (10) anticholinergic agents; (11) β-adrenoceptor agonist (12) insulin-like growth factor type I (IGF-I) mimetic; (13) glucocorticosteroids; (14) kinase inhibitors such as inhibitors of Janus kinases (JAK 1 and/or JAK2 and/or JAK3 and/or TYK2), p38 MAPK and IKK2; (15) B-cell targeting biologies such as rituximab; (16) selective costimulation modulators such as abatacept; (17) interleukine inhibitors, such as IL-1 inhibitor anakinra, IL-6 inhibitor tocilizumab, and IL12/IL23 inhibitor ustekinumab. The compounds of the invention could also be combined with anti-IL17 antibodies to obtain additive/synergistic response for the treatment of inflammatory and autoimmune diseases.

1. Compound having the structure of general formula (I) 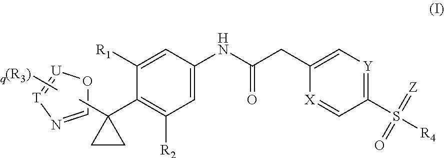
Wherein
Each of R₁ and R₂ are independently selected from halogen and (C₁-C₃)alkyl;
R₃ is selected from hydrogen, (C₁-C₆)alkyl, halo(C₁-C₆)alkyl, (C₆-C₁₀)aryl, (C₆-C₁₀)heteroaryl, (C₃-C₆)cycloalkyl, (C₄-C₆) heterocyclyl;
R₄ is selected from (C₁-C₃)alkyl, —NHR₆;
X and Y are each independently selected from CH or N atom;
Z is selected from NH or O atom;
T and U is independently selected from C or N atom with the proviso that both T and U cannot be simultaneously N atom
when R₃ is phenyl and both T and U represent carbon, then T and U can be fused with a phenyl ring to form radical of the following formula; 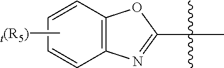
R₅ is selected from hydrogen, hydroxy, cyano, halogen, halo(C₁-C₆)alkyl, optionally substituted with (C₁-C₆)alkyl, —O(C₁-C₆alkyl), (C₃-C₆)cycloalkyl;
R₆ is (C₃-C₆)cycloalkyl;
when R₃ is substituted, the substituents on R₃ is selected from the group comprising of hydrogen, hydroxy, cyano, halogen, —OCF₃, halo(C₁-C₆)alkyl, optionally substituted (C₁-C₆)alkyl, —O(C₁-C₆alkyl), (C₃-C₆)cycloalkyl;
q is selected from integers from 1-2;
t is selected from integers from 1-4;

2. The compound as claimed in claim 1, wherein the ring formed by N, O, T, and U is selected from 1,2,4-Oxadiazole, 1,3,4-Oxadiazole, Oxazole and Benzoxazole.

3. The compound as claimed in claim 1, wherein the substituents on alkyl group is independently selected from hydroxy, —COOH, cyano, haloalkyl, aryl, heteroaryl, and cycloalkyl;

4. The compound as claimed in claim 1, wherein the alkyl group as used for R₃ is isopropyl, the aryl group as used for R₃ is phenyl, the heteroaryl group as used for R₃ is pyridine, the cycloalkyl group as used for R₃ is cyclopropyl, the heterocyclyl group as used for R₃ is tetrahydropyrane.

5. The compound as claimed in claim 1, wherein the compound is of formula (I-A):

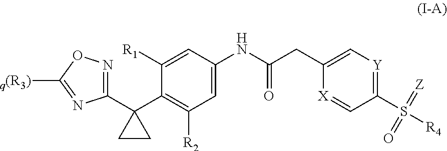

6. The compound as claimed in claim 1, wherein the compound is of formula (I-B):

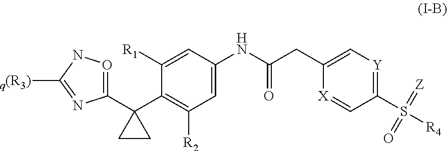

7. The compound as claimed in claim 1, wherein the compound is of formula (I-C):

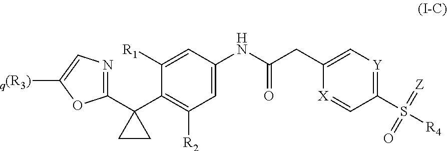

8. The compound as claimed in claim 1, wherein the compound is of formula (I-D):

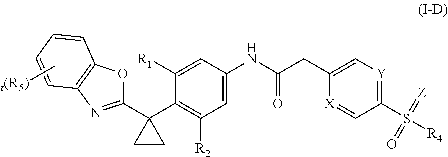

9. A compound as claimed in claim 1 selected from the group comprising of:
N-(3,5-dichloro-4-(1-(5-(4-fluorophenyl)-1,2,4-oxadiazol-3-yl)cyclopropyl)phenyl)-2-(4-(ethyl sulfonyl)phenyl)acetamide;
N-(3,5-dichloro-4-(1-(5-phenyl-1,2,4-oxadiazol-3-yl)cyclopropyl) phenyl)-2-(4-(ethyl sulfonyl)phenyl)acetamide;
N-(3,5-dichloro-4-(1-(5-(4-(trifluoromethyl)phenyl)-1,2,4-oxadiazol-3-yl)cyclopropyl) phenyl)-2-(4-(ethyl sulfonyl)phenyl)acetamide;
N-(3,5-dichloro-4-(1-(5-(p-tolyl)-1,2,4-oxadiazol-3-yl)cyclopropyl) phenyl)-2-(4-(ethyl sulfonyl)phenyl)acetamide;
N-(3,5-dichloro-4-(1-(5-(3,4-difluorophenyl)-1,2,4-oxadiazol-3-yl)cyclopropyl)phenyl)-2-(4-(ethyl sulfonyl)phenyl)acetamide;
N-(3,5-dichloro-4-(1-(5-(3-fluorophenyl)-1,2,4-oxadiazol-3-yl)cyclopropyl)phenyl)-2-(4-(ethyl sulfonyl)phenyl)acetamide;
N-(3,5-dichloro-4-(1-(5-(2,4-difluorophenyl)-1,2,4-oxadiazol-3-yl)cyclopropyl)phenyl)-2-(4-(ethyl sulfonyl)phenyl)acetamide;
N-(3,5-dichloro-4-(1-(5-(2,4,6-trifluorophenyl)-1,2,4-oxadiazol-3-yl)cyclopropyl) phenyl)-2-(4-(ethyl sulfonyl)phenyl)acetamide;
N-(3,5-dichloro-4-(1-(5-(2-chloro-4-fluorophenyl)-1,2,4-oxadiazol-3-yl)cyclopropyl) phenyl)-2-(4-(ethyl sulfonyl)phenyl)acetamide;
N-(3,5-dichloro-4-(1-(5-(3-chloro-4-fluorophenyl)-1,2,4-oxadiazol-3-yl)cyclopropyl) phenyl)-2-(4-(ethyl sulfonyl)phenyl)acetamide;
N-(3,5-dichloro-4-(1-(5-(3,4-dichlorophenyl)-1,2,4-oxadiazol-3-yl)cyclopropyl)phenyl)-2-(4-(ethyl sulfonyl)phenyl)acetamide;
N-(3,5-dichloro-4-(1-(5-(2,4-dichlorophenyl)-1,2,4-oxadiazol-3-yl)cyclopropyl)phenyl)-2-(4-(ethyl sulfonyl)phenyl)acetamide;
N-(3,5-dichloro-4-(1-(5-(4-chlorophenyl)-1,2,4-oxadiazol-3-yl)cyclopropyl)phenyl)-2-(4-(ethyl sulfonyl)phenyl)acetamide;
N-(3,5-dichloro-4-(1-(5-(4-cyanophenyl)-1,2,4-oxadiazol-3-yl)cyclopropyl)phenyl)-2-(4-(ethyl sulfonyl)phenyl)acetamide;
N-(3,5-dichloro-4-(1-(5-(2-fluoro-4-methoxyphenyl)-1,2,4-oxadiazol-3-yl) cyclopropyl) phenyl)-2-(4-(ethyl sulfonyl)phenyl)acetamide;
N-(3,5-dichloro-4-(1-(5-(thiophen-2-yl)-1,2,4-oxadiazol-3-yl)cyclopropyl)phenyl)-2-(4-(ethyl sulfonyl)phenyl)acetamide;
N-(3,5-dichloro-4-(1-(5-(5-chlorothiophen-2-yl)-1,2,4-oxadiazol-3-yl)cyclopropyl) phenyl)-2-(4-(ethyl sulfonyl)phenyl)acetamide;
N-(3,5-dichloro-4-(1-(5-(5-methylthiophen-2-yl)-1,2,4-oxadiazol-3-yl)cyclopropyl) phenyl)-2-(4-(ethyl sulfonyl)phenyl)acetamide;
N-(3,5-dichloro-4-(1-(5-(pyridin-3-yl)-1,2,4-oxadiazol-3-yl)cyclopropyl)phenyl)-2-(4-(ethyl sulfonyl)phenyl)acetamide;
N-(3,5-dichloro-4-(1-(5-(6-methoxypyridin-3-yl)-1,2,4-oxadiazol-3-yl)cyclopropyl) phenyl)-2-(4-(ethyl sulfonyl)phenyl)acetamide;
N-(3,5-dichloro-4-(1-(5-(4-fluorobenzyl)-1,2,4-oxadiazol-3-yl)cyclopropyl)phenyl)-2-(4-(ethyl sulfonyl)phenyl)acetamide;
N-(3,5-dichloro-4-(1-(5-(4-methoxyphenyl)-1,2,4-oxadiazol-3-yl)cyclopropyl)phenyl)-2-(4-(ethyl sulfonyl)phenyl)acetamide;
N-(3,5-dichloro-4-(1-(5-isopropyl-1,2,4-oxadiazol-3-yl)cyclopropyl)phenyl)-2-(4-(ethyl sulfonyl)phenyl)acetamide;
N-(4-(1-(1,2,4-oxadiazol-3-yl)cyclopropyl)-3,5-dichlorophenyl)-2-(4-(ethylsulfonyl) phenyl)acetamide;
N-(3,5-dichloro-4-(1-(5-cyclopropyl-1,2,4-oxadiazol-3-yl) cyclopropyl)phenyl)-2-(4-(ethyl sulfonyl)phenyl)acetamide;
N-(3,5-dichloro-4-(1-(5-(3,3-difluorocyclobutyl)-1,2,4-oxadiazol-3-yl)cyclopropyl) phenyl)-2-(4-(ethyl sulfonyl)phenyl)acetamide;
N-(3,5-dichloro-4-(1-(5-(tetrahydro-2H-pyran-4-yl)-1,2,4-oxadiazol-3-yl)cyclopropyl) phenyl)-2-(4-(ethyl sulfonyl)phenyl)acetamide;
N-(3,5-difluoro-4-(1-(5-(4-fluorophenyl)-1,2,4-oxadiazol-3-yl)cyclopropyl)phenyl)-2-(4-(ethyl sulfonyl)phenyl)acetamide;
N-(3-chloro-5-fluoro-4-(1-(5-(4-fluorophenyl)-1,2,4-oxadiazol-3-yl)cyclopropyl)phenyl)-2-(4-(ethyl sulfonyl)phenyl)acetamide;
N-(3-chloro-4-(1-(5-(4-chlorophenyl)-1,2,4-oxadiazol-3-yl)cyclopropyl)-5-fluorophenyl)-2-(4-(ethyl sulfonyl)phenyl)acetamide;
N-(3-chloro-5-fluoro-4-(1-(5-(4-(trifluoromethyl)phenyl)-1,2,4-oxadiazol-3-yl) cyclopropyl)phenyl)-2-(4-(ethyl sulfonyl)phenyl)acetamide;
N-(3-chloro-4-(1-(5-(4-cyanophenyl)-1,2,4-oxadiazol-3-yl)cyclopropyl)-5-fluorophenyl)-2-(4-(ethyl sulfonyl)phenyl)acetamide;
2-(4-(ethylsulfonyl)phenyl)-N-(4-(1-(5-(4-fluorophenyl)-1,2,4-oxadiazol-3-yl) cyclopropyl)-3,5-dimethylphenyl)acetamide;
N-(3,5-dichloro-4-(1-(5-(4-(trifluoromethoxy)phenyl)-1,2,4-oxadiazol-3-yl) cyclopropyl) phenyl)-2-(4-(ethyl sulfonyl)phenyl)acetamide;
N-(3,5-dichloro-4-(1-(3-(4-fluorophenyl)-1,2,4-oxadiazol-5-yl)cyclopropyl)phenyl)-2-(4-(ethyl sulfonyl)phenyl)acetamide;
N-(3,5-dichloro-4-(1-(6-fluorobenzo[d]oxazol-2-yl)cyclopropyl) phenyl)-2-(4-(ethyl sulfonyl)phenyl)acetamide;
2-(4-(ethylsulfonyl)phenyl)-N-(4-(1-(6-fluorobenzo[d]oxazol-2-yl)cyclopropyl)-3,5-dimethylphenyl)acetamide;
N-(3,5-dichloro-4-(1-(5-(4-fluorophenyl)oxazol-2-yl)cyclopropyl)phenyl)-2-(4-(ethyl sulfonyl)phenyl)acetamide;
N-(3,5-dichloro-4-(1-(5-(4-chlorophenyl)oxazol-2-yl)cyclopropyl) phenyl)-2-(4-(ethyl sulfonyl)phenyl)acetamide;
N-(3,5-dichloro-4-(1-(5-(3-fluoro-4-methoxyphenyl)oxazol-2-yl)cyclopropyl)phenyl)-2-(4-(ethyl sulfonyl)phenyl)acetamide;
N-(3,5-dichloro-4-(1-(5-(4-fluorophenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(4-(trifluoromethyl)phenyl)-1,2,4-oxadiazol-3-yl)cyclopropyl) phenyl)-2-(5-(ethyl sulfonyl)pyridin-2-yl)acetamide;
N-(3,5-dichloro-4-(1-(5-(p-tolyl)-1,2,4-oxadiazol-3-yl)cyclopropyl) phenyl)-2-(5-(ethylsulfonyl)pyridin-2-yl)acetamide;
N-(3,5-dichloro-4-(1-(5-(3,4-difluorophenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(3,4,5-trifluorophenyl)-1,2,4-oxadiazol-3-yl)cyclopropyl) phenyl)-2-(5-(ethylsulfonyl)pyridin-2-yl)acetamide;
N-(3,5-dichloro-4-(1-(5-(2-chloro-4-fluorophenyl)-1,2,4-oxadiazol-3-yl)cyclopropyl) phenyl)-2-(5-(ethyl sulfonyl)pyridin-2-yl)acetamide;
N-(3,5-dichloro-4-(1-(5-(3-chloro-4-fluorophenyl)-1,2,4-oxadiazol-3-yl)cyclopropyl) phenyl)-2-(5-(ethyl sulfonyl)pyridin-2-yl)acetamide;
N-(3,5-dichloro-4-(1-(5-(3,4-dichlorophenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(2,4-dichlorophenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(4-chlorophenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(3-chlorophenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(4-methoxyphenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(4-cyanophenyl)-1,2,4-oxadiazol-3-yl)cyclopropyl)phenyl)-2-(5-(ethylsulfonyl)pyridin-2-yl)acetamide;
N-(3,5-dichloro-4-(1-(5-(5-chlorothiophen-2-yl)-1,2,4-oxadiazol-3-yl)cyclopropyl) phenyl)-2-(5-(ethylsulfonyl)pyridin-2-yl)acetamide;
N-(3,5-dichloro-4-(1-(5-(3,3-difluorocyclobutyl)-1,2,4-oxadiazol-3-yl)cyclopropyl) phenyl)-2-(5-(ethyl sulfonyl)pyridin-2-yl)acetamide;
N-(3,5-dichloro-4-(1-(5-cyclopropyl-1,2,4-oxadiazol-3-yl)cyclopropyl)phenyl)-2-(5-(ethylsulfonyl)pyridin-2-yl)acetamide;
2-(5-(ethylsulfonyl)pyridin-2-yl)-N-(4-(1-(5-(4-fluorophenyl)-1,2,4-oxadiazol-3-yl)cyclopropyl)-3,5-dimethylphenyl)acetamide;
N-(3,5-dichloro-4-(1-(6-fluorobenzo[d]oxazol-2-yl)cyclopropyl) phenyl)-2-(5-(ethylsulfonyl)pyridin-2-yl)acetamide;
2-(5-(ethylsulfonyl)pyridin-2-yl)-N-(4-(1-(6-fluorobenzo[d]oxazol-2-yl)cyclopropyl)-3,5-dimethylphenyl)acetamide;
N-(3,5-dichloro-4-(1-(5-(4-fluorophenyl)oxazol-2-yl)cyclopropyl)phenyl)-2-(5-(ethylsulfonyl)pyridin-2-yl)acetamide
N-(3,5-dichloro-4-(1-(5-(4-fluorophenyl)-1,2,4-oxadiazol-3-yl)cyclopropyl)phenyl)-2-(4-(ethyl sulfonimidoyl)phenyl)acetamide;
N-(3,5-dichloro-4-(1-(5-(4-(trifluoromethyl)phenyl)-1,2,4-oxadiazol-3-yl)cyclopropyl) phenyl)-2-(4-(ethyl sulfonimidoyl)phenyl)acetamide;
2-(4-(N-cyclopropylsulfamoyl)phenyl)-N-(3,5-dichloro-4-(1-(5-(4-fluorophenyl)-1,2,4-oxadiazol-3-yl)cyclopropyl)phenyl)acetamide;
2-(4-(N-cyclopropylsulfamoyl)phenyl)-N-(3,5-dichloro-4-(1-(5-(4-(trifluoromethyl) phenyl)-1,2,4-oxadiazol-3-yl)cyclopropyl)phenyl)acetamide;
2-(4-(N-cyclopropylsulfamoyl)phenyl)-N-(3,5-dichloro-4-(1-(6-fluorobenzo[d]oxazol-2-yl)cyclopropyl)phenyl)acetamide;
N-(3,5-dichloro-4-(1-(5-(4-fluorophenyl)-1,2,4-oxadiazol-3-yl)cyclopropyl)phenyl)-2-(6-(ethylsulfonyl)pyridin-3-yl)acetamide;
N-(3,5-dichloro-4-(1-(5-(4-(trifluoromethyl)phenyl)-1,2,4-oxadiazol-3-yl)cyclopropyl) phenyl)-2-(6-(ethylsulfonyl)pyridin-3-yl)acetamide;
N-(3,5-dichloro-4-(1-(5-(3,4-difluorophenyl)-1,2,4-oxadiazol-3-yl)cyclopropyl)phenyl)-2-(6-(ethylsulfonyl)pyridin-3-yl)acetamide;
N-(3,5-dichloro-4-(1-(5-(2,4-difluorophenyl)-1,2,4-oxadiazol-3-yl)cyclopropyl)phenyl)-2-(6-(ethylsulfonyl)pyridin-3-yl)acetamide;
N-(3,5-dichloro-4-(1-(5-(3-chloro-4-fluorophenyl)-1,2,4-oxadiazol-3-yl)cyclopropyl) phenyl)-2-(6-(ethylsulfonyl)pyridin-3-yl)acetamide;
N-(3,5-dichloro-4-(1-(5-(3,4-dichlorophenyl)-1,2,4-oxadiazol-3-yl)cyclopropyl)phenyl)-2-(6-(ethylsulfonyl)pyridin-3-yl)acetamide;
N-(3,5-dichloro-4-(1-(5-(4-chlorophenyl)-1,2,4-oxadiazol-3-yl)cyclopropyl)phenyl)-2-(6-(ethylsulfonyl)pyridin-3-yl)acetamide;
N-(3,5-dichloro-4-(1-(5-(4-cyanophenyl)-1,2,4-oxadiazol-3-yl)cyclopropyl)phenyl)-2-(6-(ethylsulfonyl)pyridin-3-yl)acetamide;
N-(3,5-dichloro-4-(1-(6-fluorobenzo[d]oxazol-2-yl)cyclopropyl)phenyl)-2-(6-(ethylsulfonyl)pyridin-3-yl)acetamide;
2-(6-(ethylsulfonyl)pyridin-3-yl)-N-(4-(1-(5-(4-fluorophenyl)-1,2,4-oxadiazol-3-yl)cyclopropyl)-3,5-dimethylphenyl)acetamide;
2-(6-(ethylsulfonyl)pyridin-3-yl)-N-(4-(1-(6-fluorobenzo[d]oxazol-2-yl)cyclopropyl)-3,5-dimethylphenyl)acetamide;
N-(3,5-dichloro-4-(1-(5-(4-fluorophenyl)-1,2,4-oxadiazol-3-yl)cyclopropyl)phenyl)-2-(5-(ethylsulfonyl)pyrazin-2-yl)acetamide;
N-(3,5-dichloro-4-(1-(6-fluorobenzo[d]oxazol-2-yl)cyclopropyl) phenyl)-2-(5-(ethylsulfonyl)pyrazin-2-yl)acetamide;
N-(3,5-dichloro-4-(1-(5-(4-fluorophenyl)-1,3,4-oxadiazol-2-yl)cyclopropyl)phenyl)-2-(4-(ethyl sulfonyl)phenyl)acetamide;
N-(3,5-dichloro-4-(1-(5-(4-fluorophenyl)-1,3,4-oxadiazol-2-yl)cyclopropyl)phenyl)-2-(5-(ethylsulfonyl)pyridin-2-yl)acetamide;
N-(3,5-dichloro-4-(1-(5-(4-fluorophenyl)-1,2,4-oxadiazol-3-yl)cyclopropyl)phenyl)-2-(4-(methyl sulfonyl)phenyl)acetamide;
N-(3,5-dichloro-4-(1-(5-(4-chlorophenyl)-1,2,4-oxadiazol-3-yl)cyclopropyl)phenyl)-2-(4-(methylsulfonyl)phenyl)acetamide;
N-(3,5-dichloro-4-(1-(5-(4-(trifluoromethyl)phenyl)-1,2,4-oxadiazol-3-yl)cyclopropyl) phenyl)-2-(4-(methylsulfonyl)phenyl)acetamide;
N-(3,5-dichloro-4-(1-(5-(4-cyanophenyl)-1,2,4-oxadiazol-3-yl) cyclopropyl)phenyl)-2-(4-(methylsulfonyl)phenyl)acetamide;
N-(3,5-dichloro-4-(1-(5-(3-chloro-4-fluorophenyl)-1,2,4-oxadiazol-3-yl)cyclopropyl) phenyl)-2-(4-(methyl sulfonyl)phenyl)acetamide;
N-(3,5-dichloro-4-(1-(6-fluorobenzo[d]oxazol-2-yl) cyclopropyl) phenyl)-2-(4-(methylsulfonyl)phenyl)acetamide;
N-(3,5-dichloro-4-(1-(3-(4-chlorophenyl)-1,2,4-oxadiazol-5-yl) cyclopropyl)phenyl)-2-(4-(ethyl sulfonyl)phenyl)acetamide;
N-(3,5-dichloro-4-(1-(3-(4-(trifluoromethyl)phenyl)-1,2,4-oxadiazol-5-yl)cyclopropyl) phenyl)-2-(4-(ethyl sulfonyl)phenyl)acetamide;
N-(3,5-dichloro-4-(1-(3-(4-cyanophenyl)-1,2,4-oxadiazol-5-yl) cyclopropyl)phenyl)-2-(4-(ethyl sulfonyl)phenyl)acetamide;
N-(3,5-dichloro-4-(1-(3-(3,4-difluorophenyl)-1,2,4-oxadiazol-5-yl) cyclopropyl)phenyl)-2-(4-(ethyl sulfonyl)phenyl)acetamide;
N-(3,5-dichloro-4-(1-(3-(3-chloro-4-fluorophenyl)-1,2,4-oxadiazol-5-yl)cyclopropyl) phenyl)-2-(4-(ethyl sulfonyl)phenyl)acetamide;
N-(3,5-dichloro-4-(1-(3-(2,4-difluorophenyl)-1,2,4-oxadiazol-5-yl) cyclopropyl)phenyl)-2-(4-(ethyl sulfonyl)phenyl)acetamide;
N-(3,5-dichloro-4-(1-(3-(p-tolyl)-1,2,4-oxadiazol-5-yl) cyclopropyl) phenyl)-2-(4-(ethyl sulfonyl)phenyl)acetamide;
N-(3,5-dichloro-4-(1-(3-phenyl-1,2,4-oxadiazol-5-yl)cyclopropyl) phenyl)-2-(4-(ethyl sulfonyl)phenyl)acetamide;
N-(3,5-dichloro-4-(1-(3-cyclopropyl-1,2,4-oxadiazol-5-yl) cyclopropyl)phenyl)-2-(4-(ethyl sulfonyl)phenyl)acetamide;
N-(3,5-dichloro-4-(1-(3-(3,3-difluorocyclobutyl)-1,2,4-oxadiazol-5-yl) cyclopropyl) phenyl)-2-(4-(ethyl sulfonyl)phenyl)acetamide;
N-(3,5-dichloro-4-(1-(3-(2-fluoro-4-methoxyphenyl)-1,2,4-oxadiazol-5-yl) cyclopropyl) phenyl)-2-(4-(ethyl sulfonyl)phenyl)acetamide;
2-(4-(ethylsulfonyl)phenyl)-N-(4-(1-(3-(4-fluorophenyl)-1,2,4-oxadiazol-5-yl) cyclopropyl)-3,5-dimethylphenyl)acetamide;
2-(4-(ethylsulfonyl)phenyl)-N-(4-(1-(5-(4-fluorophenyl)oxazol-2-yl)cyclopropyl)-3,5-dimethylphenyl)acetamide;
N-(3,5-dichloro-4-(1-(5-(4-chlorophenyl)-1,2,4-oxadiazol-3-yl) cyclopropyl)phenyl)-2-(4-(ethyl sulfonimidoyl)phenyl)acetamide;
N-(3,5-dichloro-4-(1-(6-fluorobenzo[d]oxazol-2-yl) cyclopropyl) phenyl)-2-(4-(ethylsulfonimidoyl)phenyl)acetamide;
N-(3,5-dichloro-4-(1-(6-chlorobenzo[d]oxazol-2-yl) cyclopropyl) phenyl)-2-(4-(ethylsulfonimidoyl)phenyl)acetamide;
N-(4-(1-(benzo[d]oxazol-2-yl)cyclopropyl)-3,5-dichlorophenyl)-2-(4-(ethylsulfonyl) phenyl)acetamide;
N-(3,5-dichloro-4-(1-(6-chlorobenzo[d]oxazol-2-yl)cyclopropyl) phenyl)-2-(4-(ethyl sulfonyl)phenyl)acetamide;
N-(3,5-dichloro-4-(1-(6-(trifluoromethyl)benzo[d]oxazol-2-yl)cyclopropyl)phenyl)-2-(4-(ethyl sulfonyl)phenyl)acetamide;
N-(4-(1-(benzo[d]oxazol-2-yl)cyclopropyl)-3,5-dichlorophenyl)-2-(5-(ethylsulfonyl) pyridin-2-yl)acetamide;
N-(3,5-dichloro-4-(1-(6-chlorobenzo[d]oxazol-2-yl)cyclopropyl) phenyl)-2-(5-(ethylsulfonyl)pyridin-2-yl)acetamide;
N-(3,5-dichloro-4-(1-(6-(trifluoromethyl)benzo[d]oxazol-2-yl)cyclopropyl)phenyl)-2-(5-(ethylsulfonyl)pyridin-2-yl)acetamide;
N-(4-(1-(6-chlorobenzo[d]oxazol-2-yl)cyclopropyl)-3,5-dimethylphenyl)-2-(5-(ethylsulfonyl)pyridin-2-yl)acetamide;
N-(3,5-dichloro-4-(1-(5-(4-chlorophenyl)-1,3,4-oxadiazol-2-yl)cyclopropyl)phenyl)-2-(4-(ethyl sulfonyl)phenyl)acetamide;
N-(3,5-dichloro-4-(1-(5-(4-(trifluoromethyl)phenyl)-1,3,4-oxadiazol-2-yl)cyclopropyl)phenyl)-2-(4-(ethyl sulfonyl)phenyl)acetamide;
N-(4-(1-(5-(4-chlorophenyl)-1,3,4-oxadiazol-2-yl)cyclopropyl)-3,5-dimethylphenyl)-2-(4-(ethyl sulfonyl)phenyl)acetamide;
2-(4-(N-cyclopropylsulfamoyl)phenyl)-N-(3,5-dichloro-4-(1-(5-(4-chlorophenyl)-1,2,4-oxadiazol-3-yl)cyclopropyl)phenyl)acetamide;
2-(4-(N-cyclopropylsulfamoyl)phenyl)-N-(3,5-dichloro-4-(1-(6-chlorobenzo[d]oxazol-2-yl)cyclopropyl)phenyl)acetamide;
N-(3,5-dichloro-4-(1-(5-(4-cyclopropylphenyl)-1,2,4-oxadiazol-3-yl)cyclopropyl) phenyl)-2-(4-(ethyl sulfonyl)phenyl)acetamide;
N-(4-(1-(5-(4-chlorophenyl)-1,2,4-oxadiazol-3-yl)cyclopropyl)-3,5-difluorophenyl)-2-(4-(ethyl sulfonyl)phenyl)acetamide;
N-(3,5-difluoro-4-(1-(5-(4-(trifluoromethyl)phenyl)-1,2,4-oxadiazol-3-yl)cyclopropyl) phenyl)-2-(4-(ethyl sulfonyl)phenyl)acetamide;
N-(4-(1-(5-(4-cyanophenyl)-1,2,4-oxadiazol-3-yl)cyclopropyl)-3,5-difluorophenyl)-2-(4-(ethyl sulfonyl)phenyl)acetamide;
N-(4-(1-(5-(4-chlorophenyl)-1,2,4-oxadiazol-3-yl)cyclopropyl)-3,5-dimethylphenyl)-2-(4-(ethyl sulfonyl)phenyl)acetamide;
N-(3,5-dimethyl-4-(1-(5-(4-(trifluoromethyl)phenyl)-1,2,4-oxadiazol-3-yl)cyclopropyl) phenyl)-2-(4-(ethyl sulfonyl)phenyl)acetamide;
N-(4-(1-(5-(4-cyanophenyl)-1,2,4-oxadiazol-3-yl)cyclopropyl)-3,5-dimethylphenyl)-2-(4-(ethyl sulfonyl)phenyl)acetamide.

10. A pharmaceutical composition comprising a therapeutically effective amount of a compound of Formula (I) as claimed in claim 1 and optionally one or more pharmaceutically acceptable carriers, diluents or excipients.

11. A method of treating diseases medicated by the RORγ which comprising administering to a patient in need thereof an effective amount of a compound of Formula (I) as claimed in claim 1 or its suitable pharmaceutical composition.

12. The use of compounds of formula (I) or its pharmaceutical compositions as claimed in claim 1 suitable for treatment of diseases wherein the RORγ has a pathophysiological function.

13. The pharmaceutical composition as claimed in claim 7 in combination with suitable (1) TNF-a Inhibitors; (2) non-selective COX-1/COX-2 inhibitors; (3) COX-2 inhibitors (4) and other agents suitable for inflammatory and autoimmune disease including glucocorticoids, methotrexate, leflunomide, sulfasalazine, azathioprine, cyclosporine, tacrolimus, penicillamine, bucillamine, actarit, mizoribine, lobenzarit, ciclesonide, hydroxychloroquin, d-penicillamine, aurothiomalate, auranofin or parenteral or oral gold, cyclophosphamide, lymphostate-B, BAFF/APRIL inhibitors and CTLA-4-Ig or mimetic thereof, (5) leukotriene biosynthesis inhibitors, 5-lipoxygenase inhibitor or 5-lipoxygenase activating protein (FLAP) antagonist; (6) LTD4 receptor antagonist; (7) PDE4 inhibitors; (8) antihistamine HI receptor antagonists; (9) a1 and a2-adrenoceptor agonist; (10) anticholinergic agents; (11) β-adrenoreceptor agonist (12) insulin-like growth factor type I (IGF-I) mimetic; (13) glucocorticosteroids; (14) kinase inhibitors selected from inhibitors of Janus kinases (JAK 1 and/or JAK2 and/or JAK3 and/or TYK2), p38 MAPK and IKK2; (15) B-cell targeting biologies such as rituximab; (16) selective costimulation modulators such as abatacept; (17) interleukine inhibitors, selected from IL-1 inhibitor anakinra, IL-6 inhibitor tocilizumab, and IL12/IL23 inhibitor ustekinumab.
